(19) United States
(12) Patent Application Publication
(10) Pub. No.: US 2025/0275993 A1
(43) Pub. Date: Sep. 4, 2025

(54) GLYCOSYLATION INHIBITORS AS THERAPEUTICS FOR STROKE

(71) Applicant: The Regents of the University of California, Oakland, CA (US)

(72) Inventors: Napoleone Ferrara, San Diego, CA (US); Pin Li, San Diego, CA (US)

(21) Appl. No.: 18/920,285

(22) Filed: Oct. 18, 2024

Related U.S. Application Data
(63) Continuation of application No. PCT/US2023/018909, filed on Apr. 18, 2023.
(60) Provisional application No. 63/332,932, filed on Apr. 20, 2022.

(51) Int. Cl. A61K 31/7008; A61K 31/437; A61K 38/18; A61P 9/10; A61P 25/00

(52) U.S. Cl. CPC A61K 31/7008; A61K 31/437; A61K 38/1866; A61P 9/10; A61P 25/00

(57) ABSTRACT

Pharmaceutical compositions for treating an ischemic condition in a subject may include inhibitors of protein glycosylation. Methods may include administering to the subject in need thereof an effective amount an inhibitor of protein glycosylation. The administration may be effective to promote endothelial cell proliferation and angiogenesis in the subject. The subject may be in need of induced angiogenesis due to an ischemic condition caused by a stroke. Pharmaceutical compositions for inhibiting protein glycosylation in a cell may include ManN. Methods for inhibiting protein glycosylation in a cell may include administering to a cell an effective amount of ManN.

### CROSS-REFERENCE

This application is a continuation of PCT/US2023/018909, filed Apr. 18, 2023, which claims priority to U.S. Provisional Application No. 63/332,932, filed Apr. 20, 2022, the entire contents of which are incorporated by reference herewith.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Angiogenesis is a complex process involving the growth of new blood vessels from the existing vasculature and occurs in both physiological and pathological circumstances. In tumors, angiogenesis facilitates rapid growth and metastasis through delivery of nutrients and oxygen and removal of metabolic wastes. Development of the vasculature requires the coordinated activation of multiple signaling pathways, including VEGF/VEGFR, angiopoietin (Ang)/Tie2, Notch, Ephrin/Eph and PDGF/PDGFR.

Stimulating angiogenesis has the potential of facilitating treatment of a number of conditions characterized by reduced perfusion, including diabetic ulcers, myocardial and limb ischemia. Conversely, blocking angiogenesis is a clinically validated strategy to treat malignant tumors and intraocular neovascular disorders.

Endothelial cell (EC) metabolism is hypothesized to play a key role in the regulation of angiogenesis in normal and pathological circumstances. Metabolic switches in ECs, such as fatty acid, glucose, and glutamine metabolism, have been reported to trigger angiogenesis. ECs in the tumor vasculature are known to rely on glycolysis for ATP production, for instance through enhanced expression of glucose transporter GLUT1.

Lowering glycolysis in tumor ECs arrests their proliferation. In addition, aberrant glycosylation patterns have been documented during oncogenic transformation and progression of cancer and it has been proposed that inhibiting glycosylation may result in suppression of key angiogenesis pathways, including VEGF/VEGFR2 and Notch. Evidence that has emerged in recent years points to glycans as novel angiogenesis regulators due to changes in protein glycosylation. For example, the glycan-binding protein Galectin1 has been reported to interact with VEGFR2, leading to ligand-independent receptor activation, which may contribute to tumor resistance to anti-VEGF therapy. Therefore, EC metabolism has been identified as a new target for anti-angiogenic therapy, particularly through inhibition of energy metabolism and glycosylation.

### SUMMARY

There remains a need for effective therapeutics and methods of treatment for stroke. Stroke continues to be a debilitating condition that can lead to extensive rehabilitating periods, to decreases in an ability to conduct activities of daily living, to social and financial hardships, and can be fatal. Preventing a first stroke, preventing a recurrence of a stroke, mitigating the effects of an ongoing stroke, or improving the treatment and recovery from a stroke would all present great benefits to society and improve quality of life for many individuals.

The present disclosure provides pharmaceutical compositions and methods for treating an ischemic condition in a subject caused by a stroke. In some embodiments, a pharmaceutical composition for treating a stroke comprises an effective amount of an N-glycosylation inhibitor administered to a subject in need thereof. In some embodiments, the N-glycosylation inhibitor comprises hexosamine D-mannosamine (ManN), Kifunensine (Kif), Castanospermine (Cas), or a combination thereof. In some embodiments, the N-glycosylation inhibitor comprises ManN. In some embodiments, the subject has experienced an ischemic stroke. In some embodiments, the ischemic stroke comprises a thrombotic stroke or an embolic stroke. In some embodiments, the subject has experienced a hemorrhagic stroke. In some embodiments, the hemorrhagic stroke comprises a subarachnoid hemorrhage or an intracerebral hemorrhage. In some embodiments, the subject has experienced a brainstem stroke. In some embodiments, the subject has experienced a transient ischemic attack. In some embodiments, the subject has experienced a cryptogenic stroke.

Described herein are pharmaceutical compositions providing an effective amount of an N-glycosylation inhibitor to treat stroke. In some embodiments, the effective amount of an N-glycosylation inhibitor comprises a single dosing treatment regimen. In some embodiments, the single dosing treatment regimen comprises pretreatment, concurrent treatment, or post-treatment in relation to an ischemic event affecting the brain. In some embodiments, the effective amount of an N-glycosylation inhibitor comprises a multiple dosing treatment regimen. In some embodiments, the multiple dosing treatment regimen comprises pretreatment, concurrent treatment, and/or post-treatment in relation to an ischemic event affecting the brain. In some embodiments, the effective amount of an N-glycosylation inhibitor is administered orally, intravenously, intrathecally or intraperitoneally. In some embodiments, the effective amount of an N-glycosylation inhibitor comprises about 10 mg to about 100 g of the N-glycosylation inhibitor. In some embodiments, the effective amount of an N-glycosylation inhibitor comprises about 200 mg to about 40 g of the N-glycosylation inhibitor. In some embodiments, the effective amount of an N-glycosylation inhibitor comprises about 5 g to about 20 g of the N-glycosylation inhibitor.

Described herein are pharmaceutical compositions providing an effective amount of an N-glycosylation inhibitor to treat stroke that may be combined with other factors that modulate angiogenesis. In some embodiments, pharmaceutical compositions described herein further comprise an effective amount of a pro-angiogenic factor. In some embodiments, the pro-angiogenic factor comprises vascular endothelial growth factor (VEGF), or a derivative thereof. In some embodiments, the VEGF is VEGF-A, VEGF-B, VEGF-C, VEGF-D, placental growth factor (PLGF), or a combination thereof. In some embodiments, the VEGF is a recombinant VEGF. In some embodiments, the VEGF is administered locally near a site in the brain of a subject affected by an ischemic event.

Described herein are methods for treating stroke comprising administering to a subject in need thereof an effective amount of an N-glycosylation inhibitor. In some embodiments, the N-glycosylation inhibitor comprises hexosamine D-mannosamine (ManN), Kifunensine (Kif), Castanospermine (Cas), or a combination thereof. In some embodiments, the N-glycosylation inhibitor comprises ManN. In some embodiments, the subject has experienced an ischemic stroke. In some embodiments, the ischemic stroke is a thrombotic stroke or an embolic stroke. In some embodiments, the subject has experienced a hemorrhagic stroke. In some embodiments, the hemorrhagic stroke comprises a subarachnoid hemorrhage or an intracerebral hemorrhage. In some embodiments, the subject has experienced a brainstem stroke. In some embodiments, the subject has experienced a transient ischemic attack. In some embodiments, the subject has experienced a cryptogenic stroke.

Described herein are methods for treating stroke comprising administering to a subject in need thereof an effective amount of an N-glycosylation inhibitor. In some embodiments, the effective amount of an N-glycosylation inhibitor comprises a single dosing treatment regimen. In some embodiments, the single dosing treatment regimen comprises pretreatment, concurrent treatment, or post-treatment in relation to an ischemic event affecting the brain. In some embodiments, the effective amount of an N-glycosylation inhibitor comprises a multiple dosing treatment regimen. In some embodiments, the multiple dosing treatment regimen comprises pretreatment, concurrent treatment, and/or post-treatment in relation to an ischemic event affecting the brain. In some embodiments, the effective amount of an N-glycosylation inhibitor is administered orally, intravenously, intrathecally or intraperitoneally. In some embodiments, the effective amount of an N-glycosylation inhibitor comprises about 10 mg to about 300 g of the N-glycosylation inhibitor. In some embodiments, the effective amount of an N-glycosylation inhibitor comprises about 200 mg to about 100 g of the N-glycosylation inhibitor. In some embodiments, the effective amount of an N-glycosylation inhibitor comprises about 5 g to about 40 g of the N-glycosylation inhibitor. In some embodiments, the single dosing treatment regimen comprises administering a formulation of the N-glycosylation inhibitor at a dosage of about 20 mg to about 2000 mg/kg body weight of a subject. In some embodiments, the single dosing treatment regimen comprises administering a formulation of the N-glycosylation inhibitor at a dosage of about 100 mg to about 300 mg/kg body weight of a subject. In some embodiments, the multiple dosing treatment regimen comprises administering a formulation of the N-glycosylation inhibitor at a dosage of about 10 mg to about 2000 mg/kg body weight of a subject. In some embodiments, the multiple dosing treatment regimen comprises administering a formulation of the N-glycosylation inhibitor at a dosage of about 100 mg to about 300 mg/kg body weight of a subject.

Described herein are methods of treating stroke comprising administering to a subject in need thereof an effective amount of an N-glycosylation inhibitor that may be combined with other factors that modulate angiogenesis. In some embodiments, the administering to a subject further comprises administering an effective amount of a pro-angiogenic factor. In some embodiments, the pro-angiogenic factor comprises vascular endothelial growth factor (VEGF), or a derivative thereof. In some embodiments, the VEGF is VEGF-A, VEGF-B, VEGF-C, VEGF-D, placental growth factor (PLGF), or a combination thereof. In some embodiments, the VEGF is a recombinant VEGF. In some embodiments, the VEGF is administered locally near a site in the brain of a subject affected by an ischemic event.

Described herein are methods of treating stroke comprising administering to a subject in need thereof an effective amount of an N-glycosylation inhibitor wherein N-linked glycosylation of proteins is inhibited in an endothelial cell in a subject. Also described herein are methods of treating stroke comprising administering to a subject in need thereof an effective amount of an N-glycosylation inhibitor wherein the administering is effective to stimulate endothelial cell proliferation and angiogenesis of blood vessels near the brain. Also described herein are methods of treating stroke comprising administering to a subject in need thereof an effective amount of an N-glycosylation inhibitor wherein the administering is effective to stimulate brain endothelial cell proliferation and angiogenesis. Also described herein are methods of treating stroke comprising administering to a subject in need thereof an effective amount of an N-glycosylation inhibitor wherein the administering is effective to activate JNK signaling and upregulate an unfolded protein response caused by endoplasmic reticulum stress. Also described herein are methods of treating stroke comprising administering to a subject in need thereof an effective amount of an N-glycosylation inhibitor wherein the subject demonstrates an improvement in one or more symptoms of stroke following the administering. In some embodiments, the one or more symptoms are selected from the group consisting of: muscle weakness, numbness in the face, paralysis in the face, paralysis in a limb, paralysis on one side of the body, slurred speech, garbled speech, difficulty understanding others, sudden onset blindness in one or both eyes, double vision, vertigo, confusion, lack of mental alertness, loss of balance, loss of coordination, stroke-induced mortality, hypoxic damage to one or more areas of the brain, stroke-induced memory impairment, stroke-induced voluntary movement impairment, stroke-induced language impairment, stroke-induced reduction in cognitive capacity, stroke-induced reduction in mobility. In some embodiments of methods of treating stroke described herein, the subject demonstrates a significant increase in vascular density in or near a region of stroke-induced brain injury following the administering.

In some embodiments, a pharmaceutical composition for treating an ischemic condition in a subject caused by a stroke includes inhibitors of N-glycosylation. In some embodiments, a pharmaceutical composition for treating an ischemic condition in a subject caused by a stroke includes hexosamine D-mannosamine (ManN). In some embodiments, a method for treating an ischemic condition in a subject caused by a stroke includes administering to the subject in need thereof an effective amount of hexosamine D-mannosamine (ManN). The present disclosure also provides pharmaceutical compositions and methods for preventing the occurrence of a stroke in a subject. The present disclosure also provides pharmaceutical compositions and methods for attenuating the effects of an ongoing ischemic event affecting the brain.

In some embodiments, the administration is effective to promote endothelial cell proliferation and angiogenesis in the subject. In some embodiments, the method further includes administering to the subject in need thereof an effective amount of an N-glycosylation inhibitor. In some embodiments, the method further includes administering to the subject in need thereof an effective amount of VEGF.

Described herein are methods for treating stroke comprising administering to a subject in need thereof an effective amount of an N-glycosylation inhibitor. In some embodiments, N-linked glycosylation of proteins is inhibited in an endothelial cell in a subject. In some embodiments, the administration is effective to stimulate endothelial cell proliferation and angiogenesis of blood vessels near the brain. In some embodiments, the administration is effective to stimulate brain endothelial cell proliferation and angiogenesis. In some embodiments, the administration is effective to activate JNK signaling and upregulate an unfolded protein response caused by endoplasmic reticulum stress.

Described herein are methods of preventing stroke comprising administering to a subject in need thereof an effective amount of an N-glycosylation inhibitor. In some embodiments, the N-glycosylation inhibitor comprises hexosamine D-mannosamine (ManN), Kifunensine (Kif), Castanospermine (Cas), or a combination thereof. In some embodiments, the N-glycosylation inhibitor comprises ManN. In some embodiments, the effective amount of an N-glycosylation inhibitor comprises a single dosing treatment regimen. In some embodiments, the effective amount of an N-glycosylation inhibitor comprises a multiple dosing treatment regimen. In some embodiments, the effective amount of an N-glycosylation inhibitor is administered orally, intravenously, intrathecally or intraperitoneally. In some embodiments, the effective amount of an N-glycosylation inhibitor comprises about 10 mg to about 300 g of the N-glycosylation inhibitor. In some embodiments, the effective amount of an N-glycosylation inhibitor comprises about 200 mg to about 100 g of the N-glycosylation inhibitor. In some embodiments, the effective amount of an N-glycosylation inhibitor comprises about 5 g to about 40 g of the N-glycosylation inhibitor.

Described herein are methods of preventing stroke comprising administering to a subject in need thereof an effective amount of an N-glycosylation inhibitor. In some embodiments, a risk of recurrent stroke is reduced in a subject. In some embodiments, a subject has previously experienced an ischemic stroke, a hemorrhagic stroke, a brainstem stroke, a transient ischemic attack, or a cryptogenic stroke. In some embodiments, the risk of a recurrent stroke comprises a risk of an ischemic stroke, a hemorrhagic stroke, a brainstem stroke, a transient ischemic attack, or a cryptogenic stroke. In some embodiments, a measurement of C-reactive protein (CRP) in the blood in a subject is reduced compared to a previous measurement of blood CRP level in the subject. In some embodiments, the measurement of CRP in the blood in a subject is reduced to below about 10 mg/L.

Described herein are methods of attenuating an ongoing ischemic event affecting the brain comprising administering to a subject in need thereof an effective amount of an N-glycosylation inhibitor. In some embodiments, the N-glycosylation inhibitor comprises hexosamine D-mannosamine (ManN), Kifunensine (Kif), Castanospermine (Cas), or a combination thereof. In some embodiments, the N-glycosylation inhibitor comprises ManN. In some embodiments, the subject is experiencing an ischemic stroke, a hemorrhagic stroke, a brainstem stroke, a transient ischemic attack, or a cryptogenic stroke. In some embodiments, the effective amount of an N-glycosylation inhibitor is administered orally, intravenously, intrathecally or intraperitoneally. In some embodiments, the effective amount of an N-glycosylation inhibitor comprises about 10 mg to about 300 g of the N-glycosylation inhibitor. In some embodiments, the effective amount of an N-glycosylation inhibitor comprises about 5 g to about 40 g of the N-glycosylation inhibitor. In some embodiments, a neurological assessment of acute stroke in a subject improves compared to a previous assessment. In some embodiments, blood flow to the brain of a subject is improved. In some embodiments, the blood flow to the brain is assessed by transcranial doppler ultrasound.

In some embodiments, the ischemic condition affecting the brain is caused by a disease or a trauma. In some embodiments, the subject is in need of inducing angiogenesis due to an ischemic condition is caused by a disease or a trauma. In some embodiments, the administration is effective to reduce ischemia in the subject. In some embodiments, the method further includes administering to the subject in need thereof an effective amount of an N-glycosylation inhibitor. In some embodiments, the method further includes administering to the subject in need thereof an effective amount of VEGF. In some embodiments, the administration is oral, intravenous, intrathecal, or intraperitoneal.

In some embodiments, the present disclosure provides pharmaceutical compositions and methods for inducing angiogenesis in a subject, including administering to the subject in need thereof an effective amount of hexosamine D-mannosamine (ManN). In some embodiments, the present disclosure provides pharmaceutical compositions and methods for inducing angiogenesis in a subject, including administering to the subject in need thereof an effective amount of Kifunensine (Kif). In some embodiments, the present disclosure provides pharmaceutical compositions and methods for inducing angiogenesis in a subject, including administering to the subject in need thereof an effective amount of Castanospermine (Cas).

In some embodiments, the administration is in vivo. In some embodiments, the subject in need of administration of a N-glycosylation inhibitor is a mammal. In some embodiments, the subject is a rodent. In some embodiments, the subject belongs to the genus Rattus. In some embodiments, the subject belongs to the genus Mus. In some embodiments, the subject belongs to the genus Canis. In some embodiments, the subject belongs to the genus Felis. In some embodiments, the subject belongs to the genus Equus. In some embodiments, the subject is a human. In some embodiments, the administration of the N-glycosylation inhibitor is effective to stimulate EC proliferation and angiogenesis. In some embodiments, the administration is effective to activate JNK signaling and an unfolded protein response caused by ER stress. In some embodiments, the administration is effective to induce changes in N-glycan and O-glycan profiles in endothelial cells.

### BRIEF DESCRIPTION OF THE DRAWINGS

This patent application contains at least one drawing executed in color. Copies of this patent or patent application with color drawing(s) will be provided by the Office upon request and payment of the necessary fec.

The novel features of the disclosure are set forth with particularity in the appended claims. A better understanding of the features and advantages of the present disclosure will be obtained by reference to the following detailed description that sets forth illustrative embodiments.

FIGS. 1A to 1F represent examples showing the effects of Mannosamine (ManN) on bovine choroidal microvascular endothelial cells (BCEC) proliferation. FIG. 1A is an image showing crystal-violet stained BCEC samples treated with ManN, in the presence or absence of VEGF. BCECs were treated with various concentrations of ManN ranging from 0.5 M to 1 mM for 5-6 days, with or without 5 ng/ml VEGF. At the end of the experiment, cells were fixed and stained with crystal violet. Cell-covered areas in various treatment groups were quantified by ImageJ software. FIG. 1B is a chart showing the effect of ManN in the presence or absence of VEGF on cell numbers. Cell numbers were quantified by adding AlamarBlue and fluorescence was measured at 530 nm/590 nm. n=3 independent samples were used. FIG. 1C is a chart showing the effect of ManN on bovine retinal microvascular endothelial cells (BREC) proliferation. n=3 biologically independent samples were used.

FIG. 1D is an image showing effects of hexosamines other than ManN on BCEC proliferation in samples. Each treatment group was tested in duplicate. FIG. 1E is a chart and images showing effect of ManN on wounded BCEC samples. BCEC confluent monolayers were scratched with 1 ml pipet tip, washed and then incubated for 40 hours in low glucose DMEM containing 1% FBS. n=3 independent samples were used. Scale bar=400 μm. Images were taken and gaps between leading wound front were quantified using AxioVision LE Rel.4.4 software. Representative images from crystal violet staining are shown. FIG. 1F is a chart showing effects of ManN in BCEC transwell migration assay. n=4 independent samples were used. Asterisks indicate a significant difference compared with control. When statistical analysis was done using a different control, a line was used between specific groups. A representative experiment is shown from 2 independent studies. Data are means+/−SD, Statistical analysis was done by 2-tailed, two-sample unequal variance t test. *p<0.05, **p<0.01.

FIGS. 2A to 2C are western blot images showing activation of ERK, AKT, mTOR, AMPKa, CREB, ACC, and eNOS is not unique to ManN. Enhanced activation of ERK (Thr 202/Tyr 204), AKT (Ser 473) and CREB (Ser 133) in BCECs following treatment with ManN together with VEGF for various times (FIG. 2A) or following pre-treatment with ManN for 8 hours, followed by VEGF stimulation for 15 minutes (FIG. 2B). For the samples shown in FIG. 2C, BCECs were treated with 40 μM ManN, ManNAc or mannose for various times. Total mTOR, ACC, eNOS, AMPKa, ERK, AKT, CREB, as well as phosphorylation of mTOR (Ser 2448), ACC (Ser 79), eNOS (Ser 1177), AMPKa (Thr 172), ERK (Thr 202/Tyr 204), AKT (Ser 473), and CREB (Ser 133) were examined by western blot analysis. f3-actin served as the loading control. Molecular weight (KDa) was labeled at the right. A representative experiment is shown from 2 independent studies.

FIGS. 3A to 3E represent examples showing that ManN specifically activates the JNK pathway in BCECs. FIG. 3A is a western blot image. BCECs grown in Growth Media (GM: low glucose DMEM containing 10% bovine calf scrum (BCS), 10 ng/ml VEGF and 5 ng/ml bFGF) were switched to growth factor-free media, followed by treatment with ManN or Mannose at 4 μM-4 mM. Four hours later, cell lysates were collected and subjected to western blot analysis for phosphorylated JNK (Thr 183/Tyr 185), p38 (Thr 180/Tyr 182) and ERK (Thr 202/Tyr 204), as well as total JNK, p38 and ERK. FIG. 3B is a western blot image showing that ManN, but not mannose, could activate JNK and its downstream c-Jun. f3-actin served as the loading control. For each study, a representative experiment is shown from 2-3 independent studies. FIG. 3C is a chart showing the effect of ManN on pre-treated samples. BCECs plated in 96-well plates were attached, pre-treated with the specific JNK inhibitor SP600125 (5 μM) for 2 hours, followed by ManN at either 40 μM or 2 mM, with or without 5 ng/ml VEGF. Six days later, cell proliferation was quantified using AlamarBluc. n=3 independent samples were used. FIG. 3D is a western blot image showing screening of siRNAs against JNKI and JNK2. 24 hours after siRNA transfection, BCECs were lysed and proteins were subjected to western blot analysis. f3-actin served as the loading control. Quantification of target knockdown is shown. FIG. 3E is a chart showing example results showing that ˜80% knockdown of JNKI and/or JNK2 by two independent siRNAs was associated with a significant reduction in the stimulatory effects of ManN on BCEC proliferation. n=3 independent samples were used. Data are means+/−SD; Asterisks indicated a significant difference compared with the control. When statistical analysis was done using a different control, a line was used between specific groups. Statistical analysis was done by 2-tailed, two-sample unequal variance t test. *p<0.05, **p<0.01.

FIGS. 4A to 4G represent examples showing that ManN affects protein glycosylation. FIG. 4A is a western blot image showing reduction of VEGFR2 molecular mass following ManN treatment. BCECs were treated with various hexosamines, their derivatives and monosaccharides at 40 μM or with VEGF at 5 ng/ml for 24 hours. VEGFR2 western blot analysis was performed. FIG. 4B is a western blot imaging showing dose-dependent effects of ManN on VEGFR2 molecular mass in BCECs. FIG. 4C is a western blot image showing Mannose could dose-dependently reverse the effect of 2 mM ManN on VEGFR2 molecular mass change, whereas mannose alone had no effect even at 10 mM.

FIG. 4D is a chart showing that 5 mM mannose could completely reverse the bell-shaped effects of ManN on BCEC proliferation with or without 5 ng/ml VEGF. BCECs plated in 96 wells were allowed to attach, followed by ManN addition. Two hours later, cells were treated with different concentrations of Mannose, with or without VEGF. Six days later, cell proliferation was quantified using AlamarBlue. n=3 independent samples were used. FIG. 4E is a western blot image showing that effects of ManN are reversible. BCECs, after treatment with 40 M ManN for 24 hours, were washed three times with low glucose DMEM. Cells were kept in low glucose DMEM for additional 8 or 24 hours. VEGFR2 western blot analysis was performed. FIG. 4F is a western blot image showing reduction of molecular mass of VEGFR2, Neuropilin-1, CD31 and c-met in HUVEC following ManN treatment at various concentrations. FIG. 4G is a western blot image showing reduction of molecular mass of VEGFR2, f31 integrin and bFGFRI in hDMVECs by ManN at various concentrations. f3-actin served as the loading control. Data are means+/−SD; Asterisks denote a significant difference compared with the control. For each study, a representative experiment is shown from 2-5 independent studies. Statistical analysis was done by 2-tailed, two-sample unequal variance t test. *p<0.05, **p<0.01.

FIGS. 5A to 5D represent examples showing that ManN specifically induces expression of unfolded protein response (UPR) responsive proteins. FIG. 5A is a western blot image. BCECs were grown in Growth Media (GM) until ˜80% confluency. Media were changed to growth factor-free media containing 10% BCS in the presence or absence of 40 or 400 μM of ManN or mannose for various times. At the end of each incubation, cell lysates were collected, proteins were separated on 4-12% Bis-Tris gel for western blot analysis. FIG. 5B is a western blot image for cells treated with various concentrations of ManN, mannose, 5 ng/ml VEGF or a combination of ManN and VEGF for 24 hours. Cell lysates were separated on NuPAGE 3-8% Tris-Acetate gel for western blot analysis. FIG. 5C is a western blot image showing that 4-PBA, but not TUDCA, could effectively block the induction of CHOP in BCECs, accompanied by a restoration of expression of transcription factor ATF-6 upon 400 μM ManN treatment. BCECs were pre-treated with 2 mM 4-PBA or 500 μM TUDCA, two chemical chaperons. Sixteen hours later, cells were switched to growth factor-free media for 4 hours in the presence of ManN. GM: Growth Media. FIG. 5D 4-PBA significantly blocked the bell-shaped effects of ManN on BCEC proliferation. Pre-treatment of cells with 1 mM 4-PBA for 8 hours abrogated additive effects of 40 μM ManN and 5 ng/ml VEGF and protected cells from toxic effect induced by 2 mM ManN. n=3 independent samples were used. For each study, a representative experiment is shown from 2-3 independent studies. Data arc means+/−SD; Asterisks indicate a significant difference compared with control. Statistical analysis was done by 2-tailed, two-sample unequal variance t test. *p<0.05, ** p<0.01.

FIGS. 6A to 6J are charts showing effects of ManN on non-endothelial cells of bovine, mouse or human origin. ManN did not promote growth of Calu6 (FIG. 6A), A673 (FIG. 6B), U87MG (FIG. 6C) and 4T1 (FIG. 6D) tumor cells. 10% FBS was used as positive control for Calu6 and A673, whereas 10 ng/ml bFGF and 1 μg/ml human apo-transferrin were used as positive controls for U87MG and 4T1, respectively. Similarly, no increases in proliferation were induced by ManN on AML12 (FIG. 6E), bovine pituitary cells (FIG. 6F), NIH3T3 cells (FIG. 6G), human RPEs (FIG. 6H), human dermal fibroblasts (FIG. 6I), and 10 human keratinocytes (FIG. 6J), alone or in combination with growth factors. Proliferation quantification was performed using AlamarBlue or MTS (for 4T1 cells). n=3 independent samples were used. Inserted in the charts in FIGS. 6A to 6J arc representative western blot analyses showing dose-dependent effects of ManN and mannose at 400 μM (2,4) and 2 mM (3,5) on bFGFRI or f31 integrin (for 4T1, AML12, NIH3T3 cells, human skeletal muscle cells, human dermal fibroblasts and human keratinocytes) molecular mass compared to the untreated control (1). f3-actin served as loading control. GM: Growth Media. Proteins were separated on NuPAGE 3-8% Tris-Acetate gel for western blot analysis. For each study, a representative experiment is shown from 2 independent studies. Asterisks indicate a significant difference compared with control. When statistical analysis was done using a different control, bracket was used between specified groups. Data were means+/−SD of the mean or an average when n=2. Statistical analysis was done by 2-tailed, two-sample unequal variance t test. *p<0.05, **p<0.01.

FIGS. 7A to 7F represent examples showing effects of protein glycosylation inhibitors on BCEC proliferation. FIG. 7A includes images of samples showing dose-dependent stimulation of BCEC proliferation by various inhibitors of glycosylation. Inhibitors were added at concentrations ranging from 0.01 to 100 μM for 3 days, with or without 5 ng/ml VEGF. At the end of the experiment, cells were fixed and stained with crystal violet. A representative experiment is shown. Kifunesine (Kif), an ERα-1,2-mannosidase I and Golgi α-mannosidase I inhibitor; Castanospermine (Cas), an a-glucosidase inhibitor. Cell-covered areas in various treatment groups were quantified by ImageJ software.

FIG. 7B is a chart showing dose-dependent effects of Kif and Cas in promoting BCEC proliferation with or without 5 ng/ml VEGF. n=3 independent samples were used. FIG. 7C includes western blot images showing that both inhibitors reduced VEGFR2 molecular mass and induced Bip expression in a dose-dependent fashion as assessed by western blot analysis. Proteins from total cell lysates were separated using 3-8% Tris-Acetate gel. BCECs were treated with various inhibitors for 24 hours. Quantification of western blots was done by densitometry. β-actin was the loading control. FIG. 7D is a chart showing acceleration of closure of monolayer gaps by Kif and Cas in BCEC scratch assay, with controls for Kif (H2O) and Cas (DMSO). Gaps were quantified using AxioVision LE Rel.4.4 software. n=3 independent samples were used. Scale bar=400 μm. FIG. 7E is a western blot image showing activation of AKT and JNK in BCECs by glycosylation inhibitors at 40 μM and VEGF at 10 ng/ml. However, Cas did not activate ERK. Quantification of phosphorylated AKT, JNK and ERK was done by densitometry analysis relative to total protein. FIG. 7F is a chart showing pre-treatment of BCECs with 5 μM SP600125 for 2 hours significantly blocked the effects of both glycosylation inhibitors on BCEC proliferation. n=3 independent samples were used. A representative experiment is shown from 2-4 independent studies. Data shown are means+/−SD. Statistical analysis was done by 2-tailed, two-sample unequal variance t test. *p<0.05, **p<0.01.

FIGS. 8A to 8D represent examples showing topical application of ManN and VEGF stimulated angiogenesis and accelerates wound healing in mice. FIG. 8A is a chart showing effect of ManN on wounds. Wounds were made on the dorsal skin of mice by 6 mm punch. VEGF and ManN each was administered daily at 20 μg per wound in 25 μL PBS for the first 4 days, with PBS as control. A 10-day wound healing study with 5 mice in each group. Wound closure rate (%) was quantified by Image J software in two independent studies. Asterisks indicated a significant difference compared with the control at each time point. FIG. 8B includes images resulting from a 4-day wound healing study with images of the wound healing process at day 1, day 2 and day 4. n=5 animals/treatment group were used. FIG. 8C includes representative images of immunohistochemical staining for CD31 in PBS control group and in VEGF and ManN combination groups (scale bar=200 μm). FIG. 8D is a chart showing quantification of CD31-positive blood vessel (red dotted circles) density around the wound areas was counted by eyes under microscopy (20× magnification). Data are means+/−SD. Statistical significance was further confirmed using Wilcoxon rank-sum test between treatment groups of interest. Asterisks indicated a significant difference compared with the PBS control. For each study, a representative experiment is shown. n=3 animals/treatment group were used. When statistical analysis was done using a different control, a line was used between specific groups. Statistical analysis was done by 2-tailed, two-sample unequal variance t test. *p<0.05, **p<0.01.

FIGS. 9A to 9D represent examples showing ManN accelerates blood perfusion recovery in a mouse ischemic hindlimb model. FIG. 9A includes images resulting from serial laser Doppler analysis of blood perfusion in hindlimbs of ManN-treated, Kif-treated and control mice. Different colors were used to indicate blood perfusion in the ischemic limb (ligated; left side) to nonischemic limb (sham; right side). Representative images at week 0 and week 1 are shown. FIG. 9B is a chart showing quantification of blood perfusion ratio between region 2 (ischemic; left limb) and region 1 (nonischemic; right limb), n=8 animals/treatment group. FIG. 9C includes images of sample tissue. Three weeks after surgery, skeletal muscle tissues were harvested and fixed. CD31 immunostaining on these tissue sections was performed to label the vasculature. H&E staining was also performed. Representative CD31 staining and H&E histological image of ischemic hindlimbs 21 days after surgery were shown. Scale bar=50 μm. FIG. 9D is a chart showing quantification of vascular density by CD31 immunostaining performed using ImageJ software, n=8 animals/treatment group, 3 independent experiments; Data are means+/−SEM. Statistical analysis was done by 2-tailed, two-sample unequal variance t test. *p<0.05, **p<0.01.

FIGS. 10A and 10B represent examples showing ManN promotes retinal neovascularization in mice. FIG. 10A includes images of tissue. Intravitreal injection of ManN increases blood vessel density. Adult mice were intravitreally injected once 500 ng of ManN, Kif or 200 ng of bFGF. PBS was used as vehicle control. Seven days after injection, PFA-fixed retinas were subjected to CD31 immunofluorescence. Representative images of CD31-positive vessels are shown. n=10 animals/treatment group, 3 independent experiments, scale bar=50 μm. FIG. 10B is a chart showing vascular density determined with ImageJ software, n=10 animals/treatment group, 3 independent experiments. Data were means+/−SEM. Statistical analysis was done by 2-tailed, two-sample unequal variance t test. *p<0.05, **p<0.01, ***p<0.001.

FIGS. 11A to 11D represent examples showing that ManN, but not structurally related molecules, stimulates endothelial cell proliferation. FIG. 11A is a chart showing additive effects of ManN and bFGF on BCEC proliferation. Bell-shaped effects of ManN on BCEC proliferation. BCECs were treated with ManN ranging from 0.4-400 μM for 5-6 days, with or without 20 ng/ml bFGF. At the end of the experiment, proliferation was quantified using AlamarBlue. FIG. 11B is a chart showing that additive effects of VEGF and ManN on BCEC proliferation are dependent on glycolysis pathway. Proliferation assays were carried out in low glucose DMEM media without growth factors or in DMEM media without glucose and pyruvate. Asterisks indicate a significant difference compared with no treatment control. Statistical analysis was also done to compare VEGF alone and VEGF plus ManN treatment groups for cells grown in two different assay media. FIGS. 11C and 11D are charts showing the effect on BCECs of various agents at 0.04 μM-5 mM in the absence (FIG. 11C) or presence (FIG. 11D) of 5 ng/ml VEGF. n=3 independent samples were used. For each study, a representative experiment is shown from 2 independent studies. Data are means+/−SD. Statistical analysis was done by 2-tailed, two-sample unequal variance t test. *p<0.05, **p<0.01.

FIGS. 12A to 12D represent experimental setup and analysis of effects of ManN treatment on infarction in the brain. FIG. 12A is a summary of the timing of the ManN treatment regimen and the timing of induced neural ischemia and later assessment of infarction. FIG. 12B demonstrates the results a first experiment of an acute middle cerebral artery occlusion (MCAO) stroke model in the mouse testing the effects of ManN treatment. FIG. 12C demonstrates the results a second experiment of an acute middle cerebral artery occlusion (MCAO) stroke model in the mouse testing the effects of ManN treatment. FIG. 12D represents a graph and analysis of the combined results of experiments in FIG. 12B and FIG. 12C.

FIGS. 13A to 13C represent analysis of effects of ManN treatment on infarction in the brain. FIG. 13A shows representative MRI coronal slices of ManN-treated and vehicle-treated mouse brains showing regions of infarction in the MCAO stroke model on Day 2 and on Day 4. FIG. 13B a graph and analysis of the combined results of infarct volumes on Day 4 of the MCAO stroke model in ManN-treated and vehicle-treated mouse brains. FIG. 13C shows TTC staining in coronal sections of ManN-treated and vehicle-treated mouse brains on Day 4 of the MCAO stroke model. FIGS. 14A to 14B represent CD31 staining and analysis of vascular density in the MCAO stroke model. FIG. 14A shows CD31 immunostaining labeling endothelial cells in ManN-treated and vehicle-treated mouse brains in regions of cerebral cortex affected by infarction on Day 6. FIG. 14B shows a graph and analysis of CD31 staining density of representative sections of cerebral cortex affected by infarction on Day 6 of vehicle-treated and ManN-treated mice indicating increase vascular density in ischemic areas of the brain following administration of ManN.

FIG. 15 shows histopathological analysis of cerebral cortex and striatum in vehicle-treated and ManN-treated mice on Day 4 in the MCAO stroke model.

### DETAILED DESCRIPTION

### Overview

All publications, patents, and patent applications mentioned in this specification are herein incorporated by reference to the same extent as if each individual publication, patent, or patent application was specifically and individually indicated to be incorporated by reference.

### Pharmaceutical Compositions

Compositions for Preventing, Attenuating, and/or Treating Stroke

Described herein are compositions for preventing, attenuating, and/or treating stroke comprising an effective amount of an N-glycosylation inhibitor administered to a subject in need thereof. In some embodiments, the N-glycosylation inhibitor comprises hexosamine D-mannosamine (ManN), Kifunensine (Kif), Castanospermine (Cas), or a combination thereof. In some embodiments, the N-glycosylation inhibitor comprises ManN and Kif. In some embodiments, the N-glycosylation inhibitor comprises ManN and Cas. In some embodiments, the N-glycosylation inhibitor comprises Kif and Cas. In some embodiments, the N-glycosylation inhibitor comprises ManN, Kif, and Cas. In some embodiments, the N-glycosylation inhibitor comprises ManN. In some embodiments, the N-glycosylation inhibitor comprises Kif. In some embodiments, the N-glycosylation inhibitor comprises Cas. In some embodiments, the composition for preventing stroke is formulated as a pharmaceutical composition. In some embodiments, the composition for attenuating stroke is formulated as a pharmaceutical composition. In some embodiments, the composition for treating stroke is formulated as a pharmaceutical composition.

In some embodiments, the subject in need of administration of a N-glycosylation inhibitor is a mammal. In some embodiments, the subject is a rodent. In some embodiments, the subject belongs to the genus Rattus. In some embodiments, the subject belongs to the genus Mus. In some embodiments, the subject belongs to the genus Canis. In some embodiments, the subject belongs to the genus Felis. In some embodiments, the subject belongs to the genus Equus. In some embodiments, the subject is a human.

In some embodiments, pharmaceutical compositions for treating stroke are for use after a stroke has occurred in a subject. In some embodiments, the subject has experienced an ischemic stroke. In some embodiments, the ischemic stroke comprises a thrombotic stroke or an embolic stroke. In some embodiments, the subject has experienced a hemorrhagic stroke. In some embodiments, the hemorrhagic stroke comprises a subarachnoid hemorrhage or an intracerebral hemorrhage. In some embodiments, the subject has experienced a brainstem stroke. In some embodiments, the subject has experienced a transient ischemic attack (TIA). In some embodiments, the subject has experienced one or more transient ischemic attacks. In some embodiments, the one or more transient ischemic attacks have occurred prior to a stroke event for which the subject is treated. A TIA may last for short period of time including not more than 1, 2, 3, 4, 5, 6, 7, 8, 9, 10, 11, 12, 13, 14, 15, 20, 25, or 30 minutes. A TIA may briefly interrupt blood supply to an area of the brain of the subject. In some embodiments, the subject experiences or has experienced onset of TIA symptoms suddenly. In some embodiments, the symptoms of TIA in the subject resolve after a period of 1, 2, 3, 4, 5, 6, 7, 8, 9, 10, 11, 12, 13, 14, 15, 20, 25, 30 minutes, or more than 30 minutes. In some embodiments, a TIA does not cause permanent damage to the brain of the subject. In some embodiments, a TIA does cause permanent damage to the brain of the subject. In some embodiments, one or more TIAs in the subject indicate a warning of elevated risk of stroke in the subject. In some embodiments, the subject is administered the pharmaceutical composition comprising an N-glycosylation inhibitor based on the previous occurrence of one or more TIAs. In some embodiments, the subject has experienced a cryptogenic stroke. In some embodiments, the subject has been diagnosed with a stroke. In some embodiments, the subject is presumed to have had a stroke. In some embodiments, the subject is suspected of having had a stroke. In some embodiments, the subject is not known to have experienced a stroke. In some embodiments, the subject is not known to have experienced a stroke, but has experienced one or more TIAs. In some embodiments, the subject is administered the pharmaceutical composition comprising one or more N-glycosylation inhibitors based on elevated risk of stroke following one or more TIAs. In some embodiments, the subject is administered the pharmaceutical composition comprising one or more N-glycosylation inhibitors based on elevated risk of recurrent stroke following one or more TIAs. In some embodiments, the subject is administered the pharmaceutical composition comprising one or more N-glycosylation inhibitors based on elevated risk of recurrent stroke following one or more previous strokes.

In some embodiments, pharmaceutical compositions for treating stroke are administered orally. In some embodiments, pharmaceutical compositions for treating stroke are administered intravenously. In some embodiments, pharmaceutical compositions for treating stroke are administered intrathecally. In some embodiments, pharmaceutical compositions for treating stroke are administered intraperitoneally. In some embodiments, pharmaceutical compositions for treating stroke are administered to the cerebrospinal fluid. In some embodiments, pharmaceutical compositions for treating stroke are administered to the choroid plexus. In some embodiments, pharmaceutical compositions for treating stroke are administered locally near a site of ischemia. In some embodiments, pharmaceutical compositions for treating stroke are administered to a blood vessel directly supplying blood to the brain. In some embodiments, pharmaceutical compositions for treating stroke are administered to a carotid artery.

### Therapeutically Effective Amounts of Pharmaceutical Compositions

In some embodiments, pharmaceutical compositions for preventing, attenuating, or treating stroke comprise an effective amount of an N-glycosylation inhibitor. In some embodiments, the pharmaceutical compositions for treating stroke comprise an effective amount of one or more N-glycosylation inhibitors. In some embodiments the N-glycosylation inhibitor comprises hexosamine D-mannosamine (ManN), Kifunensine (Kif), or Castanospermine (Cas). In some embodiments the N-glycosylation inhibitor is hexosamine D-mannosamine (ManN), Kifunensine (Kif), or Castanospermine (Cas). In some embodiments, the one or more N-glycosylation inhibitors are hexosamine D-mannosamine (ManN), Kifunensine (Kif), or Castanospermine (Cas), or any combination thereof. In some embodiments the N-glycosylation inhibitor comprises ManN. In some embodiments the N-glycosylation inhibitor comprises Kif. In some embodiments the N-glycosylation inhibitor comprises Cas. In some embodiments the N-glycosylation inhibitor is ManN. In some embodiments the N-glycosylation inhibitor is Kif. In some embodiments the N-glycosylation inhibitor is Cas. In some embodiments the N-glycosylation inhibitor consists essential of ManN. In some embodiments the N-glycosylation inhibitor consists essential of Kif. In some embodiments the N-glycosylation inhibitor consists essential of Cas. In some embodiments, an effective amount of the N-glycosylation inhibitor is at least about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, 1 g, 1.5 g, 2 g, 2.5 g, 3 g, 3.5 g, 4 g, 4.5 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 32 g, 34 g, 36 g, 38 g, 40 g, 42 g, 44 g, 46 g, 48 g, 50 g, 53 g, 56 g, 60 g, 63 g, 66 g, 70 g, 73 g, 76 g, 80 g, 85 g, 90 g, 95 g, 100 g, 105 g, 110 g, 115 g, 120 g, 125 g, 130 g, 135 g, 140 g, 145 g, 150 g, 155 g, 160 g, 165 g, 170 g, 175 g, 180 g, 185 g, 190 g, 195 g, 200 g, 215 g, 230 g, 245 g, 260 g, 275 g, 300 g, 325 g, 350 g, 375 g, 400 g, 430 g, 460 g, 500 g, 530 g, 560 g, 600 g, 630 g, 660 g, 700 g, 750 g, 800 g, 850 g, 900 g, 950 g, or 1 kg. In some embodiments, an effective amount of the N-glycosylation inhibitor is at least about 20 mg. In some embodiments, an effective amount of the N-glycosylation inhibitor is at least about 40 mg. In some embodiments, an effective amount of the N-glycosylation inhibitor is at least about 1 g. In some embodiments, an effective amount of the N-glycosylation inhibitor is at least about 2 g. In some embodiments, an effective amount of the N-glycosylation inhibitor is at least about 3 g. In some embodiments, an effective amount of the N-glycosylation inhibitor is at least about 4 g. In some embodiments, an effective amount of the N-glycosylation inhibitor is at least about 5 g. In some embodiments, an effective amount of the N-glycosylation inhibitor is at least about 6 g. In some embodiments, an effective amount of the N-glycosylation inhibitor is at least about 7 g. In some embodiments, an effective amount of the N-glycosylation inhibitor is at least about 8 g. In some embodiments, an effective amount of the N-glycosylation inhibitor is at least about 9 g. In some embodiments, an effective amount of the N-glycosylation inhibitor is at least about 10 g. In some embodiments, an effective amount of the N-glycosylation inhibitor is at least about 12 g. In some embodiments, an effective amount of the N-glycosylation inhibitor is at least about 14 g. In some embodiments, an effective amount of the N-glycosylation inhibitor is at least about 16 g. In some embodiments, an effective amount of the N-glycosylation inhibitor is at least about 20 g. In some embodiments, an effective amount of the N-glycosylation inhibitor is at least about 25 g. In some embodiments, an effective amount of the N-glycosylation inhibitor is at least about 30 g. In some embodiments, an effective amount of the N-glycosylation inhibitor is at least about 35 g. In some embodiments, an effective amount of the N-glycosylation inhibitor is at least about 40 g. In some embodiments, an effective amount of the N-glycosylation inhibitor is no more than about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, 1 g, 1.5 g, 2 g, 2.5 g, 3 g, 3.5 g, 4 g, 4.5 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 32 g, 34 g, 36 g, 38 g, 40 g, 42 g, 44 g, 46 g, 48 g, 50 g, 53 g, 56 g, 60 g, 63 g, 66 g, 70 g, 73 g, 76 g, 80 g, 85 g, 90 g, 95 g, 100 g, 105 g, 110 g, 115 g, 120 g, 125 g, 130 g, 135 g, 140 g, 145 g, 150 g, 155 g, 160 g, 165 g, 170 g, 175 g, 180 g, 185 g, 190 g, 195 g, 200 g, 215 g, 230 g, 245 g, 260 g, 275 g, 300 g, 325 g, 350 g, 375 g, 400 g, 430 g, 460 g, 500 g, 530 g, 560 g, 600 g, 630 g, 660 g, 700 g, 750 g, 800 g, 850 g, 900 g, 950 g, or 1 kg. In some embodiments, an effective amount of the N-glycosylation inhibitor is no more than about 50 g. In some embodiments, an effective amount of the N-glycosylation inhibitor is no more than about 45 g. In some embodiments, an effective amount of the N-glycosylation inhibitor is no more than about 40 g. In some embodiments, an effective amount of the N-glycosylation inhibitor is no more than about 35 g. In some embodiments, an effective amount of the N-glycosylation inhibitor is no more than about 30 g. In some embodiments, an effective amount of the N-glycosylation inhibitor is no more than about 25 g. In some embodiments, an effective amount of the N-glycosylation inhibitor is no more than about 20 g. In some embodiments, an effective amount of the N-glycosylation inhibitor is no more than about 16 g. In some embodiments, an effective amount of the N-glycosylation inhibitor is no more than about 14 g. In some embodiments, an effective amount of the N-glycosylation inhibitor is no more than about 12 g. In some embodiments, an effective amount of the N-glycosylation inhibitor is no more than about 10 g. In some embodiments, an effective amount of the N-glycosylation inhibitor is no more than about 8 g. In some embodiments, an effective amount of the N-glycosylation inhibitor is no more than about 6 g. In some embodiments, an effective amount of the N-glycosylation inhibitor is no more than about 4 g. In some embodiments, an effective amount of the N-glycosylation inhibitor is no more than about 2 g. In some embodiments, an effective amount of the N-glycosylation inhibitor is no more than about 1 g. In some embodiments, an effective amount of the N-glycosylation inhibitor is no more than about 100 mg. In some embodiments, an effective amount of the N-glycosylation inhibitor is no more than about 50 mg. In some embodiments, an effective amount of the N-glycosylation inhibitor is no more than about 40 mg. In some embodiments an effective amount of an N-glycosylation inhibitor is between about 1 mg to about 10 mg, about 3 mg to about 10 mg, about 5 mg to about 15 mg, about 10 mg to about 20 mg, about 15 mg to about 45 mg, about 20 mg to about 55 mg, about 25 mg to about 70 mg, about 35 mg to about 75 mg, about 40 mg to about 105 mg, about 45 mg to about 85 mg, about 50 mg to about 80 mg, about 55 about 125 mg, about 60 mg to about 150 mg, about 65 mg to about 185 mg, about 70 mg to about 125 mg, about 75 mg to about 200 mg, about 80 mg to about 225 mg, about 85 mg to about 300 mg, about 90 mg to about 180 mg, about 100 mg to about 150 mg, about 100 mg to about 225 mg, about 100 mg to about 350 mg, about 100 mg to about 500 mg, about 110 mg to about 200 mg, about 110 mg to about 275 mg, about 150 mg to about 375 mg, about 175 mg to about 500 mg, about 190 mg to about 315 mg, about 225 mg to about 600 mg, about 250 mg to about 700 mg, about 250 mg to about 500 mg, about 260 mg to about 800 mg, about 275 mg to about 600 mg, about 300 mg to about 900 mg, about 350 mg to about 750 mg, about 400 mg to about 2 g, about 425 mg to about 1.1 g, about 450 mg to about 1.5 g, about 500 mg to about 850 mg, about 525 mg to about 1.3 g, about 600 mg to about 2 g, about 700 mg to about 3 g, about 800 mg to about 4 g, about 900 mg to about 5 g, about 1 g to about 3 g, about 1.1 g to about 6 g, about 1.2 g to about 8 g, about 1.4 g to about 10 g, about 2.5 g to about 7.5 g, about 3 g to about 12 g, about 4 g to about 15 g, about 4.5 g to about 16 g, about 5 g to about 20 g, about 6 g to about 18 g, about 7 g to about 21 g, about 8 g to about 23 g, about 9 g to about 25 g, about 10 g to about 30 g, about 12 g to about 35 g, about 14 g to about 28 g, about 15 g to about 45 g, about 18 g to about 36 g, about 20 g to about 50 g, about 25 g to about 65 g, about 30 g to about 70 g, about 35 g to about 80 g, about 40 g to about 100 g, about 45 g to about 115 g, about 50 g to about 135 g, about 20 g to about 300 g, about 20 g to about 500 g, about 25 g to about 800 g, about 70 g to about 385 g, about 75 g to about 450 g about 85 g to about 600 g, about 100 g to about 800 g, about 200 g to about 1 kg, or about 475 g to about 1 kg. In some embodiments an effective amount of the N-glycosylation inhibitor is between about 20 mg to about 60 mg. In some embodiments an effective amount of the N-glycosylation inhibitor is between about 1 g to about 60 g. In some embodiments an effective amount of the N-glycosylation inhibitor is between about 1 g to about 30 g. In some embodiments an effective amount of the N-glycosylation inhibitor is between about 1 g to about 15 g. In some embodiments an effective amount of the N-glycosylation inhibitor is between about 3 g to about 30 g. In some embodiments an effective amount of the N-glycosylation inhibitor is between about 3 g to about 15 g. In some embodiments an effective amount of the N-glycosylation inhibitor is between about 5 g to about 50 g. In some embodiments an effective amount of the N-glycosylation inhibitor is between about 5 g to about 30 g. In some embodiments an effective amount of the N-glycosylation inhibitor is between about 5 g to about 20 g. In some embodiments an effective amount of the N-glycosylation inhibitor is between about 5 g to about 15 g. In some embodiments an effective amount of the N-glycosylation inhibitor is between about 5.8 g to about 7.3 g. In some embodiments an effective amount of the N-glycosylation inhibitor is between about 7.8 g to about 10 g. In some embodiments an effective amount of the N-glycosylation inhibitor is between about 11.8 g to about 14.8 g. In some embodiments an effective amount of the N-glycosylation inhibitor is between about 23.7 g to about 29.7 g. In some embodiments an effective amount of the N-glycosylation inhibitor is between about 31.7 g to about 39.6 g.

### Pro-Angiogenic Factors

In some embodiments described herein, are pharmaceutical compositions comprising a pro-angiogenic factor that when combined with a therapeutically effective amount of an N-glycosylation inhibitor yield a synergistic increase in angiogenic growth. In some embodiments, the pro-angiogenic factor when combined with a therapeutically effective amount of an N-glycosylation inhibitor yield a synergistic increase in endothelial proliferation. In some embodiments, the pro-angiogenic factor when combined with a therapeutically effective amount of an N-glycosylation inhibitor yield a synergistic increase in angiogenic growth without a significant amount of vascular leakage or vascular edema. In some embodiments, the pro-angiogenic factor comprises VEGF. In some embodiments, the VEGF comprises VEGF-A, VEGF-B, VEGF-C, VEGF-D, placental growth factor (PLGF), or a combination thereof. In some embodiments, the VEGF comprises a recombinant VEGF. In some embodiments, the VEGF is administered locally near a site in the brain of a subject affected by an ischemic event. In some embodiments, the VEGF is administered systemically. In some embodiments, the VEGF is administered intravenously. In some embodiments, the VEGF is administered intrathecally. In some embodiments, the VEGF is administered to the cerebrospinal fluid. In some embodiments, the VEGF is administered to the choroid plexus. In some embodiments, the pro-angiogenic factor is formulated in a pharmaceutical composition separately from the N-glycosylation inhibitor. In some embodiments, the pro-angiogenic factor is formulated in a pharmaceutical composition together with the N-glycosylation inhibitor.

### Pharmaceutical Formulations

Described herein are pharmaceutical compositions for preventing, attenuating, and/or treating stroke comprising an effective amount of an N-glycosylation inhibitor administered to a subject in need thereof. In some embodiments, the pharmaceutical composition for preventing, attenuating, and/or treating stroke comprises an effective amount of one ore more N-glycosylation inhibitors administered to a subject in need thereof. In some embodiments, the one or more N-glycosylation inhibitors comprises ManN, Kif, or Cas, or any combination thereof. In some embodiments, the one or more N-glycosylation inhibitors comprises a compound having a chemical structural similar to ManN. In some embodiments, the one or more N-glycosylation inhibitors having a chemical structural similar to ManN is listed in Table 1. In some embodiments, the N-glycosylation inhibitor administered to a subject comprises ManN. In some embodiments, the N-glycosylation inhibitor administered to a subject comprises ManN and one or more compounds listed in Table 1. In some embodiments, the N-glycosylation inhibitor administered to a subject comprises one or more compounds listed in Table 1. In some embodiments, the N-glycosylation inhibitor administered to a subject consists essentially of ManN. In some embodiments, the N-glycosylation inhibitor administered to a subject consists of ManN. In some embodiments, the pharmaceutical composition comprises a therapeutically effect amount of one or more N-glycosylation inhibitors dissolved in water. In some embodiments, the pharmaceutical composition comprises. In some embodiments, the pharmaceutical composition comprises a therapeutically effect amount of one or more N-glycosylation inhibitors formulated as a pharmaceutical solution. In some embodiments, the pharmaceutical solution is a liquid preparation in which the one or more N-glycosylation inhibitors and one or more pharmaceutically acceptable excipients are dissolved in a chosen solvent system. In some embodiments, pharmaceutically acceptable excipient is purified water. In some embodiments, a vehicle solution is purified water. In some embodiments, the pharmaceutical solution comprises a co-solvent. In some embodiments, the co-solvent is propylene glycol, glycerol, propylene glycol, glycerin, poly(ethylene glycol), or ethanol. In some embodiments, the pharmaceutical solution comprises one or more agents specifically to enhance the solubility of the N-glycosylation inhibitor in the vehicle. In some embodiments, the one or more agents specifically to enhance the solubility of the N-glycosylation inhibitor in the vehicle are surface-active agents. In some embodiments, the pharmaceutical solution comprises one or more preservatives. In some embodiments, the one or more preservatives are parahydroxybenzoate esters (methylhydroxybenzoate and propylhydroxybenzoate), boric acid and borate salts, sorbic acid and sorbate salts, a phenolic, or any combination thereof. In some embodiments, benzoic acid and salts (0.1-0.3%) are used as preservatives. In some embodiments, sorbic acid and its salts (0.05-0.2%) are used as preservatives. In some embodiments, alkyl esters of parahydroxybenzoic acid (0.001-0.2%) are used as preservatives. In some embodiments, methyl and propyl parahydroxybenzoates (in a ratio of 9:1) are used as preservatives. In some embodiments, the pharmaceutical solution comprises one or more viscosity modifiers. In some embodiments, the one or more viscosity modifiers are hydrophilic polymers such as cellulose derivatives (e.g., methylcellulose, hydroxyethylcellulose, or hydroxypropylcellulose), alginic acid, sodium alginate, and/or polyvinylpyrrolidone. In some embodiments, the pharmaceutical solution comprises one or more antioxidants. In some embodiments, the one or more antioxidants are sodium formaldehyde sulphoxylate, butylated hydroxyanisole, butylated hydroxytoluene, or any combination thereof. In some embodiments, the pharmaceutical solution comprises one or more coloring agents. In some embodiments, the pharmaceutical solution comprises one or more flavoring agents. In some embodiments, the pharmaceutical solution comprises one or more sweetening agents. In some embodiments, the one or more sweetening agents are aspartame, sucralose, acesulfame K, saccharin, xylitol, sucrose, liquid glucose, glycerol, sorbitol, or any combination thereof. In some embodiments, the one or more sweetening agents are added to increase the palatability of the one or more N-glycosylation inhibitors. In some embodiments, the pharmaceutical solution comprises one or more buffering systems to regulate the pH of the formulation. In some embodiments, the pH of the pharmaceutical solution is between about pH 5.0-8.0. In some embodiments, the pH of the pharmaceutical solution is between about pH 6.0-7.5. In some embodiments, the pH of the pharmaceutical solution is selected to optimize solubility of the one or more N-glycosylation inhibitors. In some embodiments, the pharmaceutical composition comprises a therapeutically effect amount of one or more N-glycosylation inhibitors formulated as a pharmaceutical oral suspension comprising a suspending agents and one or more of vehicle, solvent, co-solvent, preservative, sweetener, anti-foaming agent, wetting agent, buffering agent, and flavoring agent. In some embodiments, the choice of formulation of the pharmaceutical composition is dictated by the route of administration by which the one or more N-glycosylation inhibitors are administered to the subject.

In some embodiments, pharmaceutical compositions for preventing, attenuating, and/or treating stroke comprising an effective amount of one or more N-glycosylation inhibitors administered to a subject in need thereof are formulated with the one or more N-glycosylation inhibitors dissolved in water for oral administration. In some embodiments, the one or more N-glycosylation inhibitors are formulated as a concentration of about 0.01%, 0.02%, 0.03%, 0.04%, 0.05%, 0.075%, 0.1%, 0.2%, 0.4%, 0.6%, 0.8%, 1.0%, 1.25%, 1.5%, 1.75%, 2%, 3%, 4%, 5%, 6%, 7%, 8%, 9%, 10%, 12%, 14%, 15%, 16%, 17%, 18%, 19%, 20%, 21%, 22%, 23%, 24%, 25%, 30%, 35%, 40%, 45%, or 50% dissolved in water. In some embodiments, the pharmaceutical composition for preventing, attenuating, and/or treating stroke comprises ManN dissolved in purified water. In some embodiments, the ManN is dissolved in purified water at a concentration of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50%. In some embodiments, the ManN is dissolved in purified water at a concentration of about 20%. In some embodiments, the pharmaceutical composition for preventing, attenuating, and/or treating stroke comprising an effective amount of one or more N-glycosylation inhibitors administered to a subject in need thereof comprises a pharmaceutical solution of 20% ManN dissolved in water. In some embodiments, the pharmaceutical composition for preventing, attenuating, and/or treating stroke is used in a method comprising administering to the subject in need thereof a therapeutically effective amount of a pharmaceutical solution comprising about 20% ManN dissolved in water. In some embodiments, the pharmaceutical composition for preventing, attenuating, and/or treating stroke is used in a method comprising administering to the subject in need thereof a therapeutically effective amount of a pharmaceutical solution, wherein the pharmaceutical solution consists essentially of about 20% ManN dissolved in water. In some embodiments, the pharmaceutical composition for preventing, attenuating, and/or treating stroke is used in a method comprising administering to the subject in need thereof a therapeutically effective amount of a pharmaceutical solution, wherein the pharmaceutical solution consists of about 20% ManN dissolved in water. In some embodiments, a pharmaceutical solution for use in a method for preventing, attenuating, and/or treating stroke comprises a therapeutically effective amount of ManN dissolved in a beverage for oral administration. In some embodiments, a therapeutically effective amount of ManN is dissolved in water for oral administration for use in a method of preventing, attenuating, and/or treating stroke. In some embodiments, a therapeutically effective amount of ManN is dissolved in milk for oral administration for use in a method of preventing, attenuating, and/or treating stroke. In some embodiments, a therapeutically effective amount of ManN is dissolved in a protein nutrition shake (e.g., Ensure®) for oral administration for use in a method of preventing, attenuating, and/or treating stroke. In some embodiments, a therapeutically effective amount of ManN is dissolved in a balanced electrolyte beverage (e.g., Pedialyte® or Pedialyte AdvancedCare® Plus) for oral administration for use in a method of preventing, attenuating, and/or treating stroke.

### Methods

### Methods of Treating Stroke

Described herein are method of treating stroke comprising administering to a subject in need thereof an effective amount of an N-glycosylation inhibitor. In some embodiments, the N-glycosylation inhibitor comprises hexosamine D-mannosamine (ManN), Kifunensine (Kif), Castanospermine (Cas), or a combination thereof. In some embodiments, the N-glycosylation inhibitor comprises ManN and Kif. In some embodiments, the N-glycosylation inhibitor comprises ManN and Cas. In some embodiments, the N-glycosylation inhibitor comprises Kif and Cas. In some embodiments, the N-glycosylation inhibitor comprises ManN, Kif, and Cas. In some embodiments, the N-glycosylation inhibitor comprises ManN. In some embodiments, the N-glycosylation inhibitor comprises Kif. In some embodiments, the N-glycosylation inhibitor comprises Cas. In some embodiments, method of treating stroke comprising administering to a subject in need thereof an effective amount of one or more N-glycosylation inhibitors. In some embodiments the N-glycosylation inhibitor comprises hexosamine D-mannosamine (ManN), Kifunensine (Kif), or Castanospermine (Cas). In some embodiments the N-glycosylation inhibitor is hexosamine D-mannosamine (ManN), Kifunensine (Kif), or Castanospermine (Cas). In some embodiments, the one or more N-glycosylation inhibitors are hexosamine D-mannosamine (ManN), Kifunensine (Kif), or Castanospermine (Cas), or any combination thereof. In some embodiments the N-glycosylation inhibitor comprises ManN. In some embodiments the N-glycosylation inhibitor comprises Kif. In some embodiments the N-glycosylation inhibitor comprises Cas. In some embodiments the N-glycosylation inhibitor is ManN. In some embodiments the N-glycosylation inhibitor is Kif. In some embodiments the N-glycosylation inhibitor is Cas. In some embodiments the N-glycosylation inhibitor consists essential of ManN. In some embodiments the N-glycosylation inhibitor consists essential of Kif. In some embodiments the N-glycosylation inhibitor consists essential of Cas. In some embodiments, the one or more N-glycosylation inhibitors comprises a compound having a chemical structural similar to ManN. In some embodiments, the one or more N-glycosylation inhibitors having a chemical structural similar to ManN is listed in Table 1. In some embodiments, the N-glycosylation inhibitor administered to a subject comprises ManN. In some embodiments, the N-glycosylation inhibitor administered to a subject comprises ManN and one or more compounds listed in Table 1. In some embodiments, the N-glycosylation inhibitor administered to a subject comprises one or more compounds listed in Table 1. In some embodiments, the N-glycosylation inhibitor is formulated in a pharmaceutical composition.

In some embodiments, the subject in need of administration of a N-glycosylation inhibitor is a mammal. In some embodiments, the subject is a rodent. In some embodiments, the subject belongs to the genus Rattus. In some embodiments, the subject belongs to the genus Mus. In some embodiments, the subject belongs to the genus Canis. In some embodiments, the subject belongs to the genus Felis. In some embodiments, the subject belongs to the genus Equus. In some embodiments, the subject is a human.

In some embodiments, methods of treating stroke are for use after a stroke has occurred in a subject. In some embodiments, the subject has experienced an ischemic stroke. In some embodiments, the ischemic stroke comprises a thrombotic stroke or an embolic stroke. In some embodiments, the subject has experienced a hemorrhagic stroke. In some embodiments, the hemorrhagic stroke comprises a subarachnoid hemorrhage or an intracerebral hemorrhage. In some embodiments, the subject has experienced a brainstem stroke. In some embodiments, the subject has experienced a transient ischemic attack (TIA). In some embodiments, the subject has experienced a cryptogenic stroke. In some embodiments, the subject has been diagnosed with a stroke. In some embodiments, the subject is presumed to have had a stroke. In some embodiments, the subject is suspected of having had a stroke. In some embodiments, the subject has experienced one or more transient ischemic attacks. In some embodiments, the one or more transient ischemic attacks have occurred prior to a stroke event for which the subject is treated. A TIA may last for short period of time including not more than 1, 2, 3, 4, 5, 6, 7, 8, 9, 10, 11, 12, 13, 14, 15, 20, 25, or 30 minutes. A TIA may briefly interrupt blood supply to an area of the brain of the subject. In some embodiments, the subject experiences or has experienced onset of TIA symptoms suddenly. In some embodiments, the symptoms of TIA in the subject resolve after a period of 1, 2, 3, 4, 5, 6, 7, 8, 9, 10, 11, 12, 13, 14, 15, 20, 25, 30 minutes, or more than 30 minutes. In some embodiments, a TIA does not cause permanent damage to the brain of the subject. In some embodiments, a TIA does cause permanent damage to the brain of the subject. In some embodiments, one or more TIAs in the subject indicate a warning of elevated risk of stroke in the subject. In some embodiments, the subject is administered the pharmaceutical composition comprising an N-glycosylation inhibitor based on the previous occurrence of one or more TIAs.

In some embodiments described herein are methods for treating stroke wherein a pharmaceutical composition comprising an effective amount of an N-glycosylation inhibitor is administered via various routes of administration. In some embodiments, pharmaceutical compositions for treating stroke are administered intravenously. In some embodiments, pharmaceutical compositions for treating stroke are administered intrathecally. In some embodiments, pharmaceutical compositions for treating stroke are administered intraperitoneally. In some embodiments, pharmaceutical compositions for treating stroke are administered to the cerebrospinal fluid. In some embodiments, pharmaceutical compositions for treating stroke are administered to the choroid plexus. In some embodiments, pharmaceutical compositions for treating stroke are administered locally near a site of ischemia. In some embodiments, pharmaceutical compositions for treating stroke are administered to a blood vessel directly supplying blood to the brain. In some embodiments, pharmaceutical compositions for treating stroke are administered to a carotid artery.

In some embodiments described herein are methods for treating stroke wherein a pharmaceutical composition comprising an effective amount of an N-glycosylation inhibitor is administered according to a treatment regimen. In some embodiments, the treatment regimen comprises a single dosing treatment regimen. In some embodiments, single dosing treatment regimen comprises a pretreatment regimen. In some embodiments, single dosing treatment regimen comprises a concurrent treatment regimen. In some embodiments, single dosing treatment regimen comprises a post-treatment regimen. In some embodiments, the treatment regimen comprises a multiple dosing treatment regimen. In some embodiments, multiple dosing treatment regimen comprises a pretreatment regimen. In some embodiments, multiple dosing treatment regimen comprises a concurrent treatment regimen. In some embodiments, multiple dosing treatment regimen comprises a post-treatment regimen. In some embodiments multiple dosing treatment regimen comprises a pretreatment and a post-treatment regimen. In some embodiments multiple dosing treatment regimen comprises a pretreatment and a concurrent regimen. In some embodiments multiple dosing treatment regimen comprises a concurrent and a post-treatment regimen. In some embodiments multiple dosing treatment regimen comprises a pretreatment, a concurrent, and a post-treatment regimen. In some embodiments, the multiple treatment regimens comprise 2 or more treatments, 3 or more treatments, 4 or more treatments, 5 or more treatments, 6 or more treatments, 7 or more treatments, 8 or more treatments, 9 or more treatments, 10 or more treatments, 12 or more treatments, 15 or more treatments, 20 or more treatments, or 25 or more treatments. In some embodiments, a multiple treatment regimen will comprise between 1-2 treatments, between 1-3 treatments, between 1-4 treatments, between 1-5 treatments, between 1-6 treatments, between 1-7 treatments, between 1-8 treatments, between 1-9 treatments, between 1-10 treatments, between 1-12 treatments, between 1-15 treatments, between 1-20 treatments, between 2-3 treatments, between 2-4 treatments, between 2-5 treatments, between 2-6 treatments, between 2-7 treatments, between 2-8 treatments, between 2-9 treatments, between 2-10 treatments, between 2-12 treatments, between 2-15 treatments, between 2-25 treatments, between 3-4 treatments, between 3-5 treatments, between 3-6 treatments, between 3-7 treatments, between 3-8 treatments, between 3-9 treatments, between 3-10 treatments, between 3-14 treatments, between 3-18 treatments, between 3-30 treatments, between 4-5 treatments, between 4-6 treatments, between 4-7 treatments, between 4-8 treatments, between 4-9 treatments, between 4-10 treatments, between 4-15 treatments, between 5-6 treatments, between 5-7 treatments, between 5-8 treatments, between 5-10 treatments, between 5-14 treatments, between 5-23 treatments, between 5-34 treatments, between 6-7 treatments, between 6-8 treatments, between 6-9 treatments, between 6-10 treatments, between 6-12 treatments, between 6-17 treatments, between 6-22 treatments, between 6-30 treatments, between 7-8 treatments, between 7-9 treatments, between 7-12 treatments, between 7-18 treatments, between 9-18 treatments, between 10-20 treatments, between 12-25 treatments, between 14-35 treatments, between 15-50 treatments, or between 20-100 treatments. In some embodiments, a pretreatment regimen comprises one or more treatments comprising administering a pharmaceutical composition described herein to the subject wherein the pretreatments begin following a determination that the subject is at increased risk of suffering from a stroke. In some embodiments, a pretreatment regimen comprises one or more treatments comprising administering a pharmaceutical composition described herein to the subject wherein the pretreatments begin following a determination that the subject is at increased risk of suffering from a recurrence of a stroke. In some embodiments, a pretreatment regimen comprises one or more treatments comprising administering a pharmaceutical composition described herein to the subject wherein the pretreatments begin following one or more TIAs in the subject. In some embodiments, a pretreatment regimen comprises one or more pretreatments comprising administering a pharmaceutical composition described herein to the subject wherein the one or more pretreatments begin at least about 48, 44, 40, 36, 32, 28, 24, 22, 20, 18, 16, 14, 12, 10, 8, 6, 5, 4, 3, 2, or 1 hours prior to a stroke event. In some embodiments, the concurrent regimen comprises one or more concurrent treatments comprising administering a pharmaceutical composition described herein to the subject wherein the one or more concurrent treatments begin within about 0.1, 0.2, 0.3, 0.4, 0.5, 0.75, 1, 1.25, 1.5, 1.75, 2, 2.5, 3, 3.5, 4, 4.5, 5, 5.5, 6, 6.5, 7, 8, 9, 10, 11, 12, 13, 14, 15, 16, 17, 18, 20, 22, 24, 28, 32, 36, 40, 44, or 48 hours following an estimated beginning of an initiation time of a stroke event in the subject. In some embodiments, the post-treatment regimen comprises one or more post-treatments comprising administering a pharmaceutical composition described herein to the subject wherein the one or more post-treatments begin about 0.1, 0.2, 0.3, 0.4, 0.5, 0.75, 1, 1.25, 1.5, 1.75, 2, 2.5, 3, 3.5, 4, 4.5, 5, 5.5, 6, 6.5, 7, 8, 9, 10, 11, 12, 13, 14, 15, 16, 17, 18, 20, 22, 24, 28, 32, 36, 40, 44, 46, 48, 52, 56, 60, 64, 68, 72, 84, 96, 108, or 120 hours following a restoration of approximately normative blood flood to the a region of the brain of the subject affected by a recent stroke event. In some embodiments, the method comprises the subject undergoing a pretreatment regimen and a post-treatment regimen. In some embodiments, the method comprises the subject undergoing a pretreatment regimen and a concurrent treatment regimen. In some embodiments, the method comprises the subject undergoing a pretreatment regimen, a concurrent treatment regimen, and a post-treatment regimen. In some embodiments, the method comprises the subject undergoing a concurrent treatment regimen and a post-treatment regimen. In some embodiments, the method comprises the subject undergoing a post-treatment regimen. In some embodiment the pretreatment regimen, the concurrent treatment regimen, or the post-treatment regimen comprises administering a pharmaceutical composition described herein to the subject at a determined dosage with a frequency of one administration about every 10 min, 20 min, 30 min, 45 min, 1 hr, 1.5 hr, 2 hr, 2.5 hr, 3 hr, 4 hr, 5 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 52 hr, 56 hr, 60 hr, 64 hr, 68 hr, 72 hr, 3.5 days, 4 days, 5 days, 6 days, 7 days, 8 days, 9 days, 10 days, 11 days, 12 days, 13 days, 14 days, 15 days, 18 days, 20 days, 21 days, 24 days, 28 days, or 30 days. In some embodiments, the post-treatment regimen comprises administering a pharmaceutical composition described herein to the subject at a determined dosage with a frequency of one administration about every other day. In some embodiments, the post-treatment regimen comprises administering a pharmaceutical composition described herein to the subject at a determined dosage with a frequency of one administration about every other day until one or more symptoms of stroke in the subject have shown improvement or have resolved completely. In some embodiments, the method comprises initiating administering of a pharmaceutical composition described herein to the subject within about 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 48, 52, 56, 60, 64, 68, 72, 84, 96, or 120 hours after an ischemic event. In some embodiments, the method comprises initiating administering of a pharmaceutical composition described herein to the subject within about 1 hour to about 12 hours after an ischemic event. In some embodiments, the method comprises initiating administering of a pharmaceutical composition described herein to the subject within about 2 hour to about 6 hours after an ischemic event. In some embodiments, the method comprises initiating administering of a pharmaceutical composition described herein to the subject within about 2 hour to about 48 hours after an ischemic event. In some embodiments, the method comprises initiating administering of a pharmaceutical composition described herein to the subject within about 2 hour to about 24 hours after an ischemic event.

In some embodiments described herein, methods of single dose treatment regimens comprise administering a therapeutically effective amount of one or more N-glycosylation inhibitors described herein. In some embodiments, the one or more N-glycosylation inhibitors comprise ManN, Kif, Cas, or any combination thereof. In some embodiments, the one or more N-glycosylation inhibitors comprise ManN. In some embodiments, the one or more N-glycosylation inhibitors comprise Kif. In some embodiments, the one or more N-glycosylation inhibitors comprise Cas. In some embodiments, the one or more N-glycosylation inhibitors consists essentially of ManN. In some embodiments, the one or more N-glycosylation inhibitors is listed in Table 1. In some embodiments, an effective amount of the N-glycosylation inhibitor is at least about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, 1 g, 1.5 g, 2 g, 2.5 g, 3 g, 3.5 g, 4 g, 4.5 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 32 g, 34 g, 36 g, 38 g, 40 g, 42 g, 44 g, 46 g, 48 g, 50 g, 53 g, 56 g, 60 g, 63 g, 66 g, 70 g, 73 g, 76 g, 80 g, 85 g, 90 g, 95 g, 100 g, 105 g, 110 g, 115 g, 120 g, 125 g, 130 g, 135 g, 140 g, 145 g, 150 g, 155 g, 160 g, 165 g, 170 g, 175 g, 180 g, 185 g, 190 g, 195 g, 200 g, 215 g, 230 g, 245 g, 260 g, 275 g, 300 g, 325 g, 350 g, 375 g, 400 g, 430 g, 460 g, 500 g, 530 g, 560 g, 600 g, 630 g, 660 g, 700 g, 750 g, 800 g, 850 g, 900 g, 950 g, or 1 kg. In some embodiments, an effective amount of the N-glycosylation inhibitor is at least about 40 mg. In some embodiments, an effective amount of the N-glycosylation inhibitor is at least about 1 g. In some embodiments, an effective amount of the N-glycosylation inhibitor is at least about 2 g. In some embodiments, an effective amount of the N-glycosylation inhibitor is at least about 3 g. In some embodiments, an effective amount of the N-glycosylation inhibitor is at least about 4 g. In some embodiments, an effective amount of the N-glycosylation inhibitor is at least about 5 g. In some embodiments, an effective amount of the N-glycosylation inhibitor is at least about 6 g. In some embodiments, an effective amount of the N-glycosylation inhibitor is at least about 7 g. In some embodiments, an effective amount of the N-glycosylation inhibitor is at least about 8 g. In some embodiments, an effective amount of the N-glycosylation inhibitor is at least about 9 g. In some embodiments, an effective amount of the N-glycosylation inhibitor is at least about 10 g. In some embodiments, an effective amount of the N-glycosylation inhibitor is at least about 11 g. In some embodiments, an effective amount of the N-glycosylation inhibitor is at least about 12 g. In some embodiments, an effective amount of the N-glycosylation inhibitor is at least about 13 g. In some embodiments, an effective amount of the N-glycosylation inhibitor is at least about 14 g. In some embodiments, an effective amount of the N-glycosylation inhibitor is at least about 15 g. In some embodiments, an effective amount of the N-glycosylation inhibitor is at least about 18 g. In some embodiments, an effective amount of the N-glycosylation inhibitor is at least about 20 g. In some embodiments, an effective amount of the N-glycosylation inhibitor is at least about 22 g. In some embodiments, an effective amount of the N-glycosylation inhibitor is at least about 25 g. In some embodiments, an effective amount of the N-glycosylation inhibitor is at least about 27 g. In some embodiments, an effective amount of the N-glycosylation inhibitor is at least about 30 g. In some embodiments, an effective amount of the N-glycosylation inhibitor is at least about 35 g. In some embodiments, an effective amount of the N-glycosylation inhibitor is at least about 40 g. In some embodiments, an effective amount of the N-glycosylation inhibitor is no more than about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, 1 g, 1.5 g, 2 g, 2.5 g, 3 g, 3.5 g, 4 g, 4.5 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 32 g, 34 g, 36 g, 38 g, 40 g, 42 g, 44 g, 46 g, 48 g, 50 g, 53 g, 56 g, 60 g, 63 g, 66 g, 70 g, 73 g, 76 g, 80 g, 85 g, 90 g, 95 g, 100 g, 105 g, 110 g, 115 g, 120 g, 125 g, 130 g, 135 g, 140 g, 145 g, 150 g, 155 g, 160 g, 165 g, 170 g, 175 g, 180 g, 185 g, 190 g, 195 g, 200 g, 215 g, 230 g, 245 g, 260 g, 275 g, 300 g, 325 g, 350 g, 375 g, 400 g, 430 g, 460 g, 500 g, 530 g, 560 g, 600 g, 630 g, 660 g, 700 g, 750 g, 800 g, 850 g, 900 g, 950 g, or 1 kg. In some embodiments, an effective amount of the N-glycosylation inhibitor is no more than about 40 g. In some embodiments, an effective amount of the N-glycosylation inhibitor is no more than about 35 g. In some embodiments, an effective amount of the N-glycosylation inhibitor is no more than about 30 g. In some embodiments, an effective amount of the N-glycosylation inhibitor is no more than about 25 g. In some embodiments, an effective amount of the N-glycosylation inhibitor is no more than about 20 g. In some embodiments, an effective amount of the N-glycosylation inhibitor is no more than about 18 g. In some embodiments, an effective amount of the N-glycosylation inhibitor is no more than about 15 g. In some embodiments, an effective amount of the N-glycosylation inhibitor is no more than about 12 g. In some embodiments, an effective amount of the N-glycosylation inhibitor is no more than about 10 g. In some embodiments, an effective amount of the N-glycosylation inhibitor is no more than about 8 g. In some embodiments, an effective amount of the N-glycosylation inhibitor is no more than about 6 g. In some embodiments, an effective amount of the N-glycosylation inhibitor is no more than about 4 g. In some embodiments, an effective amount of the N-glycosylation inhibitor is no more than about 2 g. In some embodiments, an effective amount of the N-glycosylation inhibitor is no more than about 1 g. In some embodiments, an effective amount of the N-glycosylation inhibitor is no more than about 40 mg. In some embodiments an effective amount of the N-glycosylation inhibitor is between about 1 mg to about 10 mg, about 3 mg to about 10 mg, about 5 mg to about 15 mg, about 10 mg to about 20 mg, about 15 mg to about 45 mg, about 20 mg to about 55 mg, about 25 mg to about 70 mg, about 35 mg to about 75 mg, about 40 mg to about 105 mg, about 45 mg to about 85 mg, about 50 mg to about 80 mg, about 55 about 125 mg, about 60 mg to about 150 mg, about 65 mg to about 185 mg, about 70 mg to about 125 mg, about 75 mg to about 200 mg, about 80 mg to about 225 mg, about 85 mg to about 300 mg, about 90 mg to about 180 mg, about 100 mg to about 150 mg, about 100 mg to about 225 mg, about 100 mg to about 350 mg, about 100 mg to about 500 mg, about 110 mg to about 200 mg, about 110 mg to about 275 mg, about 150 mg to about 375 mg, about 175 mg to about 500 mg, about 190 mg to about 315 mg, about 225 mg to about 600 mg, about 250 mg to about 700 mg, about 250 mg to about 500 mg, about 260 mg to about 800 mg, about 275 mg to about 600 mg, about 300 mg to about 900 mg, about 350 mg to about 750 mg, about 400 mg to about 2 g, about 425 mg to about 1.1 g, about 450 mg to about 1.5 g, about 500 mg to about 850 mg, about 525 mg to about 1.3 g, about 600 mg to about 2 g, about 700 mg to about 3 g, about 800 mg to about 4 g, about 900 mg to about 5 g, about 1 g to about 3 g, about 1.1 g to about 6 g, about 1.2 g to about 8 g, about 1.4 g to about 10 g, about 2.5 g to about 7.5 g, about 3 g to about 12 g, about 4 g to about 15 g, about 4.5 g to about 16 g, about 5 g to about 20 g, about 6 g to about 18 g, about 7 g to about 21 g, about 8 g to about 23 g, about 9 g to about 25 g, about 10 g to about 30 g, about 12 g to about 35 g, about 14 g to about 28 g, about 15 g to about 45 g, about 18 g to about 36 g, about 20 g to about 50 g, about 25 g to about 65 g, about 30 g to about 70 g, about 35 g to about 80 g, about 40 g to about 100 g, about 45 g to about 115 g, about 50 g to about 135 g, about 20 g to about 300 g, about 20 g to about 500 g, about 25 g to about 800 g, about 70 g to about 385 g, about 75 g to about 450 g about 85 g to about 600 g, about 100 g to about 800 g, about 200 g to about 1 kg, or about 475 g to about 1 kg. In some embodiments an effective amount of the N-glycosylation inhibitor is between about 20 mg to about 60 mg. In some embodiments an effective amount of the N-glycosylation inhibitor is between about 1 g to about 40 g. In some embodiments an effective amount of the N-glycosylation inhibitor is between about 1 g to about 25 g. In some embodiments an effective amount of the N-glycosylation inhibitor is between about 1 g to about 15 g. In some embodiments an effective amount of the N-glycosylation inhibitor is between about 3 g to about 30 g. In some embodiments an effective amount of the N-glycosylation inhibitor is between about 3 g to about 20 g. In some embodiments an effective amount of the N-glycosylation inhibitor is between about 5 g to about 25 g. In some embodiments an effective amount of the N-glycosylation inhibitor is between about 5 g to about 15 g. In some embodiments an effective amount of the N-glycosylation inhibitor is between about 5 g to about 9 g. In some embodiments an effective amount of the N-glycosylation inhibitor is between about 10 g to about 40 g. In some embodiments an effective amount of the N-glycosylation inhibitor is between about 10 g to about 25 g. In some embodiments an effective amount of the N-glycosylation inhibitor is between about 10 g to about 15 g. In some embodiments, an effective amount of the N-glycosylation inhibitor administered to the subject for use in a method to treat stroke in a single treatment regimen is listed in this paragraph. In some embodiments, an effective amount of the N-glycosylation inhibitor administered to the subject for use in a method to attenuate one or more stroke symptoms in a single treatment regimen is listed in this paragraph. In some embodiments, an effective amount of the N-glycosylation inhibitor administered to the subject for use in a method to prevent stroke in a single treatment regimen is listed in this paragraph.

In some embodiments described herein, methods of multiple dose treatment regimens comprise administering a therapeutically effective about of N-glycosylation inhibitor. In some embodiments, an effective amount of an N-glycosylation inhibitor is at least about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, 1 g, 1.5 g, 2 g, 2.5 g, 3 g, 3.5 g, 4 g, 4.5 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 32 g, 34 g, 36 g, 38 g, 40 g, 42 g, 44 g, 46 g, 48 g, 50 g, 53 g, 56 g, 60 g, 63 g, 66 g, 70 g, 73 g, 76 g, 80 g, 85 g, 90 g, 95 g, 100 g, 105 g, 110 g, 115 g, 120 g, 125 g, 130 g, 135 g, 140 g, 145 g, 150 g, 155 g, 160 g, 165 g, 170 g, 175 g, 180 g, 185 g, 190 g, 195 g, 200 g, 215 g, 230 g, 245 g, 260 g, 275 g, 300 g, 325 g, 350 g, 375 g, 400 g, 430 g, 460 g, 500 g, 530 g, 560 g, 600 g, 630 g, 660 g, 700 g, 750 g, 800 g, 850 g, 900 g, 950 g, or 1 kg. In some embodiments, an effective amount of the N-glycosylation inhibitor is at least about 40 mg. In some embodiments, an effective amount of the N-glycosylation inhibitor is at least about 1 g. In some embodiments, an effective amount of the N-glycosylation inhibitor is at least about 2 g. In some embodiments, an effective amount of the N-glycosylation inhibitor is at least about 3 g. In some embodiments, an effective amount of the N-glycosylation inhibitor is at least about 4 g. In some embodiments, an effective amount of the N-glycosylation inhibitor is at least about 5 g. In some embodiments, an effective amount of the N-glycosylation inhibitor is at least about 6 g. In some embodiments, an effective amount of the N-glycosylation inhibitor is at least about 7 g. In some embodiments, an effective amount of the N-glycosylation inhibitor is at least about 8 g. In some embodiments, an effective amount of the N-glycosylation inhibitor is at least about 9 g. In some embodiments, an effective amount of the N-glycosylation inhibitor is at least about 10 g. In some embodiments, an effective amount of the N-glycosylation inhibitor is at least about 11 g. In some embodiments, an effective amount of the N-glycosylation inhibitor is at least about 12 g. In some embodiments, an effective amount of the N-glycosylation inhibitor is at least about 13 g. In some embodiments, an effective amount of the N-glycosylation inhibitor is at least about 14 g. In some embodiments, an effective amount of the N-glycosylation inhibitor is at least about 15 g. In some embodiments, an effective amount of the N-glycosylation inhibitor is at least about 18 g. In some embodiments, an effective amount of the N-glycosylation inhibitor is at least about 20 g. In some embodiments, an effective amount of the N-glycosylation inhibitor is at least about 22 g. In some embodiments, an effective amount of the N-glycosylation inhibitor is at least about 25 g. In some embodiments, an effective amount of the N-glycosylation inhibitor is at least about 27 g. In some embodiments, an effective amount of the N-glycosylation inhibitor is at least about 30 g. In some embodiments, an effective amount of the N-glycosylation inhibitor is at least about 35 g. In some embodiments, an effective amount of the N-glycosylation inhibitor is at least about 40 g. In some embodiments, an effective amount of an N-glycosylation inhibitor is no more than about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 450 mg, 500 mg, 550 mg, 600 mg, 650 mg, 700 mg, 750 mg, 800 mg, 850 mg, 900 mg, 950 mg, 1 g, 1.5 g, 2 g, 2.5 g, 3 g, 3.5 g, 4 g, 4.5 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 32 g, 34 g, 36 g, 38 g, 40 g, 42 g, 44 g, 46 g, 48 g, 50 g, 53 g, 56 g, 60 g, 63 g, 66 g, 70 g, 73 g, 76 g, 80 g, 85 g, 90 g, 95 g, 100 g, 105 g, 110 g, 115 g, 120 g, 125 g, 130 g, 135 g, 140 g, 145 g, 150 g, 155 g, 160 g, 165 g, 170 g, 175 g, 180 g, 185 g, 190 g, 195 g, 200 g, 215 g, 230 g, 245 g, 260 g, 275 g, 300 g, 325 g, 350 g, 375 g, 400 g, 430 g, 460 g, 500 g, 530 g, 560 g, 600 g, 630 g, 660 g, 700 g, 750 g, 800 g, 850 g, 900 g, 950 g, or 1 kg. In some embodiments, an effective amount of the N-glycosylation inhibitor is no more than about 40 g. In some embodiments, an effective amount of the N-glycosylation inhibitor is no more than about 35 g. In some embodiments, an effective amount of the N-glycosylation inhibitor is no more than about 30 g. In some embodiments, an effective amount of the N-glycosylation inhibitor is no more than about 25 g. In some embodiments, an effective amount of the N-glycosylation inhibitor is no more than about 20 g. In some embodiments, an effective amount of the N-glycosylation inhibitor is no more than about 18 g. In some embodiments, an effective amount of the N-glycosylation inhibitor is no more than about 15 g. In some embodiments, an effective amount of the N-glycosylation inhibitor is no more than about 12 g. In some embodiments, an effective amount of the N-glycosylation inhibitor is no more than about 10 g. In some embodiments, an effective amount of the N-glycosylation inhibitor is no more than about 8 g. In some embodiments, an effective amount of the N-glycosylation inhibitor is no more than about 6 g. In some embodiments, an effective amount of the N-glycosylation inhibitor is no more than about 4 g. In some embodiments, an effective amount of the N-glycosylation inhibitor is no more than about 2 g. In some embodiments, an effective amount of the N-glycosylation inhibitor is no more than about 1 g. In some embodiments, an effective amount of the N-glycosylation inhibitor is no more than about 40 mg. In some embodiments an effective amount of an N-glycosylation inhibitor is between about 1 mg to about 10 mg, about 3 mg to about 10 mg, about 5 mg to about 15 mg, about 10 mg to about 20 mg, about 15 mg to about 45 mg, about 20 mg to about 55 mg, about 25 mg to about 70 mg, about 35 mg to about 75 mg, about 40 mg to about 105 mg, about 45 mg to about 85 mg, about 50 mg to about 80 mg, about 55 about 125 mg, about 60 mg to about 150 mg, about 65 mg to about 185 mg, about 70 mg to about 125 mg, about 75 mg to about 200 mg, about 80 mg to about 225 mg, about 85 mg to about 300 mg, about 90 mg to about 180 mg, about 100 mg to about 150 mg, about 100 mg to about 225 mg, about 100 mg to about 350 mg, about 100 mg to about 500 mg, about 110 mg to about 200 mg, about 110 mg to about 275 mg, about 150 mg to about 375 mg, about 175 mg to about 500 mg, about 190 mg to about 315 mg, about 225 mg to about 600 mg, about 250 mg to about 700 mg, about 250 mg to about 500 mg, about 260 mg to about 800 mg, about 275 mg to about 600 mg, about 300 mg to about 900 mg, about 350 mg to about 750 mg, about 400 mg to about 2 g, about 425 mg to about 1.1 g, about 450 mg to about 1.5 g, about 500 mg to about 850 mg, about 525 mg to about 1.3 g, about 600 mg to about 2 g, about 700 mg to about 3 g, about 800 mg to about 4 g, about 900 mg to about 5 g, about 1 g to about 3 g, about 1.1 g to about 6 g, about 1.2 g to about 8 g, about 1.4 g to about 10 g, about 2.5 g to about 7.5 g, about 3 g to about 12 g, about 4 g to about 15 g, about 4.5 g to about 16 g, about 5 g to about 20 g, about 6 g to about 18 g, about 7 g to about 21 g, about 8 g to about 23 g, about 9 g to about 25 g, about 10 g to about 30 g, about 12 g to about 35 g, about 14 g to about 28 g, about 15 g to about 45 g, about 18 g to about 36 g, about 20 g to about 50 g, about 25 g to about 65 g, about 30 g to about 70 g, about 35 g to about 80 g, about 40 g to about 100 g, about 45 g to about 115 g, about 50 g to about 135 g, about 20 g to about 300 g, about 20 g to about 500 g, about 25 g to about 800 g, about 70 g to about 385 g, about 75 g to about 450 g about 85 g to about 600 g, about 100 g to about 800 g, about 200 g to about 1 kg, or about 475 g to about 1 kg. In some embodiments an effective amount of the N-glycosylation inhibitor is between about 20 mg to about 60 mg. In some embodiments an effective amount of the N-glycosylation inhibitor is between about 1 g to about 40 g. In some embodiments an effective amount of the N-glycosylation inhibitor is between about 1 g to about 25 g. In some embodiments an effective amount of the N-glycosylation inhibitor is between about 1 g to about 15 g. In some embodiments an effective amount of the N-glycosylation inhibitor is between about 3 g to about 30 g. In some embodiments an effective amount of the N-glycosylation inhibitor is between about 3 g to about 20 g. In some embodiments an effective amount of the N-glycosylation inhibitor is between about 5 g to about 25 g. In some embodiments an effective amount of the N-glycosylation inhibitor is between about 5 g to about 15 g. In some embodiments an effective amount of the N-glycosylation inhibitor is between about 5 g to about 9 g. In some embodiments an effective amount of the N-glycosylation inhibitor is between about 10 g to about 40 g. In some embodiments an effective amount of the N-glycosylation inhibitor is between about 10 g to about 25 g. In some embodiments an effective amount of the N-glycosylation inhibitor is between about 10 g to about 15 g. In some embodiments, an effective amount of the N-glycosylation inhibitor administered to the subject for use in a method to treat stroke in a multiple treatment regimen is listed in this paragraph. In some embodiments, an effective amount of the N-glycosylation inhibitor administered to the subject for use in a method to attenuate one or more stroke symptoms in a multiple treatment regimen is listed in this paragraph. In some embodiments, an effective amount of the N-glycosylation inhibitor administered to the subject for use in a method to prevent stroke in a multiple treatment regimen is listed in this paragraph.

In some embodiments described herein are methods comprising administering a formulation of the N-glycosylation inhibitor at a particular dosage per body weight of a subject. In some embodiments, the formulation comprises a dosage of N-glycosylation inhibitor of about 10 mg to about 100 mg/kg body weight of a subject. In some embodiments, the formulation comprises a dosage of N-glycosylation inhibitor of about 20 mg to about 200 mg/kg body weight of a subject. In some embodiments, the formulation comprises a dosage of N-glycosylation inhibitor of about 25 mg to about 250 mg/kg body weight of a subject. In some embodiments, the formulation comprises a dosage of N-glycosylation inhibitor of about 35 mg to about 300 mg/kg body weight of a subject. In some embodiments, the formulation comprises a dosage of N-glycosylation inhibitor of about 20 mg to about 400 mg/kg body weight of a subject. In some embodiments, the formulation comprises a dosage of N-glycosylation inhibitor of about 40 mg to about 500/kg body weight of a subject. In some embodiments, the formulation comprises a dosage of N-glycosylation inhibitor of about 50 mg to about 200 mg/kg body weight of a subject. In some embodiments, the formulation comprises a dosage of N-glycosylation inhibitor of about 55 mg to about 600 mg/kg body weight of a subject. In some embodiments, the formulation comprises a dosage of N-glycosylation inhibitor of about 85 mg to about 600 mg/kg body weight of a subject. In some embodiments, the formulation comprises a dosage of N-glycosylation inhibitor of about 125 mg to about 600 mg/kg body weight of a subject. In some embodiments, the formulation comprises a dosage of N-glycosylation inhibitor of about 215 mg to about 600 mg/kg body weight of a subject. In some embodiments, the formulation comprises a dosage of N-glycosylation inhibitor of about 325 mg to about 600 mg/kg body weight of a subject. In some embodiments, the formulation comprises a dosage of N-glycosylation inhibitor of about 425 mg to about 600 mg/kg body weight of a subject. In some embodiments, the formulation comprises a dosage of N-glycosylation inhibitor of about 250 mg to about 700 mg/kg body weight of a subject. In some embodiments, the formulation comprises a dosage of N-glycosylation inhibitor of about 315 mg to about 750 mg/kg body weight of a subject. In some embodiments, the formulation comprises a dosage of N-glycosylation inhibitor of about 355 mg to about 800 mg/kg body weight of a subject. In some embodiments, the formulation comprises a dosage of N-glycosylation inhibitor of about 435 mg to about 620 mg/kg body weight of a subject. In some embodiments, the formulation comprises a dosage of N-glycosylation inhibitor of about 1.5 mg to about 2.0 mg/gram body weight of a subject. In some embodiments, the formulation comprises a dosage of N-glycosylation inhibitor of about 50 mg to about 600 mg/kg body weight of a subject. In some embodiments, the formulation comprises a dosage of N-glycosylation inhibitor of about 75 mg to about 300 mg/kg body weight of a subject. In some embodiments, the formulation comprises a dosage of N-glycosylation inhibitor of about 100 mg to about 200 mg/kg body weight of a subject. In some embodiments, the formulation comprises a dosage of N-glycosylation inhibitor of about 130 mg to about 163 mg/kg body weight of a subject. In some embodiments, the formulation comprises a dosage of N-glycosylation inhibitor of about 130 mg to about 300 mg/kg body weight of a subject. In some embodiments, the formulation comprises a dosage of N-glycosylation inhibitor of about 130 mg to about 660 mg/kg body weight of a subject. Described herein are methods of preventing, attenuating, or treating stroke in a subject comprising administering a pharmaceutical composition described herein at a particular dose of the one or more N-glycosylation inhibitors in a single treatment regimen or a multiple treatment regimen. In some embodiments, the dose administered to the subject is determined by a dose selection. In some embodiments, a therapeutically effective dose is determined through experimental evaluation using an animal. In some embodiments, the animal is a mouse, a rat, a rabbit, a guinea pig, a cat, a dog, or a pig. In some embodiments, the dose selection is determined by calculating a human equivalent dose (HED). In some embodiments, the dose administered to the subject is a human equivalent dose (HED). In some embodiments, the HED is calculated according to a dose by factor method. In some embodiments, the HED is calculated based on body surface area comparison between species. In some embodiments, the HED is calculated based on body surface area comparison between mouse and human. In some embodiments, a HED is calculated based on body surface area comparison between mouse and human in which to animal equivalent dose (AED) in units of mg/kg body weight to a HED) in units of mg/kg body weight, the AED is divided by a factor of 12.3. In some embodiments, the dose by factor method uses the no observed adverse effect levels (NOAEL) of drug from a preclinical toxicological study to estimate HED. In some embodiments, the dose selection is based on minimum risk of toxicity in humans. In some embodiments, the dose selection is based on a minimum pharmacologic activity in humans. In some embodiments, the dose selection is based on existing pharmacokinetics data for another drug of the same pharmacological category. In some embodiments, the another drug of the same pharmacological category is a hexosamine. In some embodiments, the hexosamine is a mannosamine. In some embodiments, the hexosamine is a fructosamine. In some embodiments, the hexosamine is a galactosamine. In some embodiments, the hexosamine is a glucosamine. In some embodiments, the mannosamine is N-Acetyl-D-Mannosamine (ManNAc), diazirine-derivatized mannosamine (ManNDAz), tetraacetylated N-azidoacetyl-d-mannosamine (ManNAcAz), N-glycolyl-D-mannosamine, 2-N-acetyl-6-O-acetyl-D-mannosamine, N-Levulinoyl mannosamine (Man2NLev), N-azido-acetylmannosamine (Man2NAz), or L-mannosamine. In some embodiments, the hexosamine is listed in Table1. In some embodiments, the another drug of the same pharmacological category is listed in Table 1. In some embodiments, the dose administered to the subject is determined based on drug activity instead of scaling of dose among species. In some embodiments, the dose administered to the subject is determined by following one or more guidelines described in Nair A B, Jacob S. A simple practice guide for dose conversion between animals and human. J Basic Clin Pharm. 2016 March; 7 (2): 27-31, the contents of which are herein incorporated by reference.

### Stroke Prevention

Described herein are methods to prevent stroke comprising administering to a subject in need thereof an effective amount of one or more N-glycosylation inhibitor. In some embodiments, the one or more N-glycosylation inhibitors comprise hexosamine D-mannosamine (ManN), Kifunensine (Kif), Castanospermine (Cas), or a combination thereof. In some embodiments, the one or more N-glycosylation inhibitors comprise ManN and Kif. In some embodiments, the one or more N-glycosylation inhibitors comprise ManN and Cas. In some embodiments, the one or more N-glycosylation inhibitors comprise Kif and Cas. In some embodiments, the one or more N-glycosylation inhibitors comprise ManN, Kif, and Cas. In some embodiments, the N-glycosylation inhibitor comprises ManN. In some embodiments, the N-glycosylation inhibitor comprises Kif. In some embodiments, the N-glycosylation inhibitor comprises Cas. In some embodiments, the N-glycosylation inhibitor consists essentially of ManN. In some embodiments, the one or more N-glycosylation inhibitors is listed in Table 1.

In some embodiments, the subject in need of administration of a N-glycosylation inhibitor is a mammal. In some embodiments, the subject is a rodent. In some embodiments, the subject belongs to the genus Rattus. In some embodiments, the subject belongs to the genus Mus. In some embodiments, the subject belongs to the genus Canis. In some embodiments, the subject belongs to the genus Felis. In some embodiments, the subject belongs to the genus Equus. In some embodiments, the subject is a human.

In some embodiments described herein, methods to prevent stroke comprise a single dose treatment regimen. In some embodiments described herein, methods to prevent stroke comprise a multiple dose treatment regimen.

In some embodiments described herein, methods to prevent stroke comprise a reduced risk of recurrent stroke in a subject. In some embodiments, a subject has previously experienced an ischemic stroke, a hemorrhagic stroke, a brainstem stroke, a transient ischemic attack, or a cryptogenic stroke. In some embodiments, the risk of a recurrent stroke comprises a risk of an ischemic stroke, a hemorrhagic stroke, a brainstem stroke, a transient ischemic attack, or a cryptogenic stroke.

### Stroke Risk Markers

In some embodiments described herein, methods to prevent stroke comprise a measurement of C-reactive protein (CRP) in the blood in a subject. In some embodiments, a measurement of C-reactive protein (CRP) in the blood in a subject is reduced compared to a previous measurement of blood CRP level in the subject. In some embodiments, the measurement of CRP in the blood in a subject is reduced to below about 10 mg/L.

### Symptoms of Stroke

In some embodiments, are methods of prevention, attenuation, or treatment of stroke in a subject in need thereof wherein the subject is selected for administration of a pharmaceutical composition described herein. In some embodiments, the subject is selected due to having previously had one or more TIAs. In some embodiments, the subject is selected to begin treatment following a diagnosis of having experienced one or more TIAs. In some embodiments, a subject experiencing one or more symptoms commonly found during a TIA is used to diagnose the subject as having had one or more TIAs. In some embodiments, the one or more symptoms commonly found during a TIA comprise muscle weakness, numbness in the face, paralysis in the face, paralysis in a limb, paralysis on one side of the body, slurred speech, garbled speech, difficulty understanding others, sudden onset blindness in one or both eyes, double vision, vertigo, confusion, lack of mental alertness, loss of balance, or loss of coordination, or any combination thereof. In some embodiments, the subject is selected following observation of signs of carotid artery disease in the subject, which is a common cause of ischemic stroke. In some embodiments, the subject is selected following diagnosis of a stroke. In some embodiments, computed tomography (CT) scanning is used to identify hemorrhage in the brain indicating diagnosis of a stroke. In some embodiments, CT scanning is used to identify damage in the brain indicating diagnosis of a stroke. In some embodiments, magnetic resonance imaging (MRI) scanning is used to identify damage in the brain indicating diagnosis of a stroke. In some embodiments, imaging tests revealing narrowed blood vessels in the neck, an aneurysm, or tangled blood vessels in the brain are used for diagnosis of a stroke. In some embodiments, a subject experiencing one or more symptoms commonly found during a stroke is select the subject for a method of treatment described herein. In some embodiments, the one or more symptoms commonly found during a stroke comprise muscle weakness, numbness in the face, paralysis in the face, paralysis in a limb, paralysis on one side of the body, slurred speech, garbled speech, difficulty understanding others, sudden onset blindness in one or both eyes, double vision, vertigo, confusion, lack of mental alertness, loss of balance, or loss of coordination, or any combination thereof. In some embodiments, the method comprises a reduction in mortality in the subject following the administering. In some embodiments, the method comprises a reduction in mortality in the subject compared to administration of an anticoagulant as a monotherapy, a tissue plasminogen activator (TPA) as a monotherapy, an antiplatelet medication as a monotherapy, or an antihypertensive medication as a monotherapy in a control subject. In some embodiments, the method comprises a reduction in mortality in the subject compared to administration of an anticoagulant, a tissue plasminogen activator, an antiplatelet medication, or an antihypertensive medication, or any combination thereof in a control subject. In some embodiments, the method comprises a reduction in mortality in the subject compared to a control subject that receives either no therapy or a vehicle control therapy. In some embodiments, the method comprises an improvement in one or more symptoms of stroke in the subject following the administering. In some embodiments, the method comprises a reduction in an area or size of damaged neural tissue in the brain of the subject following the administering. In some embodiments, the method comprises a rescue of hypoxic damage to one or more areas of the brain of the subject following the administering. In some embodiments, the method comprises a reversing of stroke-induced brain injury in the subject following the administering. In some embodiments, the method comprises a reversing of stroke-induced memory impairment in the subject following the administering. In some embodiments, the method comprises a reversing of stroke-induced voluntary movement impairment in the subject following the administering. In some embodiments, the method comprises a reversing of stroke-induced language impairment in the subject following the administering. In some embodiments, the method comprises an improvement in cognitive capacity in the subject following the administering. In some embodiments, the method comprises an improvement in learning in the subject following the administering. In some embodiments, the method comprises an improvement in memory in the subject following the administering. In some embodiments, the method comprises an improvement in verbal or non-verbal communication in the subject following the administering. In some embodiments, the method comprises an improvement in mobility in the subject following the administering. In some embodiments, the method comprises a reversal of stroke-induced brain injury in the subject following the administering. In some embodiments, the method comprises a reversal of stroke-induced cognitive and/or memory impairment in the subject following the administering. In some embodiments, the method comprises a reversal of stroke-induced voluntary muscle control impairment in the subject following the administering. In some embodiments, the method comprises a reversal of infarct induced brain damage in the subject following the administering. In some embodiments, the method comprises a reduction in hemorrhagic transformation of an ischemic event in the subject following the administering. In some embodiments, the reduction in hemorrhagic transformation is determined by measuring the subject's hemoglobin level compared to administration of an anticoagulant alone as a monotherapy.

### Attenuating an Ongoing Ischemic Event

Described herein are methods to attenuate an ongoing ischemic event affecting the brain. In some embodiments, the methods comprise administering to a subject in need thereof an effective amount of an N-glycosylation inhibitor concurrently with an ischemic event. In some embodiments, the ischemic comprising a subject experiencing an ischemic stroke, a hemorrhagic stroke, a brainstem stroke, a transient ischemic attack, or a cryptogenic stroke. In some embodiments described herein, an effective amount of an N-glycosylation inhibitor is administered orally, intravenously, intrathecally, and/or intraperitoneally.

### Neurological Assessment and Blood Flow Restoration

Described herein are methods to attenuate an ongoing ischemic event affecting the brain. In some embodiments, a neurological assessment is given to a subject during an ischemic event affecting the brain. In some embodiments, a neurological assessment is given to a subject after an ischemic event has affected the brain. In some embodiments, multiple neurological assessments are given to a subject during an ischemic event affecting the brain. In some embodiments, multiple neurological assessments are given to a subject after an ischemic event has affected the brain. In some embodiments, the neurological assessments taken at different times are compared. In some embodiments described herein, a neurological assessment of acute stroke in a subject improves compared to a previous assessment. In some embodiments described herein, blood flow to the brain of a subject is improved. In some embodiments described herein, the blood flow to the brain is assessed by transcranial doppler ultrasound.

Compounds with Chemical Structure Similar to ManN

In some embodiments of methods of prevention, attenuation, or treatment of stroke described herein, one or more compounds comprising a chemical structural similar to ManN are administered to the subject in need thereof. In some embodiments, the one or more compounds comprising a chemical structural similar to ManN are administered in combination with ManN. In some embodiments, the one or more compounds comprising a chemical structural similar to ManN are administered in combination with Kif. In some embodiments, the one or more compounds comprising a chemical structural similar to ManN are administered in combination with Cas. In some embodiments, the one or more compounds comprising a chemical structural similar to ManN administered to the subject function as N-glycosylation inhibitors. In some embodiments, the one or more compounds comprising a chemical structural similar to ManN are listed in Table 1. In some embodiments, a compound listed in table one is administered to a subject in a method of prevention, attenuation, or treatment of stroke described herein.

TABLE 1
Compounds with chemical structure similar to ManN

| PubChem CID | Cmpd Name | IUPAC Name |
|---|---|---|
| 1167 | Uridine diphosphate-N- | [3-acetamido-4,5-dihydroxy-6- |
| | acetylgalactosamine | (hydroxymethyl)oxan-2-yl] [[5-(2,4- |
| | | dioxopyrimidin-1-yl)-3,4- |
| | | dihydroxyoxolan-2-yl]methoxy- |
| | | hydroxyphosphoryl] hydrogen phosphate |
| 65150 | N-((2S,3R,4S,5R)-3,4,5,6- | N-[(2S,3R,4S,5R)-3,4,5,6-tetrahydroxy- |
| | Tetrahydroxy-1-oxohexan-2- | 1-oxohexan-2-yl]acetamide |
| | yl)acetamide | |
| 79642 | Mannosamine HCl | (2S,3R,4S,5R)-2-amino-3,4,5,6- |
| | | tetrahydroxyhexanoic acid; hydrochloride |
| 123961 | 2-Amino-2-deoxymannose | (2S,3R,4S,5R)-2-amino-3,4,5,6- |
| | | tetrahydroxyhexanal |
| 134218 | Methyl 6-amino-6-deoxy-beta- | (2R,3R,4S,5R,6R)-2-(aminomethyl)-6- |
| | D-Galactopyranoside | methoxyoxane-3,4,5-triol |
| 161531 | [(3S,4R,5S,6R)-3-acetamido- | [(3S,4R,5S,6R)-3-acetamido-4,5- |
| | 4,5-dihydroxy-6- | dihydroxy-6-(hydroxymethyl)oxan-2-yl] |
| | (hydroxymethyl)oxan-2-yl] | [[(2R,3S,4R,5R)-5-(2,4-dioxopyrimidin- |
| | [[(2R,3S,4R,5R)-5-(2,4- | 1-yl)-3,4-dihydroxyoxolan-2-yl]methoxy- |
| | dioxopyrimidin-1-yl)-3,4- | hydroxyphosphoryl] hydrogen phosphate |
| | dihydroxyoxolan-2-yl]methoxy- | |
| | hydroxyphosphoryl] hydrogen | |
| | phosphate | |
| 177150 | alpha-D-Mannopyranoside, | (2S,3S,4S,5S,6R)-5-amino-2-methoxy-6- |
| | methyl 4-amino-4,6-dideoxy- | methyloxane-3,4-diol |
| 191160 | Methyl-2-amino-2-deoxy-alpha- | (2R,3S,4R,5S,6S)-5-amino-2- |
| | D-mannopyranoside | (hydroxymethyl)-6-methoxyoxane-3,4- |
| | | diol |
| 191160 | Methyl-2-amino-2-deoxy-alpha- | (2R,3S,4R,5S,6S)-5-amino-2- |
| | D-mannopyranoside | (hydroxymethyl)-6-methoxyoxane-3,4- |
| | | diol |
| 318324 | 2-Amino-3,4,5,6- | 2-amino-3,4,5,6-tetrahydroxyhexanal |
| | tetrahydroxyhexanal | |
| 350355 | Methyl 2-amino-2-deoxy-a-D- | 5-amino-2-(hydroxymethyl)-6- |
| | mannopyranoside | methoxyoxane-3,4-diol |
| 419561 | 4-Bromodiphenylsulfide | 2-(aminomethyl)-6-methoxyoxane-3,4,5- |
| | | triol |
| 428542 | 2.6-Didesoxy-2-aminohexose | 3-amino-6-methyloxane-2,4,5-triol |
| 429010 | 3-Amino-6-(hydroxymethyl)-4- | 3-amino-6-(hydroxymethyl)-4- |
| | methoxyoxane-2,5-diol | methoxyoxane-2,5-diol |
| 430566 | 3-Amino-6-(hydroxymethyl)- | 3-amino-6-(hydroxymethyl)-4,5- |
| | 4,5-dimethoxyoxan-2-ol | dimethoxyoxan-2-ol |
| 436413 | 3-Aminooxane-2,4,5-triol | 3-aminooxane-2,4,5-triol |
| 439281 | N-Acetyl-D-Mannosamine | N-[(3S,4R,5S,6R)-2,4,5-trihydroxy-6- |
| | | (hydroxymethyl)oxan-3-yl]acetamide |
| 440036 | Glycolyl-D-mannosamine | 2-hydroxy-N-[(2S,3S,4R,5S,6R)-2,4,5- |
| | | trihydroxy-6-(hydroxymethyl)oxan-3- |
| | | yl]acetamide |
| 440273 | 2-(acetylamino)-2-deoxy-6-O- | [(2R,3S,4R,5S,6S)-5-acetamido-3,4,6- |
| | phosphono-alpha-D- | trihydroxyoxan-2-yl]methyl dihydrogen |
| | mannopyranose | phosphate |
| 440300 | N-Glycolyl-D-mannosamine 6- | [(2R,3S,4R,5S,6R)-3,4,6-trihydroxy-5- |
| | phosphate | [(2-hydroxyacetyl)amino]oxan-2- |
| | | yl]methyl dihydrogen phosphate |
| 444139 | 4,6-Dideoxy-4-amino-alpha-D- | (2S,3R,4S,5S,6R)-5-amino-6- |
| | glucose | methyloxane-2,3,4-triol |
| 447845 | 4,6-Dideoxy-4-amino-beta-D- | (2R,3R,4S,5S,6R)-5-amino-6- |
| | glucopyranoside | methyloxane-2,3,4-triol |
| 448098 | (2R,3R,4S,6S)-3-amino-6- | (2R,3R,4S,6S)-3-amino-6- |
| | (hydroxymethyl)oxane-2,4-diol | (hydroxymethyl)oxane-2,4-diol |
| 448761 | 2,6-diamino-2,3,6-trideoxy- | [(2R,3S,5R,6S)-5-azaniumyl-3,6- |
| | alpha-D-glucose(2+) | dihydroxyoxan-2-yl]methylazanium |
| 448762 | 2,6-diamino-2,3,6-trideoxy- | (2S,3R,5S,6R)-3-amino-6- |
| | alpha-D-glucose | (aminomethyl)oxane-2,5-diol |
| 449043 | UDP-N-acetyl-alpha-D- | [(2R,3S,4R,5S,6R)-3-acetamido-4,5- |
| | mannosamine | dihydroxy-6-(hydroxymethyl)oxan-2-yl] |
| | | [[(2R,3S,4R,5R)-5-(2,4-dioxopyrimidin- |
| | | 1-yl)-3,4-dihydroxyoxolan-2-yl]methoxy- |
| | | hydroxyphosphoryl] hydrogen phosphate |
| 569183 | N-Acetyl-N-benzamido-tetra-O- | [5-[acetyl(benzoyl)amino]-2,3,4- |
| | acetyl-beta-d-mannosamine | triacetyloxy-6-oxohexyl] acetate |
| 644170 | 2-(Acetylamino)-2-deoxy- | N-[(2S,3S,4R,5S,6R)-2,4,5-trihydroxy-6- |
| | alpha-D-mannopyranose | (hydroxymethyl)oxan-3-yl]acetamide |
| 656422 | 5-Aminooxane-2,3,4-triol | 5-aminooxane-2,3,4-triol |
| 2783877 | (3S,4R,6S)-2-(aminomethyl)-6- | (3S,4R,6S)-2-(aminomethyl)-6- |
| | methoxyoxane-3,4,5-triol | methoxyoxane-3,4,5-triol |
| 3081581 | FAMan | 2-fluoro-N-[(2S,3R,4S,5R)-3,4,5,6- |
| | | tetrahydroxy-1-oxohexan-2-yl]acetamide |
| 3084028 | beta-D-Mannopyranose, 2- | N-[2,4,5-trihydroxy-6- |
| | (acetylamino)-2-deoxy- | (hydroxymethyl)oxan-3- |
| | | yl]acetamide; hydrate |
| 3381445 | (5-Azaniumyl-3,6- | (5-azaniumyl-3,6-dihydroxyoxan-2- |
| | dihydroxyoxan-2- | yl)methylazanium |
| | yl)methylazanium | |
| 3381446 | 3-Amino-6- | 3-amino-6-(aminomethyl)oxane-2,5-diol |
| | (aminomethyl)oxane-2,5-diol | |
| 3952072 | (2,3-Dihydroxy-6,8- | (2,3-dihydroxy-6,8- |
| | dioxabicyclo[3.2.1]octan-4- | dioxabicyclo[3.2.1]octan-4-yl)azanium |
| | yl)azanium | |
| 3952073 | 4-Amino-6,8- | 4-amino-6,8-dioxabicyclo[3.2.1]octane- |
| | dioxabicyclo[3.2.1]octane-2,3- | 2,3-diol |
| | diol | |
| 4471897 | 4,6-Didesoxy-4-aminohexos | 5-amino-6-methyloxane-2,3,4-triol |
| 4632235 | 3-Amino-6- | 3-amino-6-(hydroxymethyl)oxane-2,5- |
| | (hydroxymethyl)oxane-2,5-diol | diol |
| 5287642 | (3R,4S,5S,6R)-5-amino-3,4- | (3R,4S,5S,6R)-5-amino-3,4-dihydroxy-6- |
| | dihydroxy-6-methyloxan-2- | methyloxan-2-olate |
| | olate | |
| 5287643 | 4-Amino-4,6-dideoxy-D- | (3R,4S,5S,6R)-5-amino-6-methyloxane- |
| | glucopyranose | 2,3,4-triol |
| 5288374 | 3-Deoxy-D-glucosamine | (2S,3R,5S,6R)-3-amino-6- |
| | | (hydroxymethyl)oxane-2,5-diol |
| 5351447 | Cosamin | 2-amino-3,4,5,6- |
| | | tetrahydroxyhexanal; hydrochloride |
| 5477306 | CID 5477306 | NULL |
| 7048681 | D-beta-Mannosamine | (2R,3S,4R,5S,6R)-3-amino-6- |
| | | (hydroxymethyl)oxane-2,4,5-triol |
| 7761437 | [(1R,2S,3R,4R,5R)-2,3- | [(1R,2S,3R,4R,5R)-2,3-dihydroxy-6,8- |
| | dihydroxy-6,8- | dioxabicyclo[3.2.1]octan-4-yl]azanium |
| | dioxabicyclo[3.2.1]octan-4- | |
| | yl]azanium | |
| 7761438 | 1,6-Anhydro-beta-d- | (1R,2S,3R,4R,5R)-4-amino-6,8- |
| | glucosamine | dioxabicyclo[3.2.1]octane-2,3-diol |
| 9812814 | (2S,3S,4S,5S)-3-aminooxane- | (2S,3S,4S,5S)-3-aminooxane-2,4,5-triol |
| | 2,4,5-triol | |
| 9899264 | (2S,3R,4S,5R)-2-Amino- | (2S,3R,4S,5R)-2-amino-3,4,5,6- |
| | 3,4,5,6-tetrahydroxyhexanal | tetrahydroxyhexanal; hydrochloride |
| | hydrochloride | |
| 9964186 | (3S,4S,5S,6S)-3-amino-6- | (3S,4S,5S,6S)-3-amino-6-methyloxane- |
| | methyloxane-2,4,5-triol | 2,4,5-triol |
| 9964480 | D-Mannosamine hydrochloride | (3S,4R,5S,6R)-3-amino-6- |
| | | (hydroxymethyl)oxane-2,4,5- |
| | | triol; hydrochloride |
| 9990241 | (2R,3R,4R,5R,6S)-5-amino-4- | (2R,3R,4R,5R,6S)-5-amino-4-fluoro-2- |
| | fluoro-2-(hydroxymethyl)-6- | (hydroxymethyl)-6-methoxyoxan-3-ol |
| | methoxyoxan-3-ol | |
| 10012218 | (3S,4S,5S,6R)-4-amino-6- | (3S,4S,5S,6R)-4-amino-6-methyloxane- |
| | methyltetrahydro-2H-pyran- | 2,3,5-triol |
| | 2,3,5-triol | |
| 10878009 | Methyl 2-amino-2-deoxy-beta- | (2R,3S,4R,5R,6R)-5-amino-2- |
| | D-glucopyranoside | (hydroxymethyl)-6-methoxyoxane-3,4- |
| | | diol |
| 11096158 | N-acetylmannosamine | N-[(2R,3S,4R,5S,6R)-2,4,5-trihydroxy-6- |
| | | (hydroxymethyl)oxan-3-yl]acetamide |
| 11287661 | methyl 2-amino-2-deoxy-alpha- | (2R,3S,4R,5R,6S)-5-amino-2- |
| | D-glucopyranoside | (hydroxymethyl)-6-methoxyoxane-3,4- |
| | | diol |
| 11426583 | N-Acetyl-D-mannosamine 6- | [(2R,3S,4R,5S)-5-acetamido-3,4,6- |
| | phosphate | trihydroxyoxan-2-yl]methyl dihydrogen |
| | | phosphate |
| 11565673 | N-acetyl-mannosamine-1- | [(3S,4R,5S,6R)-3-acetamido-4,5- |
| | phosphate | dihydroxy-6-(hydroxymethyl)oxan-2-yl] |
| | | dihydrogen phosphate |
| 11600837 | (2R,3S,4S,5R,6S)-2- | (2R,3S,4S,5R,6S)-2-(aminomethyl)-6- |
| | (aminomethyl)-6- | methoxyoxane-3,4,5-triol |
| | methoxytetrahydro-2H-pyran- | |
| | 3,4,5-triol | |
| 11637467 | Mannosamine-6-phosphate | [(2R,3S,4R,5S)-5-amino-3,4,6- |
| | | trihydroxyoxan-2-yl]methyl dihydrogen |
| | | phosphate |
| 11666091 | N-propargyloxycarbonyl-d- | prop-2-ynyl N-[(3S,4R,5S,6R)-2,4,5- |
| | mannosamine | trihydroxy-6-(hydroxymethyl)oxan-3- |
| | | yl]carbamate |
| 11744954 | D-Fucosamine | (3R,4R,5R,6R)-3-amino-6-methyloxane- |
| | | 2,4,5-triol |
| 11758791 | benzyl N-[(3S,4R,5S,6R)-2,4,5- | benzyl N-[(3S,4R,5S,6R)-2,4,5- |
| | trihydroxy-6- | trihydroxy-6-(hydroxymethyl)oxan-3- |
| | (hydroxymethyl)oxan-3- | yl]carbamate |
| | yl]carbamate | |
| 11845169 | 1-O-Benzyl-N-acetyl-alpha-D- | N-[(2S,3S,4R,5S,6R)-4,5-dihydroxy-6- |
| | mannosamine | (hydroxymethyl)-2-phenylmethoxyoxan- |
| | | 3-yl]acetamide |
| 11954185 | D-Quinovosamine | (3R,4R,5S,6R)-3-amino-6-methyloxane- |
| | | 2,4,5-triol |
| 12302999 | alpha-d-Mannosamine | (2S,3S,4R,5S,6R)-3-amino-6- |
| | | (hydroxymethyl)oxane-2,4,5-triol |
| 12313043 | (2R,3S,5S,6R)-4-amino-6- | (2R,3S,5S,6R)-4-amino-6-methyloxane- |
| | methyloxane-2,3,5-triol | 2,3,5-triol |
| 12313044 | 4-Amino-6-methyloxane-2,3,5- | 4-amino-6-methyloxane-2,3,5-triol |
| | triol | |
| 12314111 | 4-Amino-4,6-dideoxy-alpha-d- | (2S,3S,4S,5S,6R)-5-amino-6- |
| | mannopyranose | methyloxane-2,3,4-triol |
| 12314112 | 4-Amino-4,6-dideoxy-d- | (3S,4S,5S,6R)-5-amino-6-methyloxane- |
| | mannopyranose | 2,3,4-triol |
| 12314398 | (3S,4R,5S,6R)-3-amino-6- | (3S,4R,5S,6R)-3-amino-6-methyloxane- |
| | methyloxane-2,4,5-triol | 2,4,5-triol |
| 13135341 | Methyl 2-amino-2- | (2R,3S,4R,5R)-5-amino-2- |
| | deoxyglucopyranoside | (hydroxymethyl)-6-methoxyoxane-3,4- |
| | | diol |
| 13135342 | (2R,3S,4S,5R,6S)-5-amino-2- | (2R,3S,4S,5R,6S)-5-amino-2- |
| | (hydroxymethyl)-6- | (hydroxymethyl)-6-methoxyoxane-3,4- |
| | methoxyoxane-3,4-diol | diol |
| 13201632 | 2-Acetamido-2-deoxy-L- | N-[(3R,4S,5R,6S)-2,4,5-trihydroxy-6- |
| | mannopyranose | (hydroxymethyl)oxan-3-yl]acetamide |
| 13989712 | (2S,3R,4S,5S,6R)-5-amino-2- | (2S,3R,4S,5S,6R)-5-amino-2-methoxy-6- |
| | methoxy-6-methyloxane-3,4- | methyloxane-3,4-diol |
| | diol | |
| 14217292 | N,3-O,4-O,5-O,6-O- | [(2R,3S,4R,5S)-5-acetamido-2,3,4- |
| | Pentaacetyl-D-mannosamine | triacetyloxy-6-oxohexyl] acetate |
| 14559219 | D-Lividosamine | (3R,5S,6R)-3-amino-6- |
| | | (hydroxymethyl)oxane-2,5-diol |
| 14560972 | 3-Amino-5-fluoro-6- | 3-amino-5-fluoro-6- |
| | (hydroxymethyl)oxane-2,4-diol | (hydroxymethyl)oxane-2,4-diol |
| 14584427 | 5-Amino-6-(hydroxymethyl)-2- | 5-amino-6-(hydroxymethyl)-2- |
| | methoxyoxane-3,4-diol | methoxyoxane-3,4-diol |
| 14584428 | Methyl 4-amino-4-deoxy-alpha- | (2S,3R,4S,5S,6S)-5-amino-6- |
| | D-glucopyranoside | (hydroxymethyl)-2-methoxyoxane-3,4- |
| | | diol |
| 14701893 | (2R,3R,4R,5R,6S)-2- | (2R,3R,4R,5R,6S)-2-(aminomethyl)-6- |
| | (aminomethyl)-6- | methoxyoxane-3,4,5-triol |
| | methoxyoxane-3,4,5-triol | |
| 16069983 | N-glycoloyl-D-mannosamine 6- | [(2R,3S,4R,5S)-3,4,6-trihydroxy-5-[(2- |
| | phosphate | hydroxyacetyl)amino]oxan-2-yl]methyl |
| | | dihydrogen phosphate |
| 16069986 | aldehydo-N-acetyl-D- | [(2R,3S,4R,5S)-5-acetamido-2,3,4- |
| | mannosamine 6-phosphate | trihydroxy-6-oxohexyl] dihydrogen |
| | | phosphate |
| 16069987 | N-Glycolyl-D-mannosamine | 2-hydroxy-N-[(2S,3R,4S,5R)-3,4,5,6- |
| | | tetrahydroxy-1-oxohexan-2-yl]acetamide |
| 17756759 | 4-amino-4-deoxy-beta-L- | (2S,3R,4S,5S)-5-aminooxane-2,3,4-triol |
| | arabinopyranose | |
| 17756760 | 4-amino-4-deoxy-alpha-L- | (2R,3R,4S,5S)-5-aminooxane-2,3,4-triol |
| | arabinopyranose | |
| 17756761 | 4-amino-4-deoxy-L- | (3R,4S,5S)-5-aminooxane-2,3,4-triol |
| | arabinopyranose | |
| 18534141 | 5-Amino-2-(hydroxymethyl)-6- | 5-amino-2-(hydroxymethyl)-6- |
| | methoxyoxan-3-ol | methoxyoxan-3-ol |
| 18654014 | (3R,4R,5R,6R)-3-amino-6- | (3R,4R,5R,6R)-3-amino-6- |
| | (hydroxymethyl)-4-iodooxane- | (hydroxymethyl)-4-iodooxane-2,5-diol |
| | 2,5-diol | |
| 20054977 | 3-Amino-3,6-dideoxy-beta-D- | (2R,3S,4S,5S,6R)-4-amino-6- |
| | mannopyranose | methyloxane-2,3,5-triol |
| 21125650 | (3S,4R,5S,6S)-3-amino-6- | (3S,4R,5S,6S)-3-amino-6- |
| | (fluoromethyl)oxane-2,4,5-triol | (fluoromethyl)oxane-2,4,5-triol |
| 21155398 | (3S,4R,5R)-5-aminooxane- | (3S,4R,5R)-5-aminooxane-2,3,4-triol |
| | 2,3,4-triol | |
| 21589882 | 1-Allyl-1-C-methyl-N,3-O,4- | [(2R,3S,4R,5S,6R)-5-acetamido-3,4- |
| | O,6-O-tetraacetyl-1-deoxy- | diacetyloxy-6-methyl-6-prop-2-enyloxan- |
| | alpha-D-mannosamine | 2-yl]methyl acetate |
| 22660039 | (3S,4S,6S)-2-(aminomethyl)-6- | (3S,4S,6S)-2-(aminomethyl)-6- |
| | methoxyoxane-3,4,5-triol | methoxyoxane-3,4,5-triol |
| 22866547 | WURCS = 2.0/1,1,0/[a2122m- | (2R,3R,4R,5S,6R)-3-amino-6- |
| | 1b_1-5_2*N]/1 | methyloxane-2,4,5-triol |
| 22868018 | N-Acetyl-D-mannosamine | N-[(2S,3R,4S,5R)-3,4,5,6-tetrahydroxy- |
| | hydrate | 1-oxohexan-2-yl]acetamide; hydrate |
| 22887251 | (2R,3S,4S,6R)-5-amino-2- | (2R,3S,4S,6R)-5-amino-2- |
| | (hydroxymethyl)-6- | (hydroxymethyl)-6-methoxyoxane-3,4- |
| | methoxyoxane-3,4-diol | diol |
| 22952041 | aldehydo-N-acetyl-D- | N-[(2R,3S,4R,5S)-3,4,5,6-tetrahydroxy- |
| | mannosamine | 1-oxohexan-2-yl]acetamide |
| 23234147 | [(2S,3R,4R,5S,6S)-6- | [(2S,3R,4R,5S,6S)-6-(fluoromethyl)- |
| | (fluoromethyl)-2,4,5- | 2,4,5-trihydroxyoxan-3-yl]azanium |
| | trihydroxyoxan-3-yl]azanium | |
| 23234148 | (2S,3R,4R,5S,6S)-3-amino-6- | (2S,3R,4R,5S,6S)-3-amino-6- |
| | (fluoromethyl)oxane-2,4,5-triol | (fluoromethyl)oxane-2,4,5-triol |
| 23591751 | 3-(Chloroamino)-6- | 3-(chloroamino)-6- |
| | (hydroxymethyl)oxane-2,4,5- | (hydroxymethyl)oxane-2,4,5-triol |
| | triol | |
| 25203625 | [(3S,4S,5R,6S)-4,5,6- | [(3S,4S,5R,6S)-4,5,6-trihydroxyoxan-3- |
| | trihydroxyoxan-3-yl]azanium | yl]azanium |
| 25245190 | uridine diphosphate N- | [(2R,3S,4R,5S,6R)-3-acetamido-4,5- |
| | acetylmannosamine | dihydroxy-6-(hydroxymethyl)oxan-2-yl] |
| | | [[(2R,3S,4R,5R)-5-(2,4-dioxopyrimidin- |
| | | 1-yl)-3,4-dihydroxyoxolan-2-yl]methoxy- |
| | | oxidophosphoryl] phosphate |
| 25788878 | L-Mannosamine | (2R,3S,4R,5S)-2-amino-3,4,5,6- |
| | | tetrahydroxyhexanal |
| 25789452 | (2R,3S,4S,5R,6R)-2- | (2R,3S,4S,5R,6R)-2-(aminomethyl)-6- |
| | (aminomethyl)-6- | methoxyoxane-3,4,5-triol |
| | methoxyoxane-3,4,5-triol | |
| 44476351 | 6-Amino MMP | (2R,3S,4S,5S,6S)-2-(aminomethyl)-6- |
| | | methoxyoxane-3,4,5-triol |
| 45079006 | 4-Amino-4-deoxy-beta-D- | (2R,3R,4R,5R)-5-aminooxane-2,3,4-triol |
| | ribopyranose | |
| 45079014 | 2-Amino-2-deoxy-alpha-L- | (2R,3S,4S,5S)-3-aminooxane-2,4,5-triol |
| | xylopyranose | |
| 45082874 | 4-Amino-4-deoxy-alpha-D- | (2S,3R,4R,5R)-5-aminooxane-2,3,4-triol |
| | ribopyranose | |
| 45266685 | N-acetyl-D-mannosamine 6- | [(2R,3S,4R,5S)-5-acetamido-2,3,4- |
| | phosphate(2-) | trihydroxy-6-oxohexyl] phosphate |
| 46705338 | (3S,5R)-2-(aminomethyl)-6- | (3S,5R)-2-(aminomethyl)-6- |
| | methoxyoxane-3,4,5-triol | methoxyoxane-3,4,5-triol |
| 46780169 | N-Carbobenzyloxy | benzyl N-[(4R,5S)-2,4,5-trihydroxy-6- |
| | Mannosamine | (hydroxymethyl)oxan-3-yl]carbamate |
| 46878423 | UDP-N-acetyl-D- | [(3S,4R,5S,6R)-3-acetamido-4,5- |
| | mannosamine(2-) | dihydroxy-6-(hydroxymethyl)oxan-2-yl] |
| | | [[(2R,3S,4R,5R)-5-(2,4-dioxopyrimidin- |
| | | 1-yl)-3,4-dihydroxyoxolan-2-yl]methoxy- |
| | | oxidophosphoryl] phosphate |
| 46936396 | WURCS = 2.0/1,1,0/[a1211m- | (2S,3S,4R,5R,6S)-5-amino-6- |
| | 1b_1-5_4*N]/1 | methyloxane-2,3,4-triol |
| 46936460 | (2S,3S,5R,6S)-3-amino-6- | (2S,3S,5R,6S)-3-amino-6- |
| | (hydroxymethyl)oxane-2,5-diol | (hydroxymethyl)oxane-2,5-diol |
| 49864611 | 1-O,3-O,4-O,6-O-Tetraacetyl- | [(2R,3S,4R,5S,6R)-3,4,6-triacetyloxy-5- |
| | N-(acetoxyacetyl)-alpha-D- | [(2-acetyloxyacetyl)amino]oxan-2- |
| | mannosamine | yl]methyl acetate |
| 49864612 | 1-O,3-O,4-O,6-O-Tetraacetyl- | [(2R,3S,4R,5S,6R)-3,4,6-triacetyloxy-5- |
| | N-[(acetylthio)acetyl]-alpha-D- | [(2-acetylsulfanylacetyl)amino]oxan-2- |
| | mannosamine | yl]methyl acetate |
| 50990897 | (2R,3R,5S,6R)-3-amino-6- | (2R,3R,5S,6R)-3-amino-6- |
| | (hydroxymethyl)oxane-2,5-diol | (hydroxymethyl)oxane-2,5-diol |
| 50990898 | (2R,3R,4R,5R)-3-aminooxane- | (2R,3R,4R,5R)-3-aminooxane-2,4,5-triol |
| | 2,4,5-triol | |
| 50990899 | (2R,3R,5S,6R)-3-amino-6- | (2R,3R,5S,6R)-3-amino-6-methyloxane- |
| | methyloxane-2,5-diol | 2,5-diol |
| 52921625 | alpha-D-ManAc-(1-P-6)-alpha- | [(2R,3S,4R,5S,6R)-3-acetamido-6- |
| | D-ManAc-(1-P-6)-alpha-D- | [[[(2R,3S,4R,5S,6R)-3-acetamido-4,5- |
| | ManAc | dihydroxy-6-(hydroxymethyl)oxan-2- |
| | | yl]oxy-hydroxyphosphoryl]oxymethyl]- |
| | | 4,5-dihydroxyoxan-2-yl] |
| | | [(2R,3S,4R,5S,6S)-5-acetamido-3,4,6- |
| | | trihydroxyoxan-2-yl]methyl hydrogen |
| | | phosphate |
| 52921661 | alpha-D-ManAc-(1-P-6)-alpha- | [(2R,3S,4R,5S,6R)-3-acetamido-4,5- |
| | D-ManAc | dihydroxy-6-(hydroxymethyl)oxan-2-yl] |
| | | [(2R,3S,4R,5S,6S)-5-acetamido-3,4,6- |
| | | trihydroxyoxan-2-yl]methyl hydrogen |
| | | phosphate |
| 53481607 | UDP-N-acetyl-D-mannosamine | [(3R,4S,5R,6S)-3-acetamido-4,5- |
| | | dihydroxy-6-(hydroxymethyl)oxan-2-yl] |
| | | [[(2S,3R,4S,5S)-5-(2,4-dioxopyrimidin- |
| | | 1-yl)-3,4-dihydroxyoxolan-2-yl]methoxy- |
| | | hydroxyphosphoryl] hydrogen phosphate |
| 53486342 | (3S,5S,6R)-2-(aminomethyl)-6- | (3S,5S,6R)-2-(aminomethyl)-6- |
| | methoxyoxane-3,4,5-triol | methoxyoxane-3,4,5-triol |
| 53486344 | Methyl 6-amino-6-deoxy-beta- | (3S,5R,6R)-2-(aminomethyl)-6- |
| | D-erythro-hexopyranoside | methoxyoxane-3,4,5-triol |
| 53686052 | (2R,3R,4S,5R)-2- | (2R,3R,4S,5R)-2-(aminomethyl)-6- |
| | (aminomethyl)-6- | methoxyoxane-3,4,5-triol |
| | methoxyoxane-3,4,5-triol | |
| 53686053 | Methyl 6-amino-6-deoxy- | (2R,3S,4S,5R)-2-(aminomethyl)-6- |
| | galactopyranoside | methoxyoxane-3,4,5-triol |
| 53687325 | (3S,4R,5S,6R)-3-amino-6- | (3S,4R,5S,6R)-3-amino-6- |
| | (hydroxymethyl)-4- | (hydroxymethyl)-4-methyloxane-2,5-diol |
| | methyloxane-2,5-diol | |
| 53716703 | (2R)-3-amino-6- | (2R)-3-amino-6-(hydroxymethyl)oxane- |
| | (hydroxymethyl)oxane-2,4-diol | 2,4-diol |
| 53818929 | (3R,4R,5R,6R)-3-amino-4- | (3R,4R,5R,6R)-3-amino-4-fluoro-6- |
| | fluoro-6- | (hydroxymethyl)oxane-2,5-diol |
| | (hydroxymethyl)oxane-2,5-diol | |
| 53849650 | 3-Amino-3,6-dideoxy-alpha-L- | (2R,3R,4R,5S,6S)-4-amino-6- |
| | talopyranose | methyloxane-2,3,5-triol |
| 54152200 | (3S,4S,5R,6R)-4-amino-6- | (3S,4S,5R,6R)-4-amino-6-methyloxane- |
| | methyloxane-2,3,5-triol | 2,3,5-triol |
| 54164611 | Ethyl 2-amino-2-deoxy-beta-D- | (2R,3S,4R,5R,6R)-5-amino-6-ethoxy-2- |
| | glucopyranoside | (hydroxymethyl)oxane-3,4-diol |
| 54314445 | (3S,5S,6R)-3-amino-6- | (3S,5S,6R)-3-amino-6- |
| | (hydroxymethyl)oxane-2,5-diol | (hydroxymethyl)oxane-2,5-diol |
| 54336358 | beta-D-Fucosaminepyranose | (2R,3R,4R,5R,6R)-3-amino-6- |
| | | methyloxane-2,4,5-triol |
| 54346230 | (2R,3S,4S,5S,6R)-5-amino-2- | (2R,3S,4S,5S,6R)-5-amino-2- |
| | (hydroxymethyl)-6- | (hydroxymethyl)-6-methoxyoxane-3,4- |
| | methoxyoxane-3,4-diol | diol |
| 54346231 | Methyl 2-amino-2-deoxy-alpha- | (2R,3R,4R,5R,6S)-5-amino-2- |
| | d-galactopyranoside | (hydroxymethyl)-6-methoxyoxane-3,4- |
| | | diol |
| 54426190 | (3S,4R,5S,6R)-3-amino-6- | (3S,4R,5S,6R)-3-amino-6- |
| | (hydroxymethyl)-4- | (hydroxymethyl)-4-methoxyoxane-2,5- |
| | methoxyoxane-2,5-diol | diol |
| 54537732 | (2R,3S,4R,5R,6R)-4,5-diamino- | (2R,3S,4R,5R,6R)-4,5-diamino-2- |
| | 2-(hydroxymethyl)-6- | (hydroxymethyl)-6-methoxyoxan-3-ol |
| | methoxyoxan-3-ol | |
| 54551873 | (2S,3R,4S,5R,6R)-5-amino-2- | (2S,3R,4S,5R,6R)-5-amino-2-methoxy-6- |
| | methoxy-6-methyloxane-3,4- | methyloxane-3,4-diol |
| | diol | |
| 54551874 | Methyl 4-amino-4,6-dideoxy- | (2S,3S,4R,5R,6R)-5-amino-2-methoxy-6- |
| | alpha-D-idopyranoside | methyloxane-3,4-diol |
| 54758653 | N-acetyl-mannosamine 6- | [(2R,3S,4R,5S)-5-acetamido-3,4,6- |
| | phosphate | trihydroxyoxan-2-yl]methyl phosphate |
| 55252504 | Methyl 6-amino-6-deoxy-D- | (2R,3S,4S,5S)-2-(aminomethyl)-6- |
| | mannopyranoside | methoxyoxane-3,4,5-triol |
| 55289988 | (3S,4R,5R)-3-aminooxane- | (3S,4R,5R)-3-aminooxane-2,4,5-triol |
| | 2,4,5-triol | |
| 56845730 | N-Acetyl-D-MannosaMine | N-[(3S,4R,5S,6R)-2,4,5-trihydroxy-6- |
| | Monohydrate | (hydroxymethyl)oxan-3- |
| | | yl]acetamide; hydrate |
| 56927718 | 3-amino-3,6-dideoxy-alpha-D- | (2S,3R,4S,5R,6R)-4-amino-6- |
| | galactopyranose | methyloxane-2,3,5-triol |
| 56996207 | (2S,3S,4R,5R,6R)-4-amino-6- | (2S,3S,4R,5R,6R)-4-amino-6- |
| | methyloxane-2,3,5-triol | methyloxane-2,3,5-triol |
| 57003609 | (2S,3S,4S,5R,6R)-3-amino-4- | (2S,3S,4S,5R,6R)-3-amino-4-fluoro-6- |
| | fluoro-6- | (hydroxymethyl)oxane-2,5-diol |
| | (hydroxymethyl)oxane-2,5-diol | |
| 57149873 | (2S,3S,4S,5R,6R)-3-amino-6- | (2S,3S,4S,5R,6R)-3-amino-6- |
| | (hydroxymethyl)-4-iodooxane- | (hydroxymethyl)-4-iodooxane-2,5-diol |
| | 2,5-diol | |
| 57712819 | (3R,4R)-5-amino-2- | (3R,4R)-5-amino-2-(hydroxymethyl)-6- |
| | (hydroxymethyl)-6- | methoxyoxane-3,4-diol |
| | methoxyoxane-3,4-diol | |
| 57834585 | (3R,4R)-3-amino-6-ethyloxane- | (3R,4R)-3-amino-6-ethyloxane-2,4,5-triol |
| | 2,4,5-triol | |
| 58372544 | (3R,4S,6R)-5-amino-2- | (3R,4S,6R)-5-amino-2-(hydroxymethyl)- |
| | (hydroxymethyl)-6- | 6-methoxyoxane-3,4-diol |
| | methoxyoxane-3,4-diol | |
| 58456001 | [(4R,5R)-2,4,5-trihydroxy-6- | [(4R,5R)-2,4,5-trihydroxy-6- |
| | (hydroxymethyl)oxan-3- | (hydroxymethyl)oxan-3-yl]azanide |
| | yl]azanide | |
| 58582838 | (3R,4R,5S,6R)-3- | (3R,4R,5S,6R)-3-(chloroamino)-6- |
| | (chloroamino)-6- | (hydroxymethyl)oxane-2,4,5-triol |
| | (hydroxymethyl)oxane-2,4,5- | |
| | triol | |
| 58609215 | (3R,4R,6R)-5-amino-2- | (3R,4R,6R)-5-amino-2-(hydroxymethyl)- |
| | (hydroxymethyl)-3,6- | 3,6-dimethoxyoxan-4-ol |
| | dimethoxyoxan-4-ol | |
| 58718650 | (4R,5R)-3-(chloroamino)-6- | (4R,5R)-3-(chloroamino)-6- |
| | (hydroxymethyl)oxane-2,4,5- | (hydroxymethyl)oxane-2,4,5-triol |
| | triol | |
| 58718675 | (3R,5R)-3-(chloroamino)-6- | (3R,5R)-3-(chloroamino)-6- |
| | (hydroxymethyl)oxane-2,4,5- | (hydroxymethyl)oxane-2,4,5-triol |
| | triol | |
| 58769237 | (5R)-3-(chloroamino)-6- | (5R)-3-(chloroamino)-6- |
| | (hydroxymethyl)oxane-2,4,5- | (hydroxymethyl)oxane-2,4,5-triol |
| | triol | |
| 59109229 | (4S,5S,6R)-2-(aminomethyl)-6- | (4S,5S,6R)-2-(aminomethyl)-6- |
| | methoxyoxane-3,4,5-triol | methoxyoxane-3,4,5-triol |
| 59125085 | 4-Amino-2-methyl-6- | 4-amino-2-methyl-6-tritiooxyoxane-3,5- |
| | tritiooxyoxane-3,5-diol | diol |
| 59187615 | (3S,4R,5S,6R)-3-amino-6- | (3S,4R,5S,6R)-3-amino-6- |
| | (hydroxymethyl)-5- | (hydroxymethyl)-5-methoxyoxane-2,4- |
| | methoxyoxane-2,4-diol | diol |
| 59350339 | CID 59350339 | NULL |
| 59854309 | (3R,4S,5S)-3-aminooxane- | (3R,4S,5S)-3-aminooxane-2,4,5-triol |
| | 2,4,5-triol | |
| 59854310 | (3R,4S,5R)-3-aminooxane- | (3R,4S,5R)-3-aminooxane-2,4,5-triol |
| | 2,4,5-triol | |
| 59893576 | [2-Ethoxy-4,5-dihydroxy-6- | [2-ethoxy-4,5-dihydroxy-6- |
| | (hydroxymethyl)oxan-3- | (hydroxymethyl)oxan-3-yl]azanium |
| | yl]azanium | |
| 59893577 | 5-Amino-6-ethoxy-2- | 5-amino-6-ethoxy-2- |
| | (hydroxymethyl)oxane-3,4-diol | (hydroxymethyl)oxane-3,4-diol |
| 59894662 | (3R,6R)-5-amino-2- | (3R,6R)-5-amino-2-(hydroxymethyl)-6- |
| | (hydroxymethyl)-6- | methoxyoxane-3,4-diol |
| | methoxyoxane-3,4-diol | |
| 59920728 | (3R,4S,6R)-2-(aminomethyl)-6- | (3R,4S,6R)-2-(aminomethyl)-6- |
| | methoxyoxane-3,4,5-triol | methoxyoxane-3,4,5-triol |
| 60108451 | 5-Amino-2-(hydroxymethyl)- | 5-amino-2-(hydroxymethyl)-3,6- |
| | 3,6-dimethoxyoxan-4-ol | dimethoxyoxan-4-ol |
| 60116189 | (2R,6R)-4-amino-6- | (2R,6R)-4-amino-6-methyloxane-2,3,5- |
| | methyloxane-2,3,5-triol | triol |
| 67203049 | (2S,3R,4S,5S,6R)-5-amino-2- | (2S,3R,4S,5S,6R)-5-amino-2- |
| | (hydroxymethyl)-6- | (hydroxymethyl)-6-methoxyoxane-3,4- |
| | methoxyoxane-3,4-diol | diol |
| 67530088 | (3R,4S,5S,6S)-5-amino-6- | (3R,4S,5S,6S)-5-amino-6- |
| | (hydroxymethyl)-2- | (hydroxymethyl)-2-methoxyoxane-3,4- |
| | methoxyoxane-3,4-diol | diol |
| 67823583 | (2S,3R,4S,5S,6R)-3-amino-6- | (2S,3R,4S,5S,6R)-3-amino-6- |
| | (hydroxymethyl)-4- | (hydroxymethyl)-4-methyloxane-2,5-diol |
| | methyloxane-2,5-diol | |
| 67823594 | (3S,5R,6R)-3-amino-6- | (3S,5R,6R)-3-amino-6- |
| | (hydroxymethyl)oxane-2,5-diol | (hydroxymethyl)oxane-2,5-diol |
| 67881980 | (2S,3R,4R,5S,6R)-3-amino-6- | (2S,3R,4R,5S,6R)-3-amino-6- |
| | (hydroxymethyl)-4- | (hydroxymethyl)-4-methoxyoxane-2,5- |
| | methoxyoxane-2,5-diol | diol |
| 68047398 | (3S,4R)-5-amino-2- | (3S,4R)-5-amino-2-(hydroxymethyl)-6- |
| | (hydroxymethyl)-6- | methoxyoxane-3,4-diol |
| | methoxyoxane-3,4-diol | |
| 68062020 | (2S,3R,4R,5R,6S)-5-amino-6- | (2S,3R,4R,5R,6S)-5-amino-6-methoxy-2- |
| | methoxy-2-methyloxane-3,4- | methyloxane-3,4-diol |
| | diol | |
| 68114633 | (2S,3R,5R,6R)-5-amino-6- | (2S,3R,5R,6R)-5-amino-6-methyloxane- |
| | methyloxane-2,3-diol | 2,3-diol |
| 68250248 | (2R,5R)-6- | (2R,5R)-6-(aminomethyl)oxane-2,4,5- |
| | (aminomethyl)oxane-2,4,5-triol | triol |
| 68256069 | (3R,4R,6R)-5-amino-2- | (3R,4R,6R)-5-amino-2-(hydroxymethyl)- |
| | (hydroxymethyl)-6- | 6-methoxyoxane-3,4-diol |
| | methoxyoxane-3,4-diol | |
| 68301933 | (3S,4R,5S,6R)-3-amino-6- | (3S,4R,5S,6R)-3-amino-6-ethyloxane- |
| | ethyloxane-2,4,5-triol | 2,4,5-triol |
| 68384812 | (3S,4S,5R,6R)-3-amino-6- | (3S,4S,5R,6R)-3-amino-6- |
| | (fluoromethyl)oxane-2,4,5-triol | (fluoromethyl)oxane-2,4,5-triol |
| 68384814 | (2S,3S,4R,5S,6S)-3-amino-6- | (2S,3S,4R,5S,6S)-3-amino-6- |
| | (fluoromethyl)oxane-2,4,5-triol | (fluoromethyl)oxane-2,4,5-triol |
| 68394219 | 6-(Aminomethyl)oxane-2,4,5- | 6-(aminomethyl)oxane-2,4,5-triol |
| | triol | |
| 68760799 | o-Mannosamine | (2S,3R,4S,5S,6R)-3-amino-6- |
| | | (hydroxymethyl)oxane-2,3,4,5-tetrol |
| 69428523 | (2S,3S,4S,5S,6R)-6- | (2S,3S,4S,5S,6R)-6-(aminomethyl)-3- |
| | (aminomethyl)-3- | methoxyoxane-2,4,5-triol |
| | methoxyoxane-2,4,5-triol | |
| 69586612 | N-Acetyl-1-O,3-O,4-O,6-O- | N-[(2S,3S,4R,5S,6R)-2,4,5- |
| | tetrabenzyl-alpha-D- | tris(phenylmethoxy)-6- |
| | mannosamine | (phenylmethoxymethyl)oxan-3- |
| | | yl]acetamide |
| 69675888 | 3-Amino-6-(aminomethyl)-5- | 3-amino-6-(aminomethyl)-5- |
| | methoxyoxane-2,4-diol | methoxyoxane-2,4-diol |
| 70006305 | (2R,3S,5R,6S)-5-amino-2- | (2R,3S,5R,6S)-5-amino-2- |
| | (hydroxymethyl)-6- | (hydroxymethyl)-6-methoxyoxan-3-ol |
| | methoxyoxan-3-ol | |
| 70256843 | (2S,3R,4S,5S,6R)-6- | (2S,3R,4S,5S,6R)-6-(aminomethyl)-3- |
| | (aminomethyl)-3- | methoxyoxane-2,4,5-triol |
| | methoxyoxane-2,4,5-triol | |
| 72836880 | (2R,3S,5S)-2-(aminomethyl)-6- | (2R,3S,5S)-2-(aminomethyl)-6- |
| | methoxyoxane-3,4,5-triol | methoxyoxane-3,4,5-triol |
| 73753427 | 3-Amino-6- | 3-amino-6-(fluoromethyl)oxane-2,4,5- |
| | (fluoromethyl)oxane-2,4,5-triol | triol |
| 73821396 | [4,5-Dihydroxy-6- | [4,5-dihydroxy-6-(hydroxymethyl)-2- |
| | (hydroxymethyl)-2- | methoxyoxan-3-yl]azanium |
| | methoxyoxan-3-yl]azanium | |
| 75596335 | 2,3,6-Trideoxy-2-aminohexose | 3-amino-6-methyloxane-2,5-diol |
| 76062583 | 3-Amino-6- | 3-amino-6-(hydroxymethyl)oxane-2,4- |
| | (hydroxymethyl)oxane-2,4-diol | diol |
| 76578767 | 3-Amino-6-ethyloxane-2,4,5- | 3-amino-6-ethyloxane-2,4,5-triol |
| | triol | |
| 86600945 | N-acetyl-4-deoxy-4-fluoro-D- | N-[(3S,4R,5S,6R)-5-fluoro-2,4- |
| | mannosamine | dihydroxy-6-(hydroxymethyl)oxan-3- |
| | | yl]acetamide |
| 86624083 | N-4-Pentynoylmannosamine | N-[(3S,4R,5S,6R)-2,4,5-trihydroxy-6- |
| | | (hydroxymethyl)oxan-3-yl]pent-4- |
| | | ynamide |
| 86720678 | (3S,4S,5S,6R)-2- | (3S,4S,5S,6R)-2-(aminomethyl)-6- |
| | (aminomethyl)-6- | methoxyoxane-3,4,5-triol |
| | methoxyoxane-3,4,5-triol | |
| 87765211 | (2R,3R,4R,5S,6R)-3- | (2R,3R,4R,5S,6R)-3-(chloroamino)-6- |
| | (chloroamino)-6- | (hydroxymethyl)oxane-2,4,5-triol |
| | (hydroxymethyl)oxane-2,4,5- | |
| | triol | |
| 88897744 | (4R,5S)-3-(chloroamino)-6- | (4R,5S)-3-(chloroamino)-6- |
| | (hydroxymethyl)oxane-2,4,5- | (hydroxymethyl)oxane-2,4,5-triol |
| | triol | |
| 88911871 | (3R,5S)-3-(chloroamino)-6- | (3R,5S)-3-(chloroamino)-6- |
| | (hydroxymethyl)oxane-2,4,5- | (hydroxymethyl)oxane-2,4,5-triol |
| | triol | |
| 88946060 | (2R,5S)-3-(chloroamino)-6- | (2R,5S)-3-(chloroamino)-6- |
| | (hydroxymethyl)oxane-2,4,5- | (hydroxymethyl)oxane-2,4,5-triol |
| | triol | |
| 88992239 | (2S,5R)-3-amino-6- | (2S,5R)-3-amino-6-(fluoromethyl)oxane- |
| | (fluoromethyl)oxane-2,4,5-triol | 2,4,5-triol |
| 89152086 | (3S,4R)-3-amino-6-ethyloxane- | (3S,4R)-3-amino-6-ethyloxane-2,4,5-triol |
| | 2,4,5-triol | |
| 89196566 | (2R)-6-(aminomethyl)oxane- | (2R)-6-(aminomethyl)oxane-2,4,5-triol |
| | 2,4,5-triol | |
| 89204881 | (2R,3S,5R,6S)-2- | (2R,3S,5R,6S)-2-(aminomethyl)-6- |
| | (aminomethyl)-6- | methoxyoxane-3,4,5-triol |
| | methoxyoxane-3,4,5-triol | |
| 89242338 | (3S)-3-(chloroamino)-6- | (3S)-3-(chloroamino)-6- |
| | (hydroxymethyl)oxane-2,4,5- | (hydroxymethyl)oxane-2,4,5-triol |
| | triol | |
| 89371349 | (2S,3R,5S,6S)-3-amino-6- | (2S,3R,5S,6S)-3-amino-6- |
| | (aminomethyl)oxane-2,5-diol | (aminomethyl)oxane-2,5-diol |
| 89375538 | (2S,4S)-6-(aminomethyl)oxane- | (2S,4S)-6-(aminomethyl)oxane-2,3,4- |
| | 2,3,4-triol | triol |
| 89489540 | (3S,4S,6S)-3-amino-6- | (3S,4S,6S)-3-amino-6- |
| | (hydroxymethyl)oxane-2,4-diol | (hydroxymethyl)oxane-2,4-diol |
| 89513853 | 6-O-methylglucosamine | (3R,4R,5S,6R)-3-amino-6- |
| | | (methoxymethyl)oxane-2,4,5-triol |
| 89513868 | (3R,6S)-3-amino-4-methoxy-6- | (3R,6S)-3-amino-4-methoxy-6- |
| | (methoxymethyl)oxan-2-ol | (methoxymethyl)oxan-2-ol |
| 89529825 | (3S)-5-amino-2- | (3S)-5-amino-2-(hydroxymethyl)-6- |
| | (hydroxymethyl)-6- | methoxyoxane-3,4-diol |
| | methoxyoxane-3,4-diol | |
| 89533616 | (3R,4R,5S,6R)-3-amino-6- | (3R,4R,5S,6R)-3-amino-6- |
| | (phosphanyloxymethyl)oxane- | (phosphanyloxymethyl)oxane-2,4,5-triol |
| | 2,4,5-triol | |
| 89591175 | (3S,5S)-3-(chloroamino)-6- | (3S,5S)-3-(chloroamino)-6- |
| | (hydroxymethyl)oxane-2,4,5- | (hydroxymethyl)oxane-2,4,5-triol |
| | triol | |
| 89719151 | (4R,5R)-3-amino-6- | (4R,5R)-3-amino-6-methyloxane-2,4,5- |
| | methyloxane-2,4,5-triol | triol |
| 89787671 | (4S,5S)-5-amino-2- | (4S,5S)-5-amino-2-(hydroxymethyl)-3,6- |
| | (hydroxymethyl)-3,6- | dimethoxyoxan-4-ol |
| | dimethoxyoxan-4-ol | |
| 89847945 | (4R)-2-(aminomethyl)-6- | (4R)-2-(aminomethyl)-6-methoxyoxane- |
| | methoxyoxane-3,4,5-triol | 3,4,5-triol |
| 89857043 | (2R,4R,5S)-2-(aminomethyl)-6- | (2R,4R,5S)-2-(aminomethyl)-6- |
| | methoxyoxane-3,4,5-triol | methoxyoxane-3,4,5-triol |
| 89983499 | (2R,4R)-3-amino-6- | (2R,4R)-3-amino-6-(hydroxymethyl)-4- |
| | (hydroxymethyl)-4- | methyloxane-2,5-diol |
| | methyloxane-2,5-diol | |
| 89985851 | (4S,5R)-3-(chloroamino)-6- | (4S,5R)-3-(chloroamino)-6- |
| | (fluoromethyl)oxane-2,4,5-triol | (fluoromethyl)oxane-2,4,5-triol |
| 89985893 | (4S,5R)-3-(chloroamino)-6- | (4S,5R)-3-(chloroamino)-6- |
| | (hydroxymethyl)oxane-2,4,5- | (hydroxymethyl)oxane-2,4,5-triol |
| | triol | |
| 89985902 | (2S,4S,5R)-3-(chloroamino)-6- | (2S,4S,5R)-3-(chloroamino)-6- |
| | (hydroxymethyl)oxane-2,4,5- | (hydroxymethyl)oxane-2,4,5-triol |
| | triol | |
| 89985905 | (5R)-3-(chloroamino)-6- | (5R)-3-(chloroamino)-6- |
| | (fluoromethyl)oxane-2,4,5-triol | (fluoromethyl)oxane-2,4,5-triol |
| 90057665 | (4R)-5-amino-2- | (4R)-5-amino-2-(hydroxymethyl)-4,6- |
| | (hydroxymethyl)-4,6- | dimethoxyoxan-3-ol |
| | dimethoxyoxan-3-ol | |
| 90062884 | (2R,3S,4R,5R,6S)-3-amino-6- | (2R,3S,4R,5R,6S)-3-amino-6- |
| | methyloxane-2,4,5-triol | methyloxane-2,4,5-triol |
| 90082218 | (4S,5S)-5-amino-2- | (4S,5S)-5-amino-2-(hydroxymethyl)-6- |
| | (hydroxymethyl)-6- | methoxyoxane-3,4-diol |
| | methoxyoxane-3,4-diol | |
| 90194599 | (3R,5R)-3-amino-6- | (3R,5R)-3-amino-6-(fluoromethyl)oxane- |
| | (fluoromethyl)oxane-2,4,5-triol | 2,4,5-triol |
| 90477743 | Acetyl-D-mannosamine,N- | N-[(2S,3R,4S,5R)-3,4,5,6-tetrahydroxy- |
| | [mannosamine-6-3H] | 1-oxo-6,6-ditritiohexan-2-yl]acetamide |
| 90657791 | WURCS = 2.0/1,1,0/[a2122m- | (2S,3R,4S,5S,6R)-4-amino-6- |
| | 1a_1-5_3*N]/1 | methyloxane-2,3,5-triol |
| 90657961 | 4-amino-4,6-dideoxy-D-glucose | [(2R,3S,4S,5R)-4,5,6-trihydroxy-2- |
| | | methyloxan-3-yl]azanium |
| 91005948 | (2R,6S)-4-amino-6- | (2R,6S)-4-amino-6-methyloxane-2,3,5- |
| | methyloxane-2,3,5-triol | triol |
| 91079271 | (2R,4S,5S,6R)-2- | (2R,4S,5S,6R)-2-(aminomethyl)-6- |
| | (aminomethyl)-6- | methoxyoxane-3,4,5-triol |
| | methoxyoxane-3,4,5-triol | |
| 91091656 | (3S,4R,6R)-5-amino-2- | (3S,4R,6R)-5-amino-2-(hydroxymethyl)- |
| | (hydroxymethyl)-6- | 6-methoxyoxane-3,4-diol |
| | methoxyoxane-3,4-diol | |
| 91092504 | (2R,3R,5S)-3-(chloroamino)-6- | (2R,3R,5S)-3-(chloroamino)-6- |
| | (hydroxymethyl)oxane-2,4,5- | (hydroxymethyl)oxane-2,4,5-triol |
| | triol | |
| 91105689 | (2S,3R,5R)-5-aminooxane- | (2S,3R,5R)-5-aminooxane-2,3,4-triol |
| | 2,3,4-triol | |
| 91118134 | N-(Phenylacetyl)-D- | 2-phenyl-N-[(2S,3R,4S,5R)-3,4,5,6- |
| | mannosamine | tetrahydroxy-1-oxohexan-2-yl]acetamide |
| 91397310 | (3S,4R,6R)-5-amino-2- | (3S,4R,6R)-5-amino-2-(hydroxymethyl)- |
| | (hydroxymethyl)-3,6- | 3,6-dimethoxyoxan-4-ol |
| | dimethoxyoxan-4-ol | |
| 91463037 | (3S,4S,6R)-2-(aminomethyl)-6- | (3S,4S,6R)-2-(aminomethyl)-6- |
| | methoxyoxane-3,4,5-triol | methoxyoxane-3,4,5-triol |
| 91466751 | (2S,3S,4R,5R)-5-aminooxane- | (2S,3S,4R,5R)-5-aminooxane-2,3,4-triol |
| | 2,3,4-triol | |
| 91580441 | (2R,4R,5S)-3-(chloroamino)-6- | (2R,4R,5S)-3-(chloroamino)-6- |
| | (hydroxymethyl)oxane-2,4,5- | (hydroxymethyl)oxane-2,4,5-triol |
| | triol | |
| 91845080 | WURCS = 2.0/1,1,0/[a1122m- | (2S,3S,4S,5S,6R)-5-amino-3-methoxy-6- |
| | 1a_1-5_2*OC_4*N]/1 | methyloxane-2,4-diol |
| 91846569 | WURCS = 2.0/1,1,0/[a2122m- | (3R,4S,5S,6R)-5-amino-3-methoxy-6- |
| | 1x_1-5_2*OC_4*N]/1 | methyloxane-2,4-diol |
| 91848273 | WURCS = 2.0/1,1,0/[a2211m- | (2R,3R,4R,5R,6S)-4-amino-6- |
| | 1a_1-5_3*N]/1 | methyloxane-2,3,5-triol |
| 91849140 | WURCS = 2.0/1,1,0/[a1211m- | (2S,3S,4R,5R,6S)-5-amino-3-methoxy-6- |
| | 1b_1-5_2*OC_4*N]/1 | methyloxane-2,4-diol |
| 91851274 | 4-Amino-4,6-dideoxy-beta-D- | (2R,3S,4S,5S,6R)-5-amino-6- |
| | mannopyranose | methyloxane-2,3,4-triol |
| 91853661 | WURCS = 2.0/1,1,0/[a2122m- | (2S,3R,4S,5S,6R)-5-amino-3-methoxy-6- |
| | 1a_1-5_2*OC_4*N]/1 | methyloxane-2,4-diol |
| 91854646 | 3,6-Dideoxy-3-amino-beta-D- | (2R,3R,4S,5S,6R)-4-amino-6- |
| | glucopyranose | methyloxane-2,3,5-triol |
| 91854691 | WURCS = 2.0/1,1,0/[a2122m- | (2R,3R,4S,5S,6R)-5-amino-3-methoxy-6- |
| | 1b_1-5_2*OC_4*N]/1 | methyloxane-2,4-diol |
| 91854708 | WURCS = 2.0/1,1,0/[a2211m- | (2R,3R,4R,5R,6S)-5-amino-6- |
| | 1a_1-5_4*N]/1 | methyloxane-2,3,4-triol |
| 91855316 | WURCS = 2.0/1,1,0/[a1122h- | (2S,3S,4S,5S,6S)-5-amino-6- |
| | 1a_1-5_2*OC_4*N]/1 | (hydroxymethyl)-3-methoxyoxane-2,4- |
| | | diol |
| 91856917 | WURCS = 2.0/1,1,0/[a2dd2h- | (2S,3R,6S)-3-amino-6- |
| | 1a_1-5_2*N]/1 | (hydroxymethyl)oxan-2-ol |
| 91861543 | WURCS = 2.0/1,1,0/[a1122m- | (2R,3S,4S,5S,6R)-5-amino-3-methoxy-6- |
| | 1b_1-5_2*OC_4*N]/1 | methyloxane-2,4-diol |
| 92298481 | (2R,3S,4S,5S,6S)-5-amino-6- | (2R,3S,4S,5S,6S)-5-amino-6- |
| | methyloxane-2,3,4-triol | methyloxane-2,3,4-triol |
| 101165551 | N-[3-(2-Methyl-1,3-dioxolane- | (4aR,6S,7S,8R,8aS)-6-methoxy-7-[3-(2- |
| | 2-yl)propyl]-1-O-methyl-4-O,6- | methyl-1,3-dioxolan-2-yl)propylamino]- |
| | O-benzylidene-alpha-D- | 2-phenyl-4,4a,6,7,8,8a- |
| | mannosamine | hexahydropyrano[3,2-d][1,3]dioxin-8-ol |
| 101165553 | N-[3-(2-Methyl-1,3-dioxolane- | (2R,3S,4R,5S,6S)-6-methoxy-5-[3-(2- |
| | 2-yl)propyl]-1-O-methyl-3-O,6- | methyl-1,3-dioxolan-2-yl)propylamino]- |
| | O-dibenzyl-alpha-D- | 4-phenylmethoxy-2- |
| | mannosamine | (phenylmethoxymethyl)oxan-3-ol |
| 101165554 | N-[3-(2-Methyl-1,3-dioxolane- | (2R,3S,4R,5S,6S)-2-(hydroxymethyl)-5- |
| | 2-yl)propyl]-1-O-benzyl-alpha- | [3-(2-methyl-1,3-dioxolan-2- |
| | D-mannosamine | yl)propylamino]-6-phenylmethoxyoxane- |
| | | 3,4-diol |
| 101208731 | 1-O-Methyl-N-acetyl-3-O,6-O- | N-[(2R,3S,4R,5S,6R)-5-hydroxy-2- |
| | dibenzyl-beta-D-mannosamine | methoxy-4-phenylmethoxy-6- |
| | | (phenylmethoxymethyl)oxan-3- |
| | | yl]acetamide |
| 101230876 | 1-O-Amino-N-acetyl-alpha-D- | N-[(2R,3S,4R,5S,6R)-2-aminooxy-4,5- |
| | mannosamine | dihydroxy-6-(hydroxymethyl)oxan-3- |
| | | yl]acetamide |
| 101237631 | N-[1-Oxo-13-[9-(1- | 2-[2-[2-[3-octadecoxy-2-(9-pyren-1- |
| | pyrenyl)nonyloxy]-2,5,8,11,15- | ylnonoxy)propoxy]ethoxy]ethoxy]ethyl |
| | pentaoxatritriacontane-1-yl]- | N-[(2R,3S,4R,5S,6R)-2,4,5-trihydroxy-6- |
| | beta-D-mannosamine | (hydroxymethyl)oxan-3-yl]carbamate |
| 101241224 | N-Acetyl-1-O-isopropyl-3-O,4- | N-[(2S,3S,4R,5S,6R)-4,5- |
| | O,6-O-tribenzyl-alpha-D- | bis(phenylmethoxy)-6- |
| | mannosamine | (phenylmethoxymethyl)-2-propan-2- |
| | | yloxyoxan-3-yl]acetamide |
| 101265229 | N-Acetylmannosamine-6P | [(2R,3S,4R,5S,6R)-5-acetamido-3,4,6- |
| | | trihydroxyoxan-2-yl]methyl dihydrogen |
| | | phosphate |
| 101333264 | 1-(Diethoxyphosphinylmethyl)- | N-[(2R,3S,4R,5S,6R)-2- |
| | 1-deoxy-N-acetyl-beta-D- | [[diethoxy(oxido)phosphaniumyl]methyl]- |
| | mannosamine | 4,5-dihydroxy-6-(hydroxymethyl)oxan- |
| | | 3-yl]acetamide |
| 101333265 | 1-(Diethoxyphosphinylmethyl)- | N-[(4aR,6R,7S,8R,8aS)-6- |
| | 1-deoxy-N-acetyl-4-O,6-O- | [[diethoxy(oxido)phosphaniumyl]methyl]- |
| | benzylidene-beta-D- | 8-hydroxy-2-phenyl-4,4a,6,7,8,8a- |
| | mannosamine | hexahydropyrano[3,2-d][1,3]dioxin-7- |
| | | yl]acetamide |
| 101343655 | D-Mycosamine | (2S,3S,4S,5S,6R)-4-amino-6- |
| | | methyloxane-2,3,5-triol |
| 101480242 | 1-O-Methyl-N-acetyl-3-O-(2-O- | N-[(2S,3R,4R,5S,6R)-2- |
| | methyl-alpha-L- | [(2R,3S,4R,5S,6S)-5-acetamido-4- |
| | rhamnopyranosyl)-4-O-[2- | [(2S,3R,4R,5R,6S)-4,5-dihydroxy-3- |
| | (acetylamino)-2-deoxy-beta-D- | methoxy-6-methyloxan-2-yl]oxy-2- |
| | glucopyranosyl]-alpha-D- | (hydroxymethyl)-6-methoxyoxan-3- |
| | mannosamine | yl]oxy-4,5-dihydroxy-6- |
| | | (hydroxymethyl)oxan-3-yl]acetamide |
| 101480244 | 1-O-Methyl-N-acetyl-3-O- | [(2R,3S,4R,5S,6S)-5-acetamido-3- |
| | benzyl-4-O-(2-phthalimidyl-3- | [(2S,3R,4R,5S,6R)-4,5-diacetyloxy-6- |
| | O,4-O,6-O-triacetyl-2-deoxy- | (acetyloxymethyl)-3-(1,3-dioxoisoindol- |
| | beta-D-glucopyranosyl)-6-O- | 2-yl)oxan-2-yl]oxy-6-methoxy-4- |
| | benzoyl-alpha-D-mannosamine | phenylmethoxyoxan-2-yl]methyl |
| | | benzoate |
| 101480245 | 1-O-Methyl-N,6-O-diacetyl-4- | [(2R,3S,4R,5S,6S)-5-acetamido-3- |
| | O-[2-(acetylamino)-3-O,4-O,6- | [(2S,3R,4R,5S,6R)-3-acetamido-4,5- |
| | O-triacetyl-2-deoxy-beta-D- | diacetyloxy-6-(acetyloxymethyl)oxan-2- |
| | glucopyranosyl]-alpha-D- | yl]oxy-4-hydroxy-6-methoxyoxan-2- |
| | mannosamine | yl]methyl acetate |
| 101480246 | 1-O-Methyl-N,6-O-diacetyl-3- | [(2R,3S,4R,5S,6S)-5-acetamido-3- |
| | O-(2-O-methyl-3-O,4-O- | [(2S,3R,4R,5S,6R)-3-acetamido-4,5- |
| | dibenzyl-alpha-L- | diacetyloxy-6-(acetyloxymethyl)oxan-2- |
| | rhamnopyranosyl)-4-O-[2- | yl]oxy-6-methoxy-4-[(2S,3R,4R,5S,6S)- |
| | (acetylamino)-3-O,4-O,6-O- | 3-methoxy-6-methyl-4,5- |
| | triacetyl-2-deoxy-beta-D- | bis(phenylmethoxy)oxan-2-yl]oxyoxan- |
| | glucopyranosyl]-alpha-D- | 2-yl]methyl acetate |
| | mannosamine | |
| 101683389 | 4-O-beta-D-Glucopyranosyl-D- | (2S,3R,4S,5R)-2-amino-3,5,6-trihydroxy- |
| | mannosamine | 4-[(2S,3R,4S,5S,6R)-3,4,5-trihydroxy-6- |
| | | (hydroxymethyl)oxan-2-yl]oxyhexanal |
| 101701873 | N-(N-Acetylglycyl)-D- | 2-acetamido-N-[(2S,3R,4S,5R)-3,4,5,6- |
| | mannosamine | tetrahydroxy-1-oxohexan-2-yl]acetamide |
| 101701874 | N-[N-(tert-Butoxycarbonyl)-L- | tert-butyl N-[(2S)-1-oxo-3-phenyl-1- |
| | phenylalanyl]-D-mannosamine | [[(2S,3R,4S,5R)-3,4,5,6-tetrahydroxy-1- |
| | | oxohexan-2-yl]amino]propan-2- |
| | | yl]carbamate |
| 101701875 | N-Decanoyl-D-mannosamine | N-[(2S,3R,4S,5R)-3,4,5,6-tetrahydroxy- |
| | | 1-oxohexan-2-yl]decanamide |
| 101701876 | N-(3-Phenylpropionyl)-D- | 3-phenyl-N-[(2S,3R,4S,5R)-3,4,5,6- |
| | mannosamine | tetrahydroxy-1-oxohexan-2- |
| | | yl]propanamide |
| 101701877 | N-(Cbz-Gly-Gly-)-D- | benzyl N-[2-oxo-2-[[2-oxo-2- |
| | Mannosamine | [[(2S,3R,4S,5R)-3,4,5,6-tetrahydroxy-1- |
| | | oxohexan-2- |
| | | yl]amino]ethyl]amino]ethyl]carbamate |
| 101701878 | N-[5-(Dimethylamino)-1- | 5-(dimethylamino)-N-[(2S,3R,4S,5R)- |
| | naphthylsulfonyl]-D- | 3,4,5,6-tetrahydroxy-1-oxohexan-2- |
| | mannosamine | yl]naphthalene-1-sulfonamide |
| 101701879 | N-[5-[[(3aS,4S,6aR)-2- | 5-[(3aS,4S,6aR)-2-oxo-1,3,3a,4,6,6a- |
| | Oxohexahydro-1H-thieno[3,4- | hexahydrothieno[3,4-d]imidazol-4-yl]-N- |
| | d]imidazol]-4-yl]valeryl]-D- | [(2S,3R,4S,5R)-3,4,5,6-tetrahydroxy-1- |
| | mannosamine | oxohexan-2-yl]pentanamide |
| 101963149 | N-Methacryloyl-D- | 2-methyl-N-[(2S,3R,4S,5R)-3,4,5,6- |
| | mannosamine | tetrahydroxy-1-oxohexan-2-yl]prop-2- |
| | | enamide |
| 102086575 | N-Azido 1-O,3-O,4-O,6-O- | NULL |
| | tetraacetyl-alpha-D- | |
| | mannosamine | |
| 102104875 | N-Methacryloyl-alpha-D- | 2-methyl-N-[(2S,3S,4R,5S,6R)-2,4,5- |
| | mannosamine | trihydroxy-6-(hydroxymethyl)oxan-3- |
| | | yl]prop-2-enamide |
| 102104941 | 1-Methylene-N-[2- | N-[(3S,4R,5S,6R)-2-methylidene-4,5- |
| | (trimethylsilyl)ethylsulfonyl]-3- | bis(phenylmethoxy)-6- |
| | O,4-O,6-O-tribenzyl-1-deoxy- | (phenylmethoxymethyl)oxan-3-yl]-2- |
| | alpha-D-mannosamine | trimethylsilylethanesulfonamide |
| 102104942 | 1-(Hydroxymethyl)-N-[2- | N-[(2R,3S,4R,5S,6R)-2-hydroxy-2- |
| | (trimethylsilyl)ethylsulfonyl]-3- | (hydroxymethyl)-4,5- |
| | O,4-O,6-O-tribenzyl-alpha-D- | bis(phenylmethoxy)-6- |
| | mannosamine | (phenylmethoxymethyl)oxan-3-yl]-2- |
| | | trimethylsilylethanesulfonamide |
| 102104945 | 1-O,1- | (5S,6S,7R,8S,9R)-2,2-dimethyl-7,8- |
| | (Dimethylmethyleneoxymethylene)- | bis(phenylmethoxy)-9- |
| | 3-O,4-O,6-O-tribenzyl- | (phenylmethoxymethyl)-1,3,10- |
| | alpha-D-mannosamine | trioxaspiro[4.5]decan-6-amine |
| 102138930 | 1-O-Methyl-N-[(R)-1-thioxo-2- | [(4aR,6S,7S,8R,8aS)-6-methoxy-7- |
| | (tert-butoxycarbonylamino)-3- | [[(2R)-3-methyl-2-[(2-methylpropan-2- |
| | methylbutyl]-3-O-benzoyl-4- | yl)oxycarbonylamino]butanethioyl]amino]- |
| | O,6-O-benzylidene-alpha-D- | 2-phenyl-4,4a,6,7,8,8a- |
| | mannosamine | hexahydropyrano[3,2-d][1,3]dioxin-8-yl] |
| | | benzoate |
| 102138931 | 1-O-Methyl-N-[(S)-1-thioxo-2- | [(4aR,6S,7S,8R,8aS)-6-methoxy-7- |
| | (tert-butoxycarbonylamino)-3- | [[(2S)-3-methyl-2-[(2-methylpropan-2- |
| | methylbutyl]-3-O-benzoyl-4- | yl)oxycarbonylamino]butanethioyl]amino]- |
| | O,6-O-benzylidene-alpha-D- | 2-phenyl-4,4a,6,7,8,8a- |
| | mannosamine | hexahydropyrano[3,2-d][1,3]dioxin-8-yl] |
| | | benzoate |
| 102171134 | WURCS = 2.0/1,1,0/[a2112m- | (2R,3R,4S,5R,6R)-4-amino-6- |
| | 1b_1-5_3*N]/1 | methyloxane-2,3,5-triol |
| 102290495 | N-Acetyl-6-O-[2-(acetylamino)- | disodium; N-[(1S,2R,3R,4R,6S)-6- |
| | 2-deoxy-5-O-carba-alpha-D- | [[(1R,2R,3R,4S,5S)-5-[3- |
| | mannopyranose-1-O- | aminopropoxy(hydroxy)phosphoryl]oxy- |
| | yl(sodiooxy)phosphinyl]-5-O- | 2,3-dihydroxy-4-(1- |
| | carba-alpha-D-mannosamine 1- | oxidoethylideneamino)cyclohexyl]methoxy- |
| | phosphoric acid 3- | hydroxyphosphoryl]oxy-2,3- |
| | aminopropyl = sodium salt | dihydroxy-4- |
| | | (hydroxymethyl)cyclohexyl]ethanimidate |
| 102290498 | N-Acetyl-5-O-carba-alpha-D- | sodium; N-[(1S,2R,3R,4R,6S)-6-[3- |
| | mannosamine 1-phosphoric | aminopropoxy(hydroxy)phosphoryl]oxy- |
| | acid 3-aminopropyl = sodium | 2,3-dihydroxy-4- |
| | salt | (hydroxymethyl)cyclohexyl]ethanimidate |
| 102290499 | 1-O,N-Diacetyl-3-O,4-O- | [(1S,2R,3R,4R,5R)-2-acetamido-5-[[tert- |
| | dibenzyl-6-O-(tert- | butyl(diphenyl)silyl]oxymethyl]-3,4- |
| | butyldiphenylsilyl)-5-O-carba- | bis(phenylmethoxy)cyclohexyl] acetate |
| | alpha-D-mannosamine | |
| 102290502 | 1-O,N-Diacetyl-3-O,4-O- | NULL |
| | dibenzyl-5-O-carba-alpha-D- | |
| | mannosamine 6-phosphonic | |
| | acid triethylammonium salt | |
| 102290506 | 1-O,N-Diacetyl-3-O,4-O- | NULL |
| | dibenzyl-alpha-D-mannosamine | |
| | 6-phosphoric acid 2- | |
| | (acetylamino)-3-O,4-O- | |
| | dibenzyl-6-O-(tert- | |
| | butyldiphenylsilyl)-2-deoxy- | |
| | alpha-D-mannopyranosyl = | |
| | triethylammonium salt | |
| 102521907 | N-Acetyl-5-O-methyl-D- | N-[(2S,3R,4S,5R)-3,4,6-trihydroxy-5- |
| | mannosamine | methoxy-1-oxohexan-2-yl]acetamide |
| 117777158 | (1S,5S)-5-amino-2- | (1S,5S)-5-amino-2-(hydroxymethyl)-3,7- |
| | (hydroxymethyl)-3,7- | dioxabicyclo[4.1.0]heptan-4-ol |
| | dioxabicyclo[4.1.0]heptan-4-ol | |
| 117853811 | (3R,5S,6R)-5-amino-2- | (3R,5S,6R)-5-amino-2-(hydroxymethyl)- |
| | (hydroxymethyl)-6- | 6-methoxyoxane-3,4-diol |
| | methoxyoxane-3,4-diol | |
| 117963066 | (3S,4R,6S)-5-amino-6-ethoxy- | (3S,4R,6S)-5-amino-6-ethoxy-2- |
| | 2-(hydroxymethyl)oxane-3,4- | (hydroxymethyl)oxane-3,4-diol |
| | diol | |
| 117963071 | (3S,5S,6R)-5-amino-2- | (3S,5S,6R)-5-amino-2-(hydroxymethyl)- |
| | (hydroxymethyl)-6- | 6-methoxyoxane-3,4-diol |
| | methoxyoxane-3,4-diol | |
| 117971483 | (3S,4R,6S)-5-amino-2- | (3S,4R,6S)-5-amino-2-(hydroxymethyl)- |
| | (hydroxymethyl)-6- | 6-methoxyoxane-3,4-diol |
| | methoxyoxane-3,4-diol | |
| 118028402 | (2R,3S,5R)-2-(aminomethyl)-6- | (2R,3S,5R)-2-(aminomethyl)-6- |
| | methoxyoxane-3,4,5-triol | methoxyoxane-3,4,5-triol |
| 118052578 | (3S,4R,5S)-5-amino-2- | (3S,4R,5S)-5-amino-2-(hydroxymethyl)- |
| | (hydroxymethyl)-6- | 6-methoxyoxane-3,4-diol |
| | methoxyoxane-3,4-diol | |
| 118054683 | (3R,5S,6R)-5-amino-6-ethoxy- | (3R,5S,6R)-5-amino-6-ethoxy-2- |
| | 2-(hydroxymethyl)oxane-3,4- | (hydroxymethyl)oxane-3,4-diol |
| | diol | |
| 118132059 | (3S,5S,6R)-5-amino-2- | (3S,5S,6R)-5-amino-2-(hydroxymethyl)- |
| | (hydroxymethyl)-3,6- | 3,6-dimethoxyoxan-4-ol |
| | dimethoxyoxan-4-ol | |
| 118332838 | (2R,3S,4S,5R,6S)-3-amino-6- | (2R,3S,4S,5R,6S)-3-amino-6-ethyloxane- |
| | ethyloxane-2,4,5-triol | 2,4,5-triol |
| 118332840 | (2R,3R,4S,5S,6R)-3-amino-6- | (2R,3R,4S,5S,6R)-3-amino-6- |
| | ethyloxane-2,4,5-triol | ethyloxane-2,4,5-triol |
| 118332845 | (2R,3R,4S,5S,6S)-3-amino-6- | (2R,3R,4S,5S,6S)-3-amino-6-ethyloxane- |
| | ethyloxane-2,4,5-triol | 2,4,5-triol |
| 118332851 | (2S,3S,4S,5R,6S)-3-amino-6- | (2S,3S,4S,5R,6S)-3-amino-6-ethyloxane- |
| | ethyloxane-2,4,5-triol | 2,4,5-triol |
| 118332852 | (2S,3R,4R,5S,6R)-3-amino-6- | (2S,3R,4R,5S,6R)-3-amino-6- |
| | ethyloxane-2,4,5-triol | ethyloxane-2,4,5-triol |
| 118332855 | (2S,3S,4S,5S,6R)-3-amino-6- | (2S,3S,4S,5S,6R)-3-amino-6-ethyloxane- |
| | ethyloxane-2,4,5-triol | 2,4,5-triol |
| 118332856 | (2R,3S,4R,5R,6S)-3-amino-6- | (2R,3S,4R,5R,6S)-3-amino-6- |
| | ethyloxane-2,4,5-triol | ethyloxane-2,4,5-triol |
| 118332857 | (2S,3R,4S,5S,6S)-3-amino-6- | (2S,3R,4S,5S,6S)-3-amino-6-ethyloxane- |
| | ethyloxane-2,4,5-triol | 2,4,5-triol |
| 118332858 | (2R,3R,4S,5R,6S)-3-amino-6- | (2R,3R,4S,5R,6S)-3-amino-6- |
| | ethyloxane-2,4,5-triol | ethyloxane-2,4,5-triol |
| 118332860 | (2R,3R,4R,5R,6R)-3-amino-6- | (2R,3R,4R,5R,6R)-3-amino-6- |
| | ethyloxane-2,4,5-triol | ethyloxane-2,4,5-triol |
| 118332861 | (2S,3R,4R,5S,6S)-3-amino-6- | (2S,3R,4R,5S,6S)-3-amino-6-ethyloxane- |
| | ethyloxane-2,4,5-triol | 2,4,5-triol |
| 118332862 | (2R,3R,4R,5S,6S)-3-amino-6- | (2R,3R,4R,5S,6S)-3-amino-6- |
| | ethyloxane-2,4,5-triol | ethyloxane-2,4,5-triol |
| 118332863 | (2R,3R,4S,5R,6R)-3-amino-6- | (2R,3R,4S,5R,6R)-3-amino-6- |
| | ethyloxane-2,4,5-triol | ethyloxane-2,4,5-triol |
| 118332864 | (2S,3S,4R,5S,6R)-3-amino-6- | (2S,3S,4R,5S,6R)-3-amino-6-ethyloxane- |
| | ethyloxane-2,4,5-triol | 2,4,5-triol |
| 118332866 | (2R,3S,4S,5S,6R)-3-amino-6- | (2R,3S,4S,5S,6R)-3-amino-6-ethyloxane- |
| | ethyloxane-2,4,5-triol | 2,4,5-triol |
| 118332867 | (2S,3R,4R,5R,6R)-3-amino-6- | (2S,3R,4R,5R,6R)-3-amino-6- |
| | ethyloxane-2,4,5-triol | ethyloxane-2,4,5-triol |
| 118332868 | (2R,3S,4R,5S,6S)-3-amino-6- | (2R,3S,4R,5S,6S)-3-amino-6-ethyloxane- |
| | ethyloxane-2,4,5-triol | 2,4,5-triol |
| 118332869 | (2S,3S,4R,5R,6R)-3-amino-6- | (2S,3S,4R,5R,6R)-3-amino-6- |
| | ethyloxane-2,4,5-triol | ethyloxane-2,4,5-triol |
| 118332871 | (2S,3R,4S,5S,6R)-3-amino-6- | (2S,3R,4S,5S,6R)-3-amino-6-ethyloxane- |
| | ethyloxane-2,4,5-triol | 2,4,5-triol |
| 118332877 | (2S,3S,4R,5R,6S)-3-amino-6- | (2S,3S,4R,5R,6S)-3-amino-6-ethyloxane- |
| | ethyloxane-2,4,5-triol | 2,4,5-triol |
| 118332881 | (2R,3S,4R,5R,6R)-3-amino-6- | (2R,3S,4R,5R,6R)-3-amino-6- |
| | ethyloxane-2,4,5-triol | ethyloxane-2,4,5-triol |
| 118332883 | (2R,3R,4R,5S,6R)-3-amino-6- | (2R,3R,4R,5S,6R)-3-amino-6- |
| | ethyloxane-2,4,5-triol | ethyloxane-2,4,5-triol |
| 118332884 | (2R,3R,4R,5R,6S)-3-amino-6- | (2R,3R,4R,5R,6S)-3-amino-6- |
| | ethyloxane-2,4,5-triol | ethyloxane-2,4,5-triol |
| 118332886 | (2R,3S,4R,5S,6R)-3-amino-6- | (2R,3S,4R,5S,6R)-3-amino-6- |
| | ethyloxane-2,4,5-triol | ethyloxane-2,4,5-triol |
| 118332888 | (2S,3R,4R,5R,6S)-3-amino-6- | (2S,3R,4R,5R,6S)-3-amino-6- |
| | ethyloxane-2,4,5-triol | ethyloxane-2,4,5-triol |
| 118332889 | (2S,3S,4R,5S,6S)-3-amino-6- | (2S,3S,4R,5S,6S)-3-amino-6-ethyloxane- |
| | ethyloxane-2,4,5-triol | 2,4,5-triol |
| 118332890 | (2S,3R,4S,5R,6R)-3-amino-6- | (2S,3R,4S,5R,6R)-3-amino-6- |
| | ethyloxane-2,4,5-triol | ethyloxane-2,4,5-triol |
| 118332891 | (2S,3R,4S,5R,6S)-3-amino-6- | (2S,3R,4S,5R,6S)-3-amino-6-ethyloxane- |
| | ethyloxane-2,4,5-triol | 2,4,5-triol |
| 118347813 | (3R,5R,6S)-5-amino-2- | (3R,5R,6S)-5-amino-2-(hydroxymethyl)- |
| | (hydroxymethyl)-6- | 6-methoxyoxane-3,4-diol |
| | methoxyoxane-3,4-diol | |
| 118353391 | (2S,4S,5S,6R)-4-amino-6- | (2S,4S,5S,6R)-4-amino-6- |
| | (aminomethyl)oxane-2,5-diol | (aminomethyl)oxane-2,5-diol |
| 118404634 | 5-Amino-2-(hydroxymethyl)- | 5-amino-2-(hydroxymethyl)-4,6- |
| | 4,6-dimethoxyoxan-3-ol | dimethoxyoxan-3-ol |
| 118450094 | (2S,5R)-3-(chloroamino)-6- | (2S,5R)-3-(chloroamino)-6- |
| | (hydroxymethyl)oxane-2,4,5- | (hydroxymethyl)oxane-2,4,5-triol |
| | triol | |
| 118450096 | (3S,6S)-5-amino-2- | (3S,6S)-5-amino-2-(hydroxymethyl)-6- |
| | (hydroxymethyl)-6- | methoxyoxane-3,4-diol |
| | methoxyoxane-3,4-diol | |
| 118592182 | (3R)-5-amino-2- | (3R)-5-amino-2-(hydroxymethyl)-6- |
| | (hydroxymethyl)-6- | methoxyoxane-3,4-diol |
| | methoxyoxane-3,4-diol | |
| 118631943 | (2S,4R,5S)-3-(chloroamino)-6- | (2S,4R,5S)-3-(chloroamino)-6- |
| | (hydroxymethyl)oxane-2,4,5- | (hydroxymethyl)oxane-2,4,5-triol |
| | triol | |
| 121227247 | 2-Amino-2,6-dideoxy-3-O- | (2S,3R,4R,5S,6R)-3-amino-4-methoxy-6- |
| | methyl-alpha-D- | methyloxane-2,5-diol |
| | galactopyranose | |
| 121313220 | (3S,4R,6R)-5-amino-6-ethoxy- | (3S,4R,6R)-5-amino-6-ethoxy-2- |
| | 2-(hydroxymethyl)oxane-3,4- | (hydroxymethyl)oxane-3,4-diol |
| | diol | |
| 122542924 | (1S,4R,5S)-5-amino-2- | (1S,4R,5S)-5-amino-2-(hydroxymethyl)- |
| | (hydroxymethyl)-3,7- | 3,7-dioxabicyclo[4.1.0]heptan-4-ol |
| | dioxabicyclo[4.1.0]heptan-4-ol | |
| 122581532 | (2R,6S)-4-amino-6- | (2R,6S)-4-amino-6-(aminomethyl)oxane- |
| | (aminomethyl)oxane-2,3-diol | 2,3-diol |
| 122590991 | 5-Thio-d-mannosamine | (3S,4R,5S,6R)-3-amino-6- |
| | | (hydroxymethyl)thiane-2,4,5-triol |
| 123898938 | (2S,3R,5R)-3-amino-6- | (2S,3R,5R)-3-amino-6- |
| | (fluoromethyl)oxane-2,4,5-triol | (fluoromethyl)oxane-2,4,5-triol |
| 126541635 | (2R,5S)-5-amino-2- | (2R,5S)-5-amino-2-hydroperoxy-6- |
| | hydroperoxy-6- | (hydroxymethyl)oxan-3-ol |
| | (hydroxymethyl)oxan-3-ol | |
| 126621129 | 3-Amino-6- | 3-amino-6-(methoxymethyl)oxane-2,4,5- |
| | (methoxymethyl)oxane-2,4,5- | triol |
| | triol | |
| 126647738 | (3S,6R)-5-amino-2- | (3S,6R)-5-amino-2-(hydroxymethyl)-3,6- |
| | (hydroxymethyl)-3,6- | dimethoxyoxan-4-ol |
| | dimethoxyoxan-4-ol | |
| 126687604 | (2R,5S)-3-amino-6- | (2R,5S)-3-amino-6-methyloxane-2,4,5- |
| | methyloxane-2,4,5-triol | triol |
| 129088073 | (2S,3R,4S,6R)-5-amino-6- | (2S,3R,4S,6R)-5-amino-6-methyloxane- |
| | methyloxane-2,3,4-triol | 2,3,4-triol |
| 129633488 | 4,6-Dideoxy-4-amino-d- | (3R,4S,5S,6R)-5-(deuterioamino)-6- |
| | glucopyranose | methyloxane-2,3,4-triol |
| 129636252 | [(2R,3S,4R,5S)-5-[(2- | [(2R,3S,4R,5S)-5-[(2- |
| | deuterioacetyl)amino]-3,4,6- | deuterioacetyl)amino]-3,4,6- |
| | trihydroxyoxan-2-yl]methyl | trihydroxyoxan-2-yl]methyl dihydrogen |
| | dihydrogen phosphate | phosphate |
| 129670265 | N-acetyl(- | N-[(3S,4R,5S,6R)-2,4,5-trihydroxy-2-(2- |
| | glycoloyl)mannosamine | hydroxyacetyl)-6-(hydroxymethyl)oxan- |
| | | 3-yl]acetamide |
| 129670973 | d-Mannosamine hydrate | (3S,4R,5S,6R)-3-amino-6- |
| | | (hydroxymethyl)oxane-2,4,5-triol; hydrate |
| 129672133 | 1-Quinovosamine | (3S,4S,5R,6S)-3-amino-6-methyloxane- |
| | | 2,4,5-triol |
| 129694138 | N-acetyl-D-mannosamine-1- | [(3S,4R,5S,6R)-3-[(2- |
| | phosphate | deuterioacetyl)amino]-4,5-dihydroxy-6- |
| | | (hydroxymethyl)oxan-2-yl] dihydrogen |
| | | phosphate |
| 129704038 | 3,6-Anhydrogalactosamine | (1R,4R,5R,8R)-4-amino-2,6- |
| | | dioxabicyclo[3.2.1]octane-3,8-diol |
| 129712089 | 2,3-Dideoxy- | N′-acetamido-N′-[(2S,3S,4S,5S,6R)- |
| | diacetamido | 2,3,4,5-tetrahydroxy-6- |
| | mannosamine | (hydroxymethyl)oxan-2- |
| | | yl]acetohydrazide |
| 129717962 | 4-O-methyl-glucosamine | (3R,4R,5S,6R)-3-amino-6- |
| | | (hydroxymethyl)-5-methoxyoxane-2,4- |
| | | diol |
| 129718019 | Acetyl-d-mannosamine | 1-[(3S,4R,5S,6R)-3-amino-2,4,5- |
| | | trihydroxy-6-(hydroxymethyl)oxan-2-yl]- |
| | | 2-deuterioethanone |
| 129719883 | Dansyl-mannosamine | (3S,4R,5S,6R)-3-amino-2-[5- |
| | | (dimethylamino)naphthalen-1- |
| | | yl]sulfonyl-6-(hydroxymethyl)oxane- |
| | | 2,4,5-triol |
| 129727527 | diphospho-N-acetyl-d- | [(3S,4R,5S,6R)-3-[(2- |
| | mannosamine | deuterioacetyl)amino]-4,5-dihydroxy-6- |
| | | (hydroxymethyl)oxan-2-yl] phosphono |
| | | hydrogen phosphate |
| 129729727 | Methyl 2-amino-2-deoxy-d- | (2R,3S,4S,5S)-5-amino-2- |
| | altropyranoside | (hydroxymethyl)-6-methoxyoxane-3,4- |
| | | diol |
| 129800705 | n-Pentanoyl-d-mannosamine | 5-deuterio-N-[(3S,4R,5S,6R)-2,4,5- |
| | | trihydroxy-6-(hydroxymethyl)oxan-3- |
| | | yl]pentanamide |
| 129810372 | Hydrochloride d-mannosamine | (3S,4R,5S,6R)-3-amino-6- |
| | hydr ochloride | (hydroxymethyl)oxane-2,4,5- |
| | | triol; dihydrochloride |
| 129813217 | N-acetyl-O-acetyl- | [(3S,4R,5S,6R)-3-acetamido-4,5- |
| | mannosamine | dihydroxy-6-(hydroxymethyl)oxan-2-yl] |
| | | acetate |
| 129820114 | 4,6-di-O-methyl-galactosamine | (3R,4R,5R,6R)-3-amino-5-methoxy-6- |
| | | (methoxymethyl)oxane-2,4-diol |
| 129829142 | 3-Thiogalactosamine | (3S,4R,5S,6R)-3-amino-6- |
| | | (hydroxymethyl)-4-sulfanyloxane-2,5- |
| | | diol |
| 129846890 | 3-O-methylgalactosamine | (3R,4R,5R,6R)-3-amino-6- |
| | | (hydroxymethyl)-4-methoxyoxane-2,5- |
| | | diol |
| 129847410 | 2,3-Diacetamido mannosamine | N-[(3R,4R,5R,6R)-3-acetamido-3-amino- |
| | | 2,4,5-trihydroxy-6-(hydroxymethyl)oxan- |
| | | 4-yl]acetamide |
| 129849795 | 1,6-Anhydro glucosamine | (1R,2S,3R,4R)-4-amino-6,8- |
| | | dioxabicyclo[3.2.1]octane-2,3-diol |
| 129850818 | Methyl 4-amino-4,6- | (3S,4S,5S,6R)-5-amino-2-methoxy-6- |
| | dideoxymannopyranoside | methyloxane-3,4-diol |
| 129850915 | N-methoxyacetyl-d- | 2-deuterio-2-methoxy-N-[(3S,4R,5S,6R)- |
| | mannosamine | 2,4,5-trihydroxy-6-(hydroxymethyl)oxan- |
| | | 3-yl]acetamide |
| 129850926 | N-azidoacetyl-d-mannosamine | 2-azido-2-deuterio-N-[(3S,4R,5S,6R)- |
| | | 2,4,5-trihydroxy-6-(hydroxymethyl)oxan- |
| | | 3-yl]acetamide |
| 129850927 | N-acetoxyacetyl-d- | [1-deuterio-2-oxo-2-[[(3S,4R,5S,6R)- |
| | mannosamine | 2,4,5-trihydroxy-6-(hydroxymethyl)oxan- |
| | | 3-yl]amino]ethyl] acetate |
| 129851298 | n-Phenylacetyl-d-mannosamine | 2-deuterio-2-phenyl-N-[(3S,4R,5S,6R)- |
| | | 2,4,5-trihydroxy-6-(hydroxymethyl)oxan- |
| | | 3-yl]acetamide |
| 129854893 | 6-O-methylgalactosamine | (3R,4R,5R,6R)-3-amino-6- |
| | | (methoxymethyl)oxane-2,4,5-triol |
| 129856562 | N-propyl-d-mannosamine | (3S,4R,5S,6R)-3-(3- |
| | | deuteriopropylamino)-6- |
| | | (hydroxymethyl)oxane-2,4,5-triol |
| 129858975 | 1,3,4,6-tetra-O-acetyl-N- | [(2R,3S,4R,5S)-3,4,6-triacetyloxy-5-[(2- |
| | azidoacetyl-d-mannosamine | azido-2-deuterioacetyl)amino]oxan-2- |
| | | yl]methyl acetate |
| 129878031 | n-Butanoyl-d-mannosamine | 4-deuterio-N-[(3S,4R,5S,6R)-2,4,5- |
| | | trihydroxy-6-(hydroxymethyl)oxan-3- |
| | | yl]butanamide |
| 129878068 | N-(3-cyano- | 3-cyano-N-[(3S,4R,5S,6R)-2,4,5- |
| | propanoyl)mannosamine | trihydroxy-6-(hydroxymethyl)oxan-3- |
| | | yl]propanamide |
| 130753326 | (3R,4R,5S,6R)-3-amino-6- | (3R,4R,5S,6R)-3-amino-6- |
| | (hydroxymethyl)-4,5- | (hydroxymethyl)-4,5-dimethoxyoxan-2- |
| | dimethoxyoxan-2-ol | ol |
| 130764364 | alpha-D-Fucosaminepyranose | (2S,3R,4R,5R,6R)-3-amino-6- |
| | | methyloxane-2,4,5-triol |
| 130817392 | (2R,3R,4R,5R,6R)-5-amino-2- | (2R,3R,4R,5R,6R)-5-amino-2- |
| | (hydroxymethyl)-6- | (hydroxymethyl)-6-methoxyoxane-3,4- |
| | methoxyoxane-3,4-diol | diol |
| 130873921 | (3S,4R,5R,6R)-3-amino-6- | (3S,4R,5R,6R)-3-amino-6- |
| | (hydroxymethyl)-4- | (hydroxymethyl)-4-sulfanyloxane-2,5- |
| | sulfanyloxane-2,5-diol | diol |
| 130896032 | (3R,4R,5S,6R)-3-amino-4- | (3R,4R,5S,6R)-3-amino-4-methoxy-6- |
| | methoxy-6- | (methoxymethyl)oxane-2,5-diol |
| | (methoxymethyl)oxane-2,5-diol | |
| 130961740 | (3R,4S,5S,6R)-6- | (3R,4S,5S,6R)-6-(aminomethyl)-3- |
| | (aminomethyl)-3-fluorooxane- | fluorooxane-2,4,5-triol |
| | 2,4,5-triol | |
| 131028255 | Glucopyranoside, methyl 2,3- | (2R,3S,4R,5R,6S)-4,5-diamino-2- |
| | diamino-2,3-dideoxy-, alpha-D- | (hydroxymethyl)-6-methoxyoxan-3-ol |
| 131124130 | (1R,2S,3R,4S,5R)-4-amino-6,8- | (1R,2S,3R,4S,5R)-4-amino-6,8- |
| | dioxabicyclo[3.2.1]octane-2,3- | dioxabicyclo[3.2.1 ]octane-2,3-diol |
| | diol | |
| 131195978 | (3R,4R,5R,6S)-3-amino-6- | (3R,4R,5R,6S)-3-amino-6- |
| | (fluoromethyl)oxane-2,4,5-triol | (fluoromethyl)oxane-2,4,5-triol |
| 131734108 | N-benzyl-mannosamine | (3S,4R,5S,6R)-3-(benzylamino)-6- |
| | | (hydroxymethyl)oxane-2,4,5-triol |
| 131735387 | 1,3,4,6-Tetra-o-acetyl-n-(4-oxo- | [(2R,3S,4R,5S)-3,4,6-triacetyloxy-5-(4- |
| | hexanoyl)-d-mannosamine | oxohexanoylamino)oxan-2-yl]methyl |
| | | acetate |
| 131742298 | 1,3,4,6-tetra-O-acety-D- | [(2R,3S,4R,5S)-3,4,6-triacetyloxy-5- |
| | mannosamine TFA salt | aminooxan-2-yl]methyl acetate; 2,2,2- |
| | | trifluoroacetic acid |
| 131742300 | 1,3,4,6-tetra-O-acety-N-Boc-D- | [(2R,3S,4R,5S)-3,4,6-triacetyloxy-5-[(2- |
| | mannosamine | methylpropan-2- |
| | | yl)oxycarbonylamino]oxan-2-yl]methyl |
| | | acetate |
| 131841589 | N-acetyl-D-mannosamine 1- | [(3S,4R,5S,6R)-3-acetamido-4,5- |
| | phosphate | dihydroxy-6-(hydroxymethyl)oxan-2-yl] |
| | | phosphate |
| 132515829 | N-(1-Oxo-4-pentyne-1-yl)-beta- | N-[(2R,3S,4R,5S,6R)-2,4,5-trihydroxy-6- |
| | D-mannosamine | (hydroxymethyl)oxan-3-yl]pent-4- |
| | | ynamide |
| 133126553 | n-Acetyl mannosamine | N-[(2S,3R,4S,5R,6S)-2,4,5-trihydroxy-6- |
| | | (hydroxymethyl)oxan-3- |
| | | yl]acetamide; hydrate |
| 134169471 | (2S,3R,6R)-5-amino-6- | (2S,3R,6R)-5-amino-6-methyloxane- |
| | methyloxane-2,3,4-triol | 2,3,4-triol |
| 134274956 | (2R,3R,6S)-3-amino-6- | (2R,3R,6S)-3-amino-6- |
| | (hydroxymethyl)oxan-2-ol | (hydroxymethyl)oxan-2-ol |
| 134516483 | [3-Amino-2,5-dihydroxy-6- | [3-amino-2,5-dihydroxy-6- |
| | (hydroxymethyl)oxan-4-yl] | (hydroxymethyl)oxan-4-yl] hypoiodite |
| | hypoiodite | |
| 134694276 | beta-L-Galactopyranose, 2- | (2S,3S,4S,5S,6S)-3-amino-6- |
| | amino-2,6-dideoxy- | methyloxane-2,4,5-triol |
| 134694326 | alpha-L-Fucosaminepyranose | (2R,3S,4S,5S,6S)-3-amino-6- |
| | | methyloxane-2,4,5-triol |
| 134824447 | (2S,3S,4R,5S)-3-Amino-5- | (2S,3S,4R,5S)-3-amino-5-[(1R)-1,2- |
| | ((1R)-1,2- | dihydroxyethyl]oxolane-2,4-diol |
| | dihydroxyethyl)tetrahydrofuran- | |
| | 2,4-diol | |
| 134824490 | (2R,3S,4R,5S)-3-Amino-5- | (2R,3S,4R,5S)-3-amino-5-[(1R)-1,2- |
| | ((1R)-1,2- | dihydroxyethyl]oxolane-2,4-diol |
| | dihydroxyethyl)tetrahydrofuran- | |
| | 2,4-diol | |
| 134855699 | (3S,6S)-2-(aminomethyl)-6- | (3S,6S)-2-(aminomethyl)-6- |
| | methoxyoxane-3,4,5-triol | methoxyoxane-3,4,5-triol |
| 139614694 | (3S,5S,6R)-3-amino-2-deuterio- | (3S,5S,6R)-3-amino-2-deuterio-6- |
| | 6-(hydroxymethyl)oxane-2,5- | (hydroxymethyl)oxane-2,5-diol |
| | diol | |
| 140355716 | (1R,4R,5R,8S)-4-amino-2,6- | (1R,4R,5R,8S)-4-amino-2,6- |
| | dioxabicyclo[3.2.1]octane-3,8- | dioxabicyclo[3.2.1]octane-3,8-diol |
| | diol | |
| 140380451 | (3R,4R,5S,6R)-3-amino-5- | (3R,4R,5S,6R)-3-amino-5-fluoro-6- |
| | fluoro-6- | (hydroxymethyl)oxane-2,4-diol |
| | (hydroxymethyl)oxane-2,4-diol | |
| 140542575 | (3R,4S,6S)-3-amino-6- | (3R,4S,6S)-3-amino-6- |
| | (hydroxymethyl)oxane-2,4-diol | (hydroxymethyl)oxane-2,4-diol |
| 140568801 | [(2R,4S,5S)-2,4,5-trihydroxy-6- | [(2R,4S,5S)-2,4,5-trihydroxy-6- |
| | (hydroxymethyl)oxan-3- | (hydroxymethyl)oxan-3-yl]azanide |
| | yl]azanide | |
| 140663408 | CID 140663408 | NULL |
| 140674981 | (3R,4R,5R,6R)-3-amino-2,6- | (3R,4R,5R,6R)-3-amino-2,6- |
| | dimethyloxane-2,4,5-triol | dimethyloxane-2,4,5-triol |
| 140675205 | [(2R,4R,5S)-4-hydroxy-6- | [(2R,4R,5S)-4-hydroxy-6- |
| | (hydroxymethyl)-2,5- | (hydroxymethyl)-2,5-dimethoxyoxan-3- |
| | dimethoxyoxan-3-yl]azanium | yl]azanium |
| 140867786 | (3S,4R,5R,6R)-3-amino-4- | (3S,4R,5R,6R)-3-amino-4-fluoro-6- |
| | fluoro-6- | (hydroxymethyl)oxane-2,5-diol |
| | (hydroxymethyl)oxane-2,5-diol | |
| 141541850 | (2R,3S,4R,5R)-5-amino-2- | (2R,3S,4R,5R)-5-amino-2- |
| | [deuterio(hydroxy)methyl]-6- | [deuterio(hydroxy)methyl]-6- |
| | methoxyoxane-3,4-diol | methoxyoxane-3,4-diol |
| 141562795 | (3S,4R,5S,6R)-3-amino-4- | (3S,4R,5S,6R)-3-amino-4- |
| | (deuteriomethyl)-6- | (deuteriomethyl)-6- |
| | (hydroxymethyl)oxane-2,5-diol | (hydroxymethyl)oxane-2,5-diol |
| 141562796 | (3S,4R,5S,6R)-3-amino-4- | (3S,4R,5S,6R)-3-amino-4- |
| | (deuteriomethoxy)-6- | (deuteriomethoxy)-6- |
| | (hydroxymethyl)oxane-2,5-diol | (hydroxymethyl)oxane-2,5-diol |
| 141912369 | (4S)-3,5-diamino-2- | (4S)-3,5-diamino-2-(hydroxymethyl)-6- |
| | (hydroxymethyl)-6- | methoxyoxan-4-ol |
| | methoxyoxan-4-ol | |
| 142579295 | (2S,5S)-6-(aminomethyl)oxane- | (2S,5S)-6-(aminomethyl)oxane-2,4,5- |
| | 2,4,5-triol | triol |
| 142741136 | (2R,3R,4R,5S,6R)-3-amino-6- | (2R,3R,4R,5S,6R)-3-amino-6- |
| | (hydroxymethyl)-5- | (hydroxymethyl)-5-methoxyoxane-2,4- |
| | methoxyoxane-2,4-diol | diol |
| 143143237 | (2S,4R,5S,6R)-6- | (2S,4R,5S,6R)-6-(aminomethyl)oxane- |
| | (aminomethyl)oxane-2,4,5-triol | 2,4,5-triol |
| 143420897 | (3R,4R,5S,6R)-3-amino-6- | (3R,4R,5S,6R)-3-amino-6- |
| | (hydroxymethyl)-4- | (hydroxymethyl)-4-methyloxane-2,5-diol |
| | methyloxane-2,5-diol | |
| 144091923 | (2S,4R,5S)-6- | (2S,4R,5S)-6-(aminomethyl)oxane-2,4,5- |
| | (aminomethyl)oxane-2,4,5-triol | triol |
| 144420235 | (3S,6R)-5-amino-2- | (3S,6R)-5-amino-2-(hydroxymethyl)-4,6- |
| | (hydroxymethyl)-4,6- | dimethoxyoxan-3-ol |
| | dimethoxyoxan-3-ol | |
| 144912620 | (3R,6S)-5-amino-2- | (3R,6S)-5-amino-2-(hydroxymethyl)-6- |
| | (hydroxymethyl)-6- | methoxyoxane-3,4-diol |
| | methoxyoxane-3,4-diol | |
| 144942620 | (5-Amino-4,6-dihydroxyoxan- | (5-amino-4,6-dihydroxyoxan-2-yl)methyl |
| | 2-yl)methyl hypofluorite | hypofluorite |
| 145440171 | 4-Amino-6- | 4-amino-6-(hydroxymethyl)oxane-2,5- |
| | (hydroxymethyl)oxane-2,5-diol | diol |
| 145675032 | (2R,3R,4R,5R,6R)-3-amino-6- | (2R,3R,4R,5R,6R)-3-amino-6- |
| | (hydroxymethyl)-4- | (hydroxymethyl)-4-methyloxane-2,5-diol |
| | methyloxane-2,5-diol | |
| 145723850 | [(4S,6S)-4,5,6-trihydroxyoxan- | [(4S,6S)-4,5,6-trihydroxyoxan-2- |
| | 2-yl]methylazanium | yl]methylazanium |
| 146279042 | (3R,4R,5S,6S)-3-amino-6- | (3R,4R,5S,6S)-3-amino-6- |
| | (fluoromethyl)oxane-2,4,5-triol | (fluoromethyl)oxane-2,4,5-triol |
| 147097207 | (3S,4S,5R,6S)-2- | (3S,4S,5R,6S)-2-(aminomethyl)-6- |
| | (aminomethyl)-6- | methoxyoxane-3,4,5-triol |
| | methoxyoxane-3,4,5-triol | |
| 147205988 | (2S,5S,6S)-3-amino-6- | (2S,5S,6S)-3-amino-6- |
| | (aminomethyl)oxane-2,5-diol | (aminomethyl)oxane-2,5-diol |
| 153377161 | (2R,3S,5R,6R)-3-amino-4- | (2R,3S,5R,6R)-3-amino-4-fluoro-6- |
| | fluoro-6- | (hydroxymethyl)oxane-2,5-diol |
| | (hydroxymethyl)oxane-2,5-diol | |
| 153377197 | (2R,3S,5R,6R)-3-amino-6- | (2R,3S,5R,6R)-3-amino-6- |
| | (hydroxymethyl)-4- | (hydroxymethyl)-4-sulfanyloxane-2,5- |
| | sulfanyloxane-2,5-diol | diol |
| 153377198 | (2R,3R,4R,6R)-3-amino-5- | (2R,3R,4R,6R)-3-amino-5-fluoro-6- |
| | fluoro-6- | (hydroxymethyl)oxane-2,4-diol |
| | (hydroxymethyl)oxane-2,4-diol | |
| 153471493 | (2R,4R,5R)-3-(chloroamino)-6- | (2R,4R,5R)-3-(chloroamino)-6- |
| | (hydroxymethyl)oxane-2,4,5- | (hydroxymethyl)oxane-2,4,5-triol |
| | triol | |
| 153711556 | (2R,3S,4R,5R)-5-amino-6- | (2R,3S,4R,5R)-5-amino-6-deuterio-2- |
| | deuterio-2-(hydroxymethyl)-6- | (hydroxymethyl)-6-methoxyoxane-3,4- |
| | methoxyoxane-3,4-diol | diol |
| 153823937 | (3R,4S,5S,6R)-6- | (3R,4S,5S,6R)-6-(aminomethyl)-2- |
| | (aminomethyl)-2-deuterio-2- | deuterio-2-methoxyoxane-3,4,5-triol |
| | methoxyoxane-3,4,5-triol | |
| 155259579 | (3R,5S,6R)-3-amino-6- | (3R,5S,6R)-3-amino-6-methyloxane-2,5- |
| | methyloxane-2,5-diol | diol |
| 155632859 | (3S,5S)-5-amino-2- | (3S,5S)-5-amino-2-(hydroxymethyl)-3,6- |
| | (hydroxymethyl)-3,6- | dimethoxyoxan-4-ol |
| | dimethoxyoxan-4-ol | |
| 155635728 | (5-Amino-3,6-dimethoxyoxan- | (5-amino-3,6-dimethoxyoxan-2- |
| | 2-yl)methanol | yl)methanol |
| 156615326 | WURCS = 2.0/1,1,0/[a2112m- | (2R,3R,4S,5R,6R)-5-amino-6- |
| | 1b_1-5_4*N]/1 | methyloxane-2,3,4-triol |
| 156615518 | WURCS = 2.0/1,1,0/[a2221m- | (2R,3R,4R,5S,6S)-5-amino-6- |
| | 1a_1-5_4*N]/1 | methyloxane-2,3,4-triol |
| 156615634 | WURCS = 2.0/1,1,0/[a2d2xh- | (3R,5S)-3-amino-6-(aminomethyl)oxane- |
| | 1x_1-5_2*N_6*N]/1 | 2,5-diol |
| 156621412 | tetraacetyl-N-azidoacetyl-D- | [(2R,3S,4R,5S)-3,4,6-triacetyloxy-5- |
| | mannosamine | (carbonazidoylamino)oxan-2-yl]methyl |
| | | acetate |
| 156818509 | (4R,5R,6R)-6- | (4R,5R,6R)-6-(aminomethyl)oxane- |
| | (aminomethyl)oxane-2,4,5-triol | 2,4,5-triol |
| 156818510 | (2R,3R,4R,5S,6R)-5-amino-2- | (2R,3R,4R,5S,6R)-5-amino-2- |
| | (hydroxymethyl)-6- | (hydroxymethyl)-6-methoxyoxane-3,4- |
| | methoxyoxane-3,4-diol | diol |
| 157815237 | [(3S,6R)-5-amino-3,6- | [(3S,6R)-5-amino-3,6-dimethoxyoxan-2- |
| | dimethoxyoxan-2-yl]methanol | yl]methanol |
| 158609400 | (2R,3S,5R,6R)-2- | (2R,3S,5R,6R)-2-(aminomethyl)-6- |
| | (aminomethyl)-6- | methoxyoxane-3,4,5-triol |
| | methoxyoxane-3,4,5-triol | |
| 159993684 | (1R,2S,3R,4S)-4-amino-6,8- | (1R,2S,3R,4S)-4-amino-6,8- |
| | dioxabicyclo[3.2.1]octane-2,3- | dioxabicyclo[3.2.1]octane-2,3-diol |
| | diol | |
| 163455315 | (5R)-2-(aminomethyl)-6- | (5R)-2-(aminomethyl)-6-methoxyoxane- |
| | methoxyoxane-3,4,5-triol | 3,4,5-triol |
| 163815969 | (2R,5S)-6-(hydroxymethyl)-3- | (2R,5S)-6-(hydroxymethyl)-3- |
| | (iodoamino)oxane-2,4,5-triol | (iodoamino)oxane-2,4,5-triol |
| 163827689 | (2S,5S)-5-amino-6- | (2S,5S)-5-amino-6- |
| | (hydroxymethyl)oxane-2,3-diol | (hydroxymethyl)oxane-2,3-diol |
| 163993265 | (5R)-5-amino-2- | (5R)-5-amino-2-(hydroxymethyl)-6- |
| | (hydroxymethyl)-6- | methoxyoxane-3,4-diol |
| | methoxyoxane-3,4-diol | |
| 163993266 | (5S)-5-amino-2- | (5S)-5-amino-2-(hydroxymethyl)-6- |
| | (hydroxymethyl)-6- | methoxyoxane-3,4-diol |
| | methoxyoxane-3,4-diol | |
| 164071574 | (2R,5S)-3-amino-5-methoxy-6- | (2R,5S)-3-amino-5-methoxy-6- |
| | methyloxane-2,4-diol | methyloxane-2,4-diol |
| 164153133 | (4S,6S)-5-amino-2- | (4S,6S)-5-amino-2-(hydroxymethyl)-3,6- |
| | (hydroxymethyl)-3,6- | dimethoxyoxan-4-ol |
| | dimethoxyoxan-4-ol | |
| 165111698 | 1,3,4,6-Tetra-O-acetyl-N-5-(4- | [(2R,3S,4R,5S)-3,4,6-triacetyloxy-5-[(4- |
| | azido-2,3,5,6- | azido-2,3,5,6- |
| | tetrafluorobenzoyl)mannosamine | tetrafluorobenzoyl)amino]oxan-2- |
| | | yl]methyl acetate |
| 165348535 | Methyl 2,3-diamino-2,3- | (2R,3S,4S,5R)-4,5-diamino-2- |
| | dideoxy-D-allopyranoside | (hydroxymethyl)-6-methoxyoxan-3-ol |
| 165412649 | N-Acetyl-D-mannosamine-18O | N-[(2S,3R,4S,5R)-3,4,5,6-tetrahydroxy- |
| | | 1-oxohexan-2-yl]acetamide |
| 165412650 | N-Acetyl-D-mannosamine-13C | N-[(2S,3R,4S,5R)-3,4,5,6-tetrahydroxy- |
| | | 1-oxohexan-2-yl]acetamide |
| 165412651 | N-Acetyl-D-mannosamine- | N-[(2S,3R,4S,5R)-3,4,5,6-tetrahydroxy- |
| | 13C-1 | 1-oxohexan-2-yl]acetamide |
| 165412652 | N-Acetyl-D-mannosamine- | 2,2,2-trideuterio-N-[(2S,3R,4S,5R)- |
| | 18O,d3 | 3,4,5,6-tetrahydroxy-1-oxohexan-2- |
| | | yl]acetamide |
| 165412653 | N-Acetyl-D-mannosamine-d3 | 2,2,2-trideuterio-N-[(2S,3R,4S,5R)- |
| | | 3,4,5,6-tetrahydroxy-1-oxohexan-2- |
| | | yl]acetamide |
| 165412654 | N-Acetyl-D-mannosamine-15N | N-[(2S,3R,4S,5R)-3,4,5,6-tetrahydroxy- |
| | | 1-oxohexan-2-yl]acetamide |

Combination with Known Stroke Treatments

In some embodiments, methods of prevention, attenuation, or treatment of stroke in a subject in need thereof comprising administering a pharmaceutical composition described herein further comprise administering one or more known stroke treatments to the subject. In some embodiments, the one or more known stroke treatments is an anticoagulant medication, a tissue plasminogen activator medication, an antiplatelet medication, or any combination thereof. In some embodiments, the anticoagulant medication is rivaroxaban (Xarelto®), dabigatran (Pradaxa®), apixaban (Eliquis®), or edoxaban (Lixiana®). In some embodiments, the tissue plasminogen activator medication is alteplase (Activase®), reteplase (Retavase®), or tenecteplase (TNKase®). In some embodiments, the antiplatelet medication is acetylsalicylic acid (ASA) given at 75-325 mg/day, clopidogrel, ASA and extended-release dipyridamole, or ticagrelor.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the present disclosure. Although any methods and materials similar or equivalent to those described herein can be used in the practice of the present disclosure, the exemplary methods, devices, and materials are described herein.

The present disclosure provides pharmaceutical compositions and methods for treating an ischemic condition in a subject, including administering to the subject in need thereof an effective amount of hexosamine D-mannosamine (ManN). Because ManN is converted to ManN-6-phosphate (ManN-6p) in vivo, the present disclosure provides for the use of such metabolic precursors and derivatives.

ManN is a hexosamine with an ability to inhibit protein post translational modifications, activate stress pathways and show additivity with VEGF in promoting endothelia; cell (EC) proliferation and angiogenesis. Effects of ManN on ECs and angiogenesis have not been previously reported. Without being bound by theory, using well-known glycosylation inhibitors together with ManN may result in a link between changes in glycosylation patterns in mammalian ECs and angiogenesis. The effects of ManN on endothelial cells may be independent of VEGFR2 activation.

ManN was discovered in the 1960s as a bacterial wall component, and accounts for 5-10% of capsular polysaccharides. The related N-acetyl mannosamine is thought to be an intermediate in the biosynthesis of sialic acids. Over the years, multiple effects of ManN on enzymes, growth factor-mediated signaling, protein stability and cell viability were documented. Most of these effects were not unique to ManN and could be elicited by other hexosamines. In addition, they required high concentrations. ManN was reported to have antitumor properties, to stimulate osteogenic differentiation and to protect articular cartilage. More recently, ManN was used as an intermediate in modifying various molecules/nanoparticles and in the synthesis of non-natural ManNAc analogs for the expression of thiols on cell-surface sialic acids to facilitate high-throughput screening. However, to date no effects of ManN on ECs have been described.

ManN had been previously reported to affect formation of lipid-linked oligosaccharides (LLO) in MDCK cells possibly by inhibiting the a-1,2-mannosyl transferases. Upon ManN treatment, major oligosaccharides associated with the dolichol were Man5GlcNAc2 and Man6GlcNAc2 rather than Glc3Man9GlcNAc2 which was normally found in MDCK cells. In addition, ManN was reported to change protein GPI biosynthesis and hybrid glycans production in the ER. However, none of the angiogenic-related proteins previously examined are GPI-anchored. Without being bound by theory, a decrease in Man-9 could be a direct result of inhibiting LLO donor synthesis, i.e. Glc3Man9GlcNAc20PP-Dol formation, which is then transferred from the dolichol donor onto the polypeptide. In some embodiments, Man-5 may be significantly increased over 24 hours by treating cells with 40 μM ManN. ManN may be not affect a-mannosidases in the ER.

Activation of PI3K-AKT, PLCy-ERK and p38 is associated with VEGFR2-mediated EC survival, proliferation and migration. Other cellular metabolic stress sensors, such as AMPK (AMP-activated protein kinase), could also confer stress adaptation and promote EC survival via eNOS. Without being bound by theory, ERK, AKT, mTOR, AMPKa, eNOS, and ACC activation is a general phenomenon for hexosamines and mannose. However, activation of the JNK/c-Jun and UPR pathways in BCECs is unique to ManN as well as the glycosylation inhibitors. Glycosylation is required for correct protein folding in the ER. A link between LLO inhibition and activation of UPR has been reported. In fact, notwithstanding the complexity of ManN actions, LLO inhibition, followed by UPR activation, seems a plausible explanation for the reported ManN effects.

ECs are able to cope with acute/minor ER stress resulting from glycosylation inhibition by activating the UPR pathway. UPR detects misfolded proteins accumulated in the ER and initiates a response to maintain cellular homeostasis via induction of Bip, a major ER chaperon protein. BiP binds to hydrophobic patches exposed on nascent or incompletely folded proteins that are often non-glycosylated. ManN exhibits a strong induction of Bip expression relative to hexosamines. Similar effects on stress pathway activation may result from glycosylation inhibitors Kif and Cas.

Glycosylation inhibition is thought to be a new pharmacological strategy targeting metabolic pathways essential for excessive angiogenesis in various pathological conditions, and glycosylation inhibitors are expected to have anti-angiogenic and anti-metastatic properties. Glycosylation has been shown to be involved in cellular stress response and compensatory angiogenesis in response to VEGF-VEGFR2 signaling blockade. Stress-induced O-GlcNAcylation was previously reported to promote survival in response to DNA damage, ER stress, glucose deprivation and hypoxia in a variety of cell types. Without being bound by theory, glycosylation inhibition may be linked to angiogenesis promotion, and inhibiting glycosylation within the tumor microenvironment may result in stimulation rather than suppression of tumor angiogenesis.

In some embodiments, ManN may be used to promote angiogenesis in a mouse skin injury model, accompanied by accelerated wound closure. In some embodiments, ManN may be used to stimulate angiogenesis and blood flow recovery in ischemic hindlimbs of mice. Combinations of ManN, or other glycosylation inhibitors, with VEGF-A, may have advantages over monotherapy for the treatment of ischemic disorders. A lack of direct permeability-enhancing effects of ManN may result in less edematous tissues. In this context, damage to lung endothelium is a central pathogenic event in the respiratory failure associated with a variety of infections, including SARS-COV-2. An endothelial cell mitogen like ManN, devoid of permeabilizing effects, may help protect and stabilize blood vessels and thus limit tissue damage.

In some embodiments, intravitreal administration of ManN may be used to enhance retinal neovascularization, for example, in therapeutic applications in ocular diseases. 10-15% of patients with intermediate AMD progress to the neovascular form, while the remaining patients may develop geographic atrophy (GA). Previous studies have shown that loss of choroid capillaries is frequently detected in GA, which raises the possibility that regeneration/protection of choroid capillaries may be a strategy for GA treatment. In some embodiments, intravitreal administration of a therapeutically effective amount of ManN may be used in a method to treat dry age-related macular degeneration (AMD) and/or geographic atrophy (GA), or a combination thereof. In some embodiments, intravitreal administration of a therapeutically effective amount of ManN may be used in a method to treat dry age-related macular degeneration (AMD), diabetic macular edema, macular edema from retinal vein occlusion, diabetic retinopathy, retinal vein occlusion, retinopathy of prematurity, retinal neovascularization in diabetes, optic nerve neovascularization in diabetes, familial exudative vitreoretinopathy, sickle cell disease, or a combination thereof. In some embodiments, intravitreal administration of a therapeutically effective amount of ManN may be used in a method to treat wet age-related macular degeneration (AMD), polypoidal choroidal vasculopathy (PCV), degenerate (pathologic) myopia, or a combination thereof. In some embodiments, intravitreal administration of a therapeutically effective amount of ManN may be used in a method to treat wet age-related macular degeneration (AMD), choroidal neovascularization (CNV), polypoidal choroidal vasculopathy, degenerative (pathologic) myopia, giant cell arteritis, or a combination thereof. In some embodiments, intravitreal administration of a therapeutically effective amount of ManN may be used in a method to treat degenerative (pathologic) myopia. In some embodiments, intravitreal administration of a therapeutically effective amount of ManN slows the progression of the ocular disease. In some embodiments, intravitreal administration of a therapeutically effective amount of ManN halts the progression of the ocular disease. The halting or slowing of the progression of the disease can be mediated by increased non-leaking or minimally-leaking retinal perfusion and/or non-leaking or minimally-leaking retinal revascularization. The halting or slowing of the progression of the disease can be mediated by increased non-leaking or minimally-leaking choroidal perfusion and/or non-leaking or minimally-leaking choroidal revascularization.

In some embodiments, the administration is effective to promote endothelial cell proliferation and angiogenesis in the subject. In some embodiments, the method further includes administering to the subject in need thereof an effective amount of an N-glycosylation inhibitor. In some embodiments, the method further includes administering to the subject in need thereof an effective amount of VEGF.

In some embodiments, the ischemic condition is caused by a disease or a trauma. The present disclosure provides for treatment of a number of conditions characterized by reduced perfusion, including but not limited to diabetic ulcers, macular degeneration, peripheral arterial disease (PAD), limb ischemia, brain or cerebral ischemia, and coronary ischemia.

In some embodiments, the administration is oral, intravenous, intrathecal, or intraperitoneal.

In some embodiments, the present disclosure provides pharmaceutical compositions and methods for inducing angiogenesis in a subject, including administering to the subject in need thereof an effective amount of hexosamine D-mannosamine (ManN).

In some embodiments, the administration is effective to reduce ischemia in the subject. In some embodiments, the ischemia may include brain ischemia. The administration may be effective in preventing, reducing, or treating conditions associated with brain ischemia, such as edema, ischemic stroke, or infarctions. In some embodiments, the method further includes administering to the subject in need thereof an effective amount of an N-glycosylation inhibitor. In some embodiments, the method further includes administering to the subject in need thereof an effective amount of VEGF.

In some embodiments, the subject is in need of inducing angiogenesis due to an ischemic condition is caused by a disease or a trauma.

In some embodiments, the administration is oral, intravenous, intrathecal, or intraperitoneal.

In some embodiments, the present disclosure provides pharmaceutical compositions and methods for inhibiting protein glycosylation in a cell, including administering to the cell an effective amount of hexosamine D-mannosamine (ManN).

In some embodiments, the administration is in vivo. In some embodiments, the administration is ex vivo. In some embodiments, the administration is effective to stimulate EC proliferation and angiogenesis. In some embodiments, the administration is effective to activate JNK and an unfolded protein response caused by ER stress.

In some embodiments, the administration is effective to induce changes in N-glycan and O-glycan profiles. In embodiment, the administration is effective to induce reduction in Man6GlcNAc2 (Man-6), Man-8 and Man-9 in total oligomannose N-glycan content compared to an untreated control, accumulation of Man-5 and Man-7, and to decrease O-glycosylation following treatment with ManN.

Conversely, in some embodiments the present disclosure provides pharmaceutical compositions and methods for inhibiting angiogenesis, including but not limited to methods for treating malignant tumors and intraocular neovascular disorders in a subject, including administering to the subject in need thereof an effective amount of an inhibitor of hexosamine D-mannosamine (ManN) or reducing the amount of ManN available to the subject.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2nd ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20th ed., (Lippincott, Williams & Wilkins 2003), and Remington, The Science and Practice of Pharmacy, 22th ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a pharmaceutical composition, and/or a method that “comprises” a list of elements (e.g., components, features, or steps) is not necessarily limited to only those elements (or components or steps), but may include other elements (or components or steps) not expressly listed or inherent to the pharmaceutical composition and/or method.

It is understood that aspects and embodiments of the present disclosure described herein include “consisting of” and/or “consisting essentially of” aspects and embodiments. As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a pharmaceutical composition, and/or method that includes materials, steps, features, components, or elements, in addition to those literally disclosed, provided that these additional materials, steps, features, components, or elements do not materially affect the basic and novel characteristic(s) of the claimed subject matter. The term “consisting essentially of” occupies a middle ground between “comprising” and “consisting of”.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In some embodiments, “about” can be used to mean, for example, within 10% of the recited value, within 5% of the recited value, or within 2% of the recited value.

As used herein, “patient” or “subject” means a human or other mammalian subject to be treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopocia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing pharmaceutical compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the pharmaceutical compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses. The phrase “a therapeutically effective amount” also refers to an amount of an N-glycosylation inhibitor described herein that results in a measurable therapeutic response. A therapeutic response may be any response that a user of the method (e.g., a clinician) will recognize as an effective response to the therapy, including improvement of one or more symptoms (e.g., one or more symptoms of stroke or other ischemic disorder) and surrogate clinical markers (e.g., stroke biomarkers). A therapeutic response will generally be an amelioration or inhibition of one or more symptoms of a disease or condition, (e.g., ischemic stroke, hemorrhagic stroke, or an ischemic condition). Measurable therapeutic response also includes a finding that one or more symptoms of a disease or a disease or disorder is prevented or has a delayed onset, or is otherwise attenuated by a therapeutic agent described herein (e.g., an N-glycosylation inhibitor), thus, a “therapeutically effective amount” as used herein refers to an amount sufficient to reduce one or more symptom(s) or condition(s) associated with an ischemic stroke including but not limited to hemorrhagic transformation, disruption of the blood-brain barrier, increase in hemoglobin levels, and mortality.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In some embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In some embodiments, subjects with familial history of a disease are potential candidates for preventive regimens. In some embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety. The term “derivative” refers to a chemical substance related structurally to another substance, or a chemical substance that can be made from another substance (i.e., the substance it is derived from), e.g., through chemical or enzymatic modification.

The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent agent or compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66:1, 1977). In some embodiments, a “pharmaceutically acceptable salt” is intended to mean a salt of a free acid or base of an agent or compound represented herein that is non-toxic, biologically tolerable, or otherwise biologically suitable for administration to the subject. Sec, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. An agent or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosul fates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, [gamma]-hydroxybutyrates, glycolates, tartrates, and mandelates.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

As disclosed in the following examples, hexosamine mannosamine (2-Amino-2-deoxy-D-mannose or ManN hereafter) inhibits protein glycosylation and yet stimulates EC proliferation in vitro. The biological effects of ManN in other in vitro and in vivo models, as well as its possible mechanisms of action were investigated. ManN is an EC mitogen and survival factor for bovine and human microvascular EC, with an additivity with VEGF. ManN inhibits glycosylation in ECs and induces significant changes in N-glycan and O-glycan profiles. ManN and two N-glycosylation inhibitors stimulate EC proliferation via both JNK activation and the unfolded protein response caused by ER stress. ManN results in enhanced angiogenesis in a mouse skin injury model. ManN also promotes angiogenesis in a mouse hindlimb ischemia model, with accelerated limb blood flow recovery compared to controls. In addition, intraocular injection of ManN induces retinal neovascularization. Therefore, activation of stress pathways following inhibition of protein glycosylation can promote EC proliferation and angiogenesis and may represent a therapeutic strategy for treatment of ischemic disorders.

### Example 1

The effect of ManN on EC proliferation was assessed. A library of 619 highly purified metabolites encompassing a broad spectrum of chemical entities was screened for their ability to affect growth of bovine choroidal microvascular EC (BCEC), in the presence or in the absence of VEGF. This and similar assays have been previously employed to identify and characterize angiogenesis stimulators and inhibitors. Under the conditions tested, little or no proliferation was detected in the absence of VEGF.

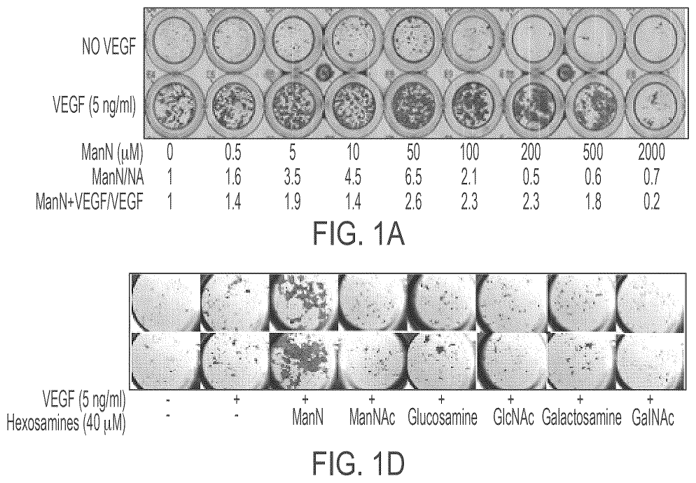

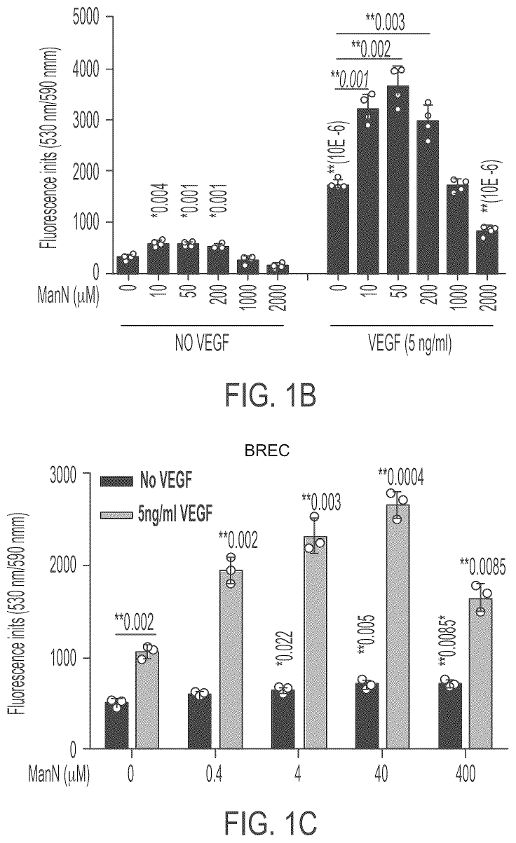

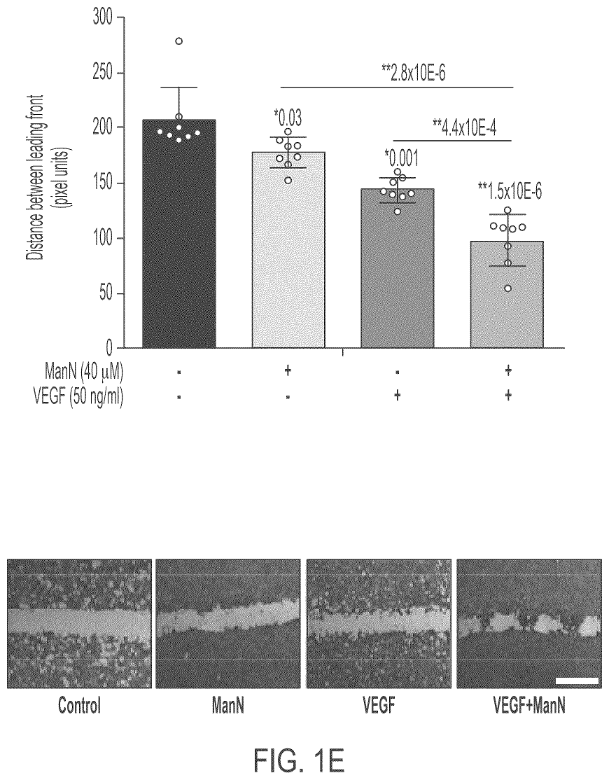

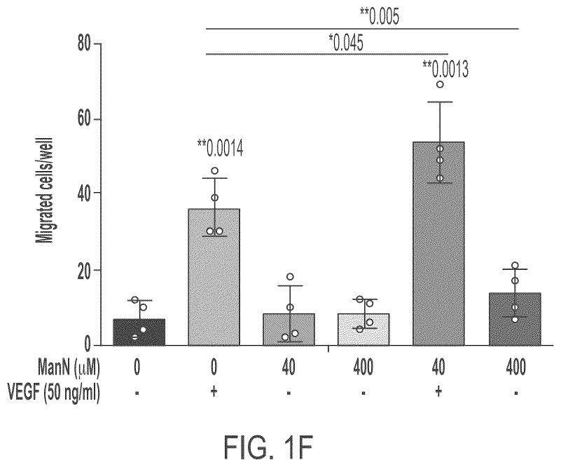

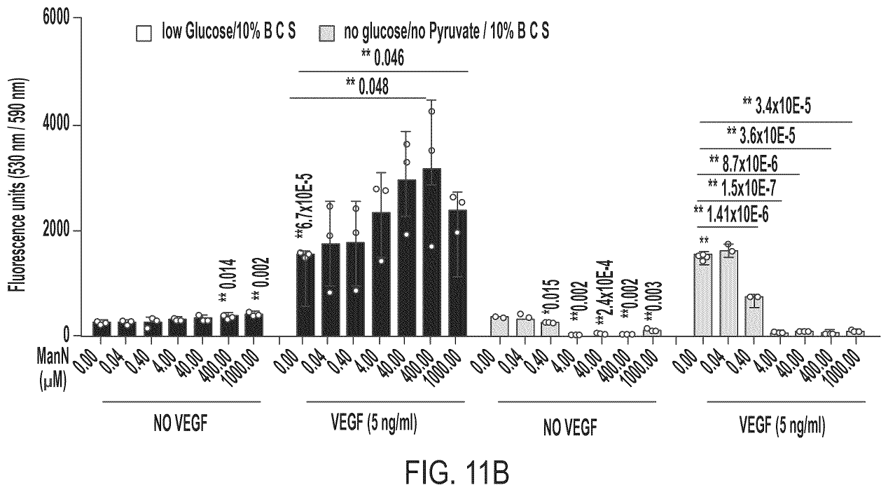

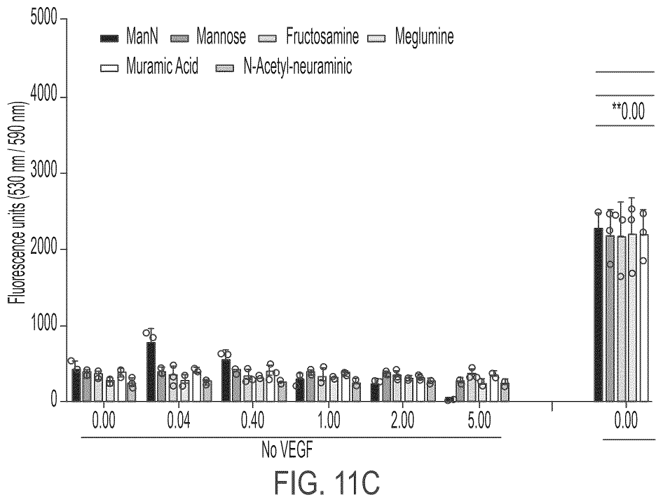

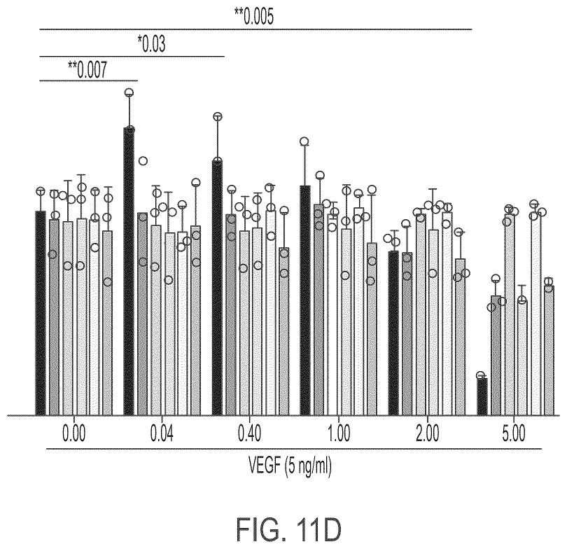

An initial screening was done, testing each compound at the concentrations of approximately 1 and 10 μM (assuming a molecular mass of 100 Da for each compound), with or without 5 ng/ml VEGF, which could induce ˜4 5 fold increase in cell proliferation. Six compounds of various chemical nature showed some inhibitory or stimulatory activity. The analysis was focused on one of these, ManN, a hexosamine originally identified as a component of bacterial cell wall because it showed the most potent and consistent effects. ManN had significant stimulatory effects in the 5-500 UM dose range and was also additive with VEGF in promoting BCEC proliferation. Dose-dependent effects of ManN on BCEC proliferation, in the absence or in the presence of VEGF, are shown in FIGS. 1A and 1B. A maximal ˜6.5 fold increase in EC-covered surface by ManN at 50 μM alone (FIG. 1A) or ˜2.5 3 fold increase in fluorescence units upon AlamarBlue addition (FIG. 1B) was obtained when cells were treated with 50 μM ManN and 5 ng/mL VEGF, compared to VEGF alone. AlamarBlue detects mitochondria activity as an indication of cell viability which correlates with cell number at certain ranges. The effects of ManN had a bell-shaped dose-response curve, with inhibition at higher concentrations (FIGS. 1A and 1B). Additive effects of ManN in promoting BCEC proliferation were observed also with bFGF (FIG. 11A) and in bovine retinal EC (BRECs) (FIGS. 11C and 11D).

Various hexosamines (galactosamine, glucosamine and their N-acetyl derivatives) were tested alongside ManN in the BCEC proliferation assay. However, none of these hexosamines had significant stimulatory effects (FIG. 1D). Several structurally related molecules such as D-isoglucosamine (fructosamine), meglumine, muramic acid, N-Acetylneuraminic acid (sialic acid present in all mammalian cells), glucose, and mannose were also tested. None of these molecules stimulated BCEC proliferation, with or without VEGF (FIGS. 11C and D).

ManN entered and accumulated inside the cells in a concentration-dependent manner. 0.66 nmol of ManN were detected in 1 mg cell lysate when BCECs were treated with

400 μM ManN for 2 hours. Following entry into the cells, ManN, but not mannose, is quickly converted to ManN 6 phosphate (ManN-6p). No incorporation of ManN was detected in N-glycans. Efficient uptake of ManNAc, and mannose has been reported.

The effects of ManN on BCEC proliferation were dependent on cellular glycolysis. The additivity between ManN and VEGF was abolished when glucose-free media was used. On the other hand, the activity of VEGF was not dependent on the glycolysis pathway (FIG. 11B). However, there was a significant cytotoxicity with as little as 4 μM ManN, even in the presence of VEGF, in glucose-free media.

To further characterize the effects of ManN on EC survival, proliferation and migration, confluent BCEC monolayers were mechanically wounded. FIG. 1E shows that 40 μM ManN or 50 ng/ml of VEGF significantly accelerated BCEC migration and/or proliferation as reflected by more complete closure of the “scratched” area, compared to the control group after 48 hours. Similar to the proliferation assays, additivity was observed when cells were treated with both ManN and VEGF (FIG. 1E). In addition, ManN at 40 μM showed a significant additivity with VEGF in promoting BCEC migration (FIG. 1F).

The observations were extended to human retinal microvascular EC (hRMECs), HUVEC and dermal microvascular endothelial cells (hDMVECs). ManN by itself stimulated HUVEC and hDMVECs growth. In addition, there was a dose-dependent additivity with VEGF in all EC types tested, with minimal toxicity even at 5 mM. Likewise, stimulation of migration and wound closure was observed in HUVEC treated with 40 μM ManN alone (migration assay) and/or in combination with 50 ng/ml VEGF (scratch assay).

### Example 2

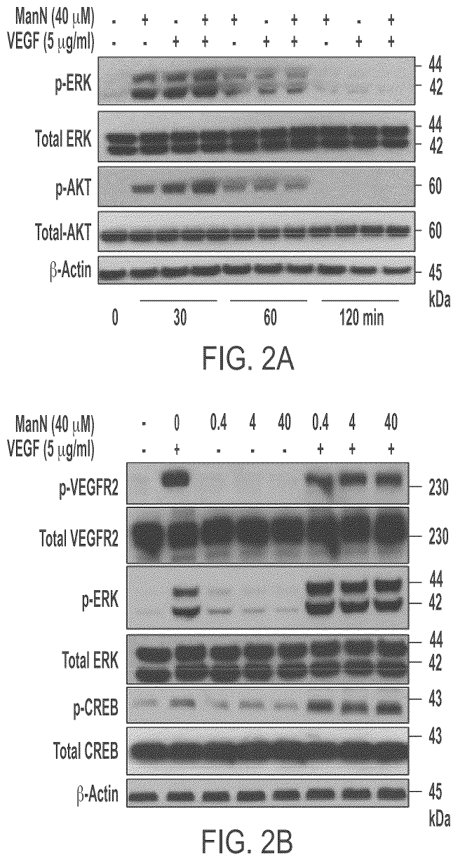

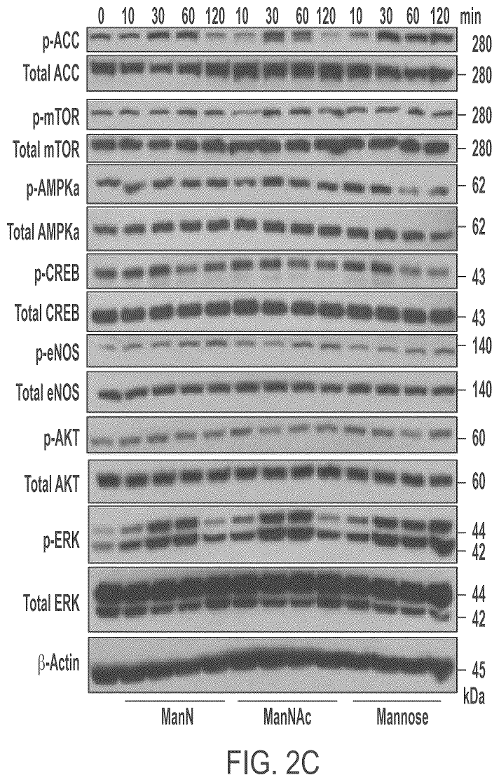

Activation of ERK, AKT, mTOR, CREB, AMPK, ACC and eNOS is not unique to ManN. It has been reported that cross-talk between signaling and metabolic pathways in the vasculature, such as insulin signaling and glucose metabolism in ECs, involves AKT and STAT3 activation. Together, they affect glycolysis, EC sprouting, proliferation and migration. The effect of ManN and/or VEGF on activating major signal transduction pathways known to promote proliferation, such as ERK, AKT, mTOR and CREB (CAMP response element binding protein) in BCECs was assessed. ManN activated ERK, AKT, mTOR and CREB at 40 μM. Stimulation of ERK, AKT and CREB was rapid and occurred within 10-30 minutes after adding ManN (FIGS. 2A to 2C). Further, an enhancement in activation of ERK, AKT and CREB was observed when both ManN and VEGF were present compared to ManN or VEGF alone (FIGS. 2A and 2B). The effect of ManN on activating the ACC (Acetyl-CoA carboxylase)/eNOS (endothelial nitric oxide synthase 3) pathway was assessed. Activation of the energy sensor AMPK (AMP-activated protein kinase) leads to cNOS activation and NO (nitric oxide) production; the latter exerts bell-shaped effects on EC proliferation. Both eNOS and ACC were significantly activated by 40 μM ManN within 10-30 minutes (FIG. 2D). However, activation of ERK, AKT, mTOR, CREB, ACC and eNOS was not unique to ManN. Indeed, other hexosamines such as ManNAc and mannose induced a similar activation of these signal transduction pathways (FIG. 2C). While activation of these common proliferation pathways likely contributed, without being bound by theory, some unique mechanism(s) may be implicated in the EC mitogenic effects of ManN.

### Example 3

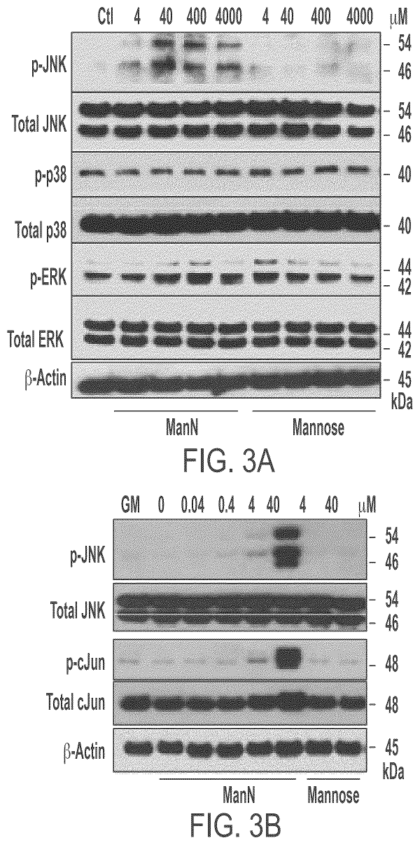

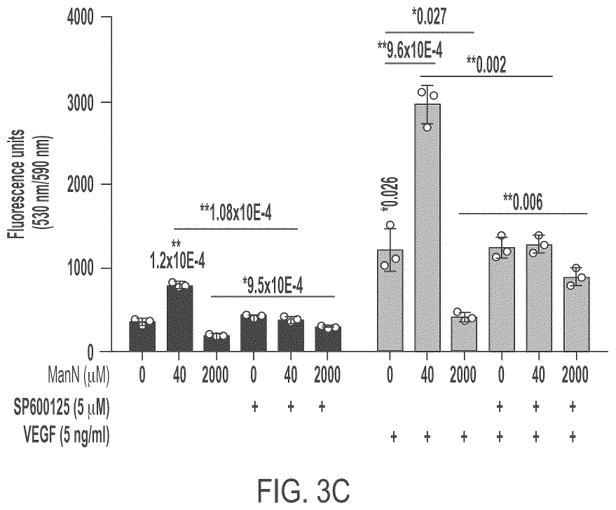

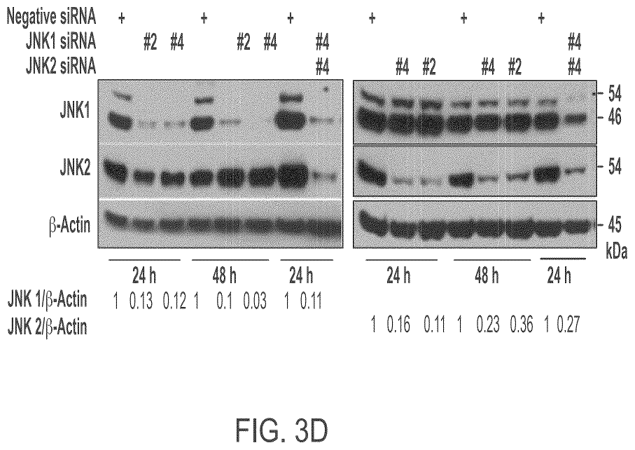

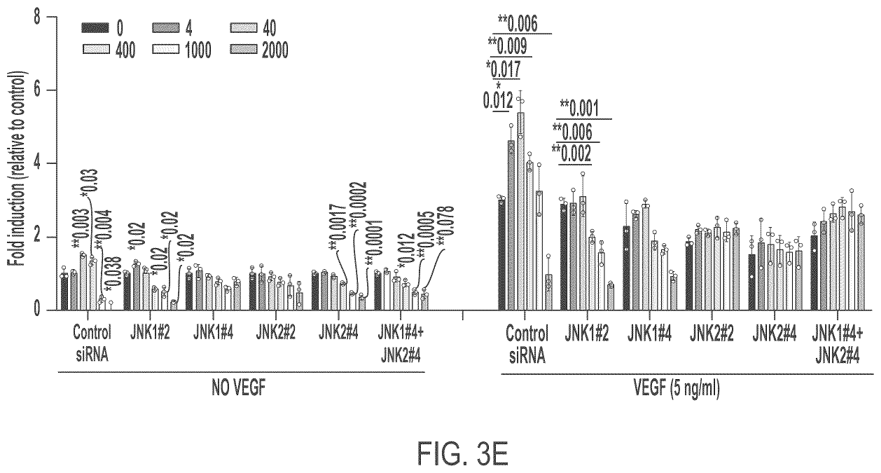

A series of specific pharmacological inhibitors was used to identify JNK/c-jun as a signal transduction pathway uniquely activated by ManN, among hexosamines. Western blot analysis revealed that, among three MAPK family members (ERK, p38, JNK), JNK was specifically activated by ManN. When growing BCECs were switched to proliferation assay media without growth factors, JNK and its downstream c-Jun were significantly activated by ManN in a dose-dependent manner, but not by mannose (FIGS. 3A and 3B). ManN, but not other hexosamines tested, activated the JNK pathway. Treatment of BCECs with the JNK specific inhibitor SP600125 (5 μM) abolished the effects of ManN on BCEC proliferation (FIG. 3C).

The effects of ManN on BCECs were assessed following transfection with siRNA against JNKs (namely JNK1 and JNK2, as JNK3 is not expressed in BCECs). Knocking down-80% of either JNK1 and/or JNK2 by two independent siRNAs against JNK1 or JNK2 abolished mitogenic effects of ManN on BCECs at μM concentrations (FIGS. 3D and 3E), indicating that both JNK1 and JNK2 are important in transducing stress signals.

### Example 4

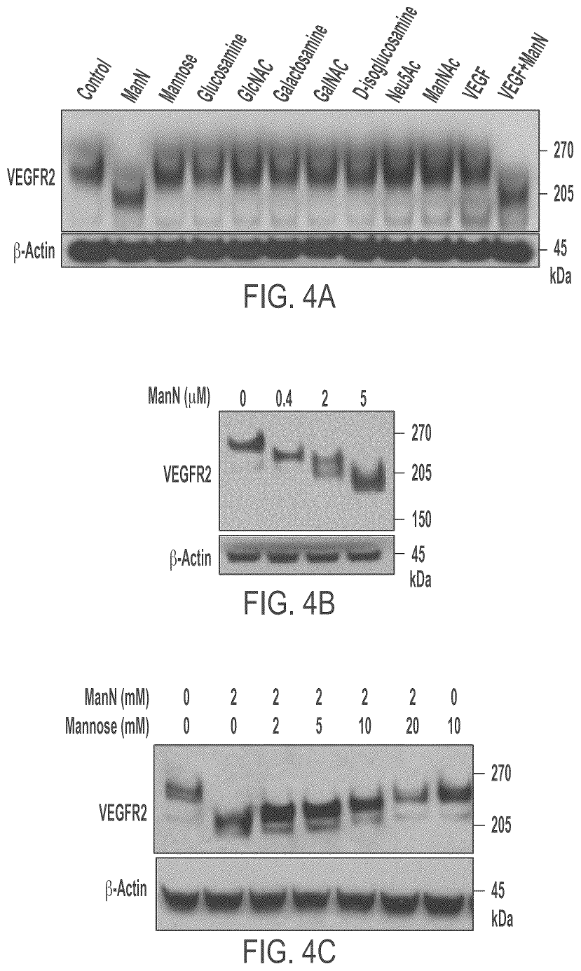

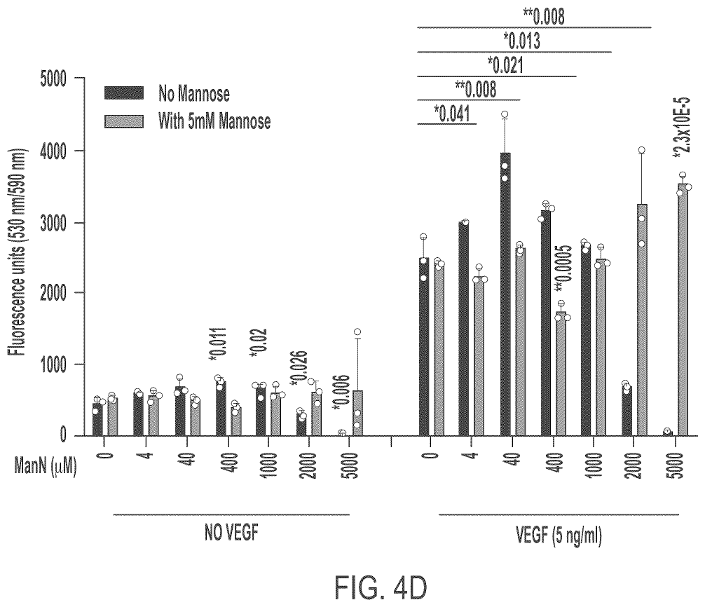

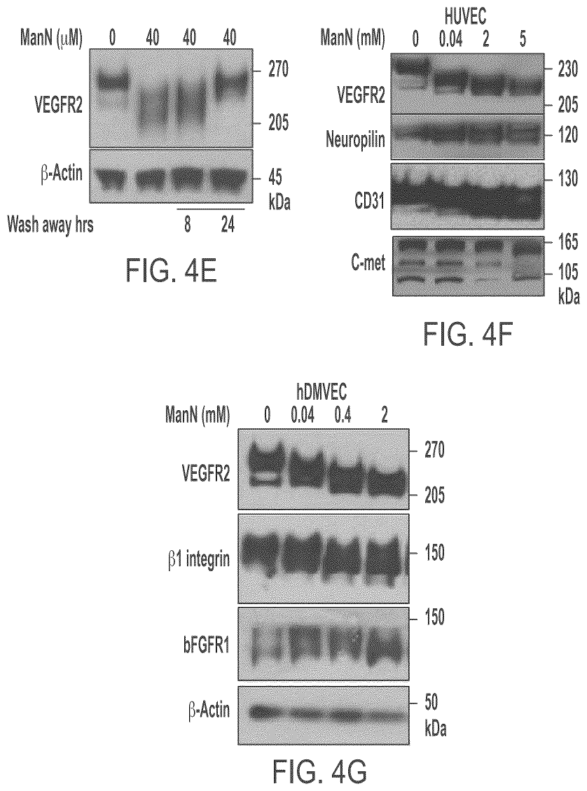

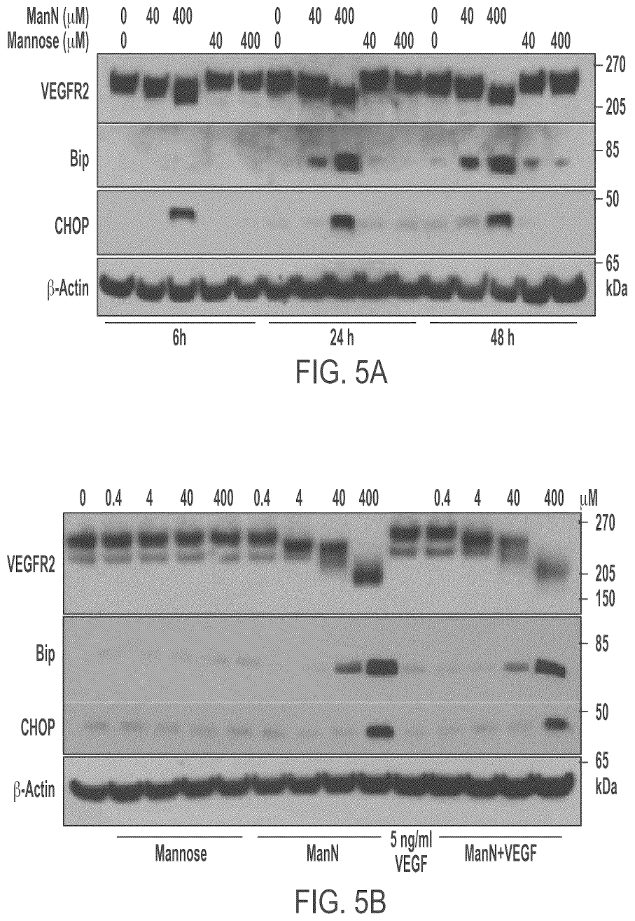

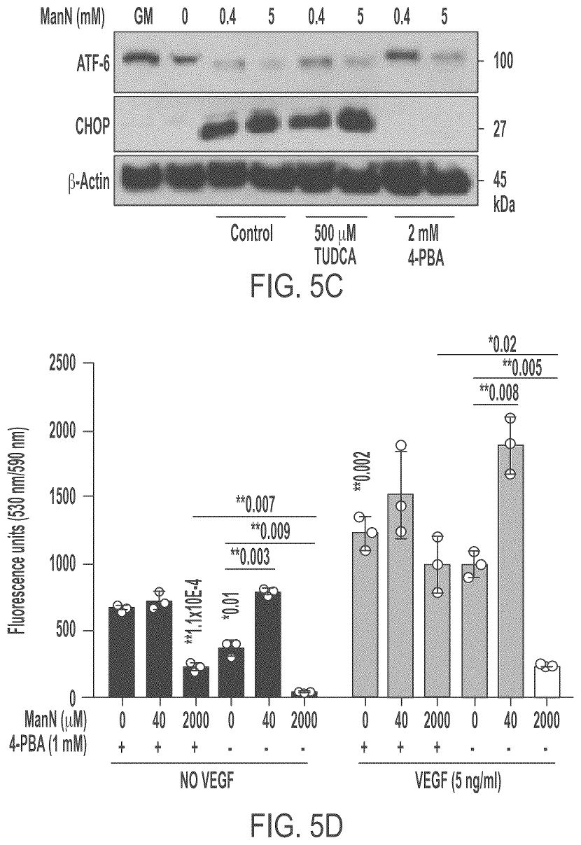

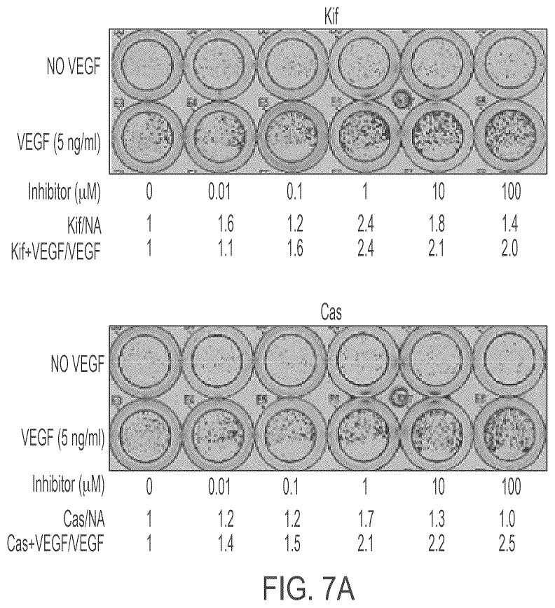

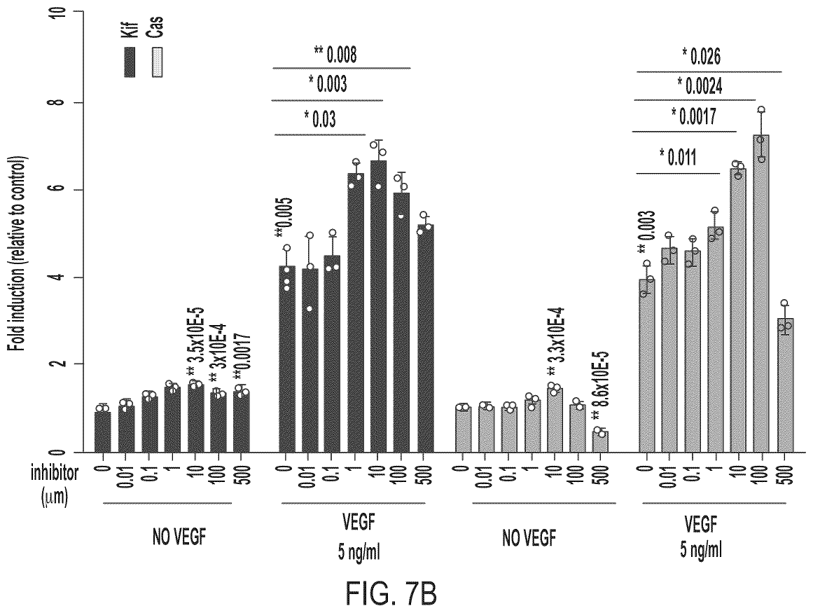

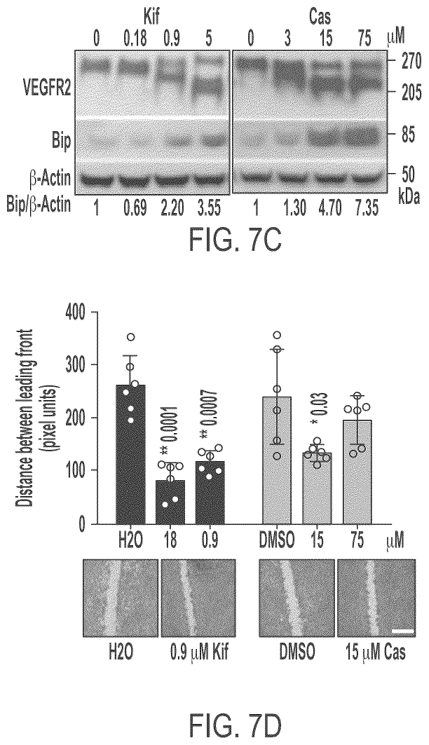

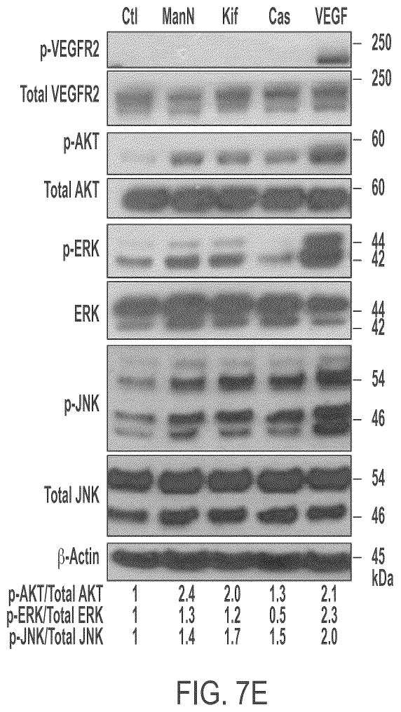

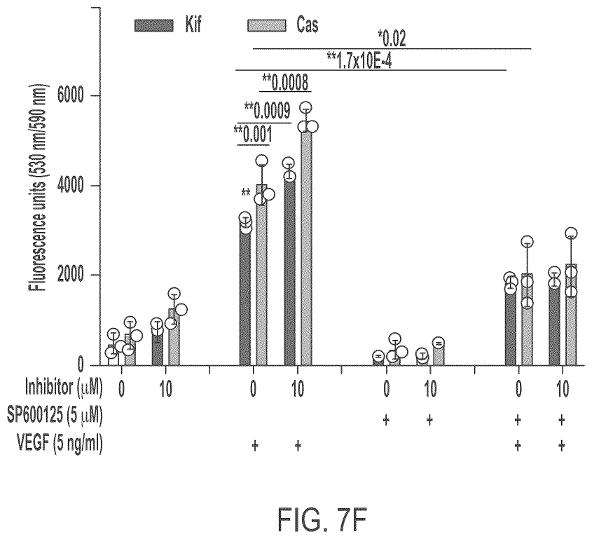

ManN affects protein glycosylation in endothelial cells. The additivity of ManN with VEGF could potentially occur at transcriptional and/or translational level or through signal transduction pathways mediated by VEGF-VEGFR2. However, neither transcription of VEGF, VEGFR2 and GLUT] & 4 nor total VEGFR2 protein expression was significantly changed in BCECs (FIGS. 4A to 4C; FIGS. 5A and 5B; FIGS. 7C and 7E) when cells were treated with various concentrations of ManN for 4 hours (for gene expression level) or 24 hours (for protein expression level). The same was true for BRECs and hRMVECs. Biotinylation studies showed no changes in the amount of VEGFR2 on cell surface. However, VEGFR2 phosphorylation in response to VEGF was decreased in ManN pre-treated cells, suggesting that VEGFR2 activation was hampered, rather than enhanced in BCECs. No ligand-independent VEGFR2 activation occurred after ManN addition in BCECs. The same was true also for HUVECs (SFIG. 10a) and hDMVECs.

The apparent molecular mass of VEGFR2 shifted significantly following ManN treatment in both BCECs (FIGS. 4A to 4C and 4E) and BRECs, starting at 40 μM. New lower molecular weight bands (−170-200 KDa) appeared in BCECs treated with ManN in a dose-dependent manner, compared to the control (a major band at −230 KDa and a minor band at −210 KDa) (FIGS. 4B, C, and E; FIGS. 5A and 5B; and FIG. 7C). This shift was unique to ManN among hexosamines and their derivatives (FIG. 4A). VEGF alone had no effect on molecular mass. Adding VEGF to ManN caused no additional shifts (FIG. 4A). Lower molecular weight VEGFR2 bands are not likely degradation products since ManN removal completely reverse the effects of ManN on molecular mass after 24 hours (FIG. 4E). However, based on PNGase F treatment, it appears that not all the glycosylation on VEGFR2 was abolished by ManN, at least at μM concentrations. Experiments with the small molecule tyrosine kinase inhibitor axitinib, a potent VEGFR2 inhibitor, indicate that decreases in VEGFR2 molecular mass and stimulation of BCEC proliferation by ManN are not dependent on VEGFR2 signaling.

A significant change in VEGFR2 protein mass was also observed in hRMVEC, HUVEC (FIG. 4F) and hDMVECs (FIG. 4G) with 40 μM ManN, whereas the additive effect of ManN and VEGF on proliferation of these cells occurred at mM levels.

To better understand how ManN may affect VEGFR2 post translational modification, cells were treated with ManN in the presence of one of four monosaccharides (mannose, glucose, galactose, or fucose) at a maximum 1:10 molar ratio. These monosaccharides are known to be important in protein N-glycosylation. Our results suggest that mannose could dose-dependently block ManN effects on VEGFR2 molecular mass as well as on BCEC proliferation (FIGS. 4C and 4D). The effects of mannose may not be limited to prevention of entry of ManN into cells via the same transporter(s) since the effects were seen when BCECs were first treated with ManN for 2 hours to ensure its successful cellular uptake. Glucose, but not galactose or fucose, had similar effects as mannose.

Decreases in protein mass following ManN administration in BCEC were not limited to VEGFR2. Other N-glycosylated growth factor receptors/co-receptors or adhesion molecules, including av integrin, Neuropilin-1, VE-cadherin and bFGFR1 were affected as well.

### Example 5

The effect of ManN on general protein glycosylation profile was assessed. N-glycosylation is a complex process, dependent on multiple enzymes that act sequentially on glycoproteins to generate hybrid and high-mannose glycan structures as they transit through the secretory pathway, from ER to Golgi apparatus. It plays an important role in the determination of the fate of newly synthesized glycoproteins in the ER, their correct folding, cellular destination and proper function.

Several key enzymes involved in protein N-glycosylation in both ER and Golgi apparatus were evaluated. α-mannosidase from Jack Bean is a broad-specificity exoglycosidase that catalyzes the hydrolysis of terminal, non-reducing α1-2, α1-3, and α1-6 linked mannose residues from oligosaccharides in both organelles, and controls conversion of high mannose to complex N-glycans, the final hydrolytic step in the N-glycan maturation pathway. This enzyme has been used to screen for potential N-glycosylation inhibitors. ManN, but not other hexosamines or their derivatives, showed inhibitory activity at 400 μM, which is considerably higher than the effective mitogenic concentrations in BCECs. No effect of ManN up to 2 mM on α- or β-glucosidases was detected.

N-linked glycans from BCECs were isolated by enzymatic cleavage, followed by purification and characterization using MALDI-TOF-MS. Treatment with 40 μM ManN resulted in a significant time-dependent reduction in Man6GlcNAc2 (Man-6), Man-8 and Man-9 in total oligomannose N-glycan content compared to the untreated control, whereas a significant early phase accumulation of Man-5 and Man-7 was observed after ManN treatment. ManN has been previously shown to inhibit Lipid-linked oligosaccharide (LLO) synthesis, to change protein GPI biosynthesis and hybrid glycan production as well as to incorporate into the glycans in MDCK cells. Accumulation of Man-5 over time suggested that inhibition of mannosidase is unlikely the mechanism of pro-angiogenic activity in BCECs.

The monosaccharide content was measured to profile the composition of complex N-glycans. A significant decrease in fucose (8 hours), mannose (12 hours), galactose (24 hours) and Neu5Ac (8 hours and 24 hours) were found in ManN-treated cells compared to the untreated control cells, consistent with the inhibitory activity of ManN on overall protein N-glycosylation.

O-glycan modification is another form of post translational modification of proteins, where a serine or threonine residue is covalently linked with a GalNAc residue. The GalNAc residue can be further modified by several glycosyl-transferases acting in a sequential manner to extend the glycan chain, either branched or linearly, according to substrate specificity. The ppGalNAcT (polypeptidyl GalNAc transferase) catalyzes the transfer of a a-GalNAc from UDP-GalNAc to Ser or Thr residue of a glycoprotein, producing the Tn antigen. When the Tn antigen is generated, it can have three different fates: (i) it can be sialylated on C6 by the enzyme ST6GalNAcT; (ii) it can be substituted on C3 or C6 by a f3-GlcNAc which gives rise to core-3 or core-6, respectively; or (iii) it can be galactosylated on C3 by the Cl GalTI in order to form core-1 which can also be sialylated to produce mono- or di-sialyl Core-1 O-glycan.

O-glycan analysis was conducted in BCEC lysates by MALDI-Tof mass spectrometry. Due to unavailability of a unique enzyme that cleaves all different forms of O-glycan, a reductive beta-elimination was performed to have an understanding of the O-glycan backbone. To protect from de-sialylation during mass spectral data acquisition, permethylation was performed prior to MALDI-Tof/Tof mass analysis. An overall decrease in O-glycosylation following treatment with 40 μM ManN. In particular, we observed a trend of decrease toward ion intensity at m/z of 895 (Sialyl-Corel, Galf31-3GalNAc-), 1256 (di-sialylated Core 1), 983 [Core 2, GlcNAcf31-6 (Galf31-3)-GalNAc-] and 1187 (di-galactosylated Core 2).

### Example 6

The effect of ManN on activating UPR by increasing Bip and CHOP expression was assessed. Asparagine linked N-glycosylation is one of the most common modification reactions in eukaryotic cells, occurring in proteins that are co-translationally translocated across or integrated into the ER during biosynthesis. After N-linked oligosaccharides are transferred to nascent proteins by the OST (oligosaccharyltransferase), ER resident glucosidases and mannosidases generate a series of glycan-trimming intermediates that are specifically recognized by ER-localized lectins to direct the nascent proteins into protein folding, degradation or export pathways. One of the consequences of inhibition of protein glycosylation is compromised protein folding, leading to ER stress. The physiological responses to the UPR are mediated by changes in gene expression, such as the regulation of ER Hsp70 chaperone BiP (also called glucose-regulated protein 78, binding of immunoglobulin protein) and another multifunctional transcription factor CHOP (CCAAT-enhancer-binding protein homologous protein). Impaired UPR function, for instance during aging, creates a permissive environment for protein aggregation, unresolved ER stress, and chronic inflammation.

To investigate a possible ManN-mediated ER stress, we studied the expression of Bip and CHOP in ManN or mannose-treated cells by western blot analysis. Our data indicate that ManN, but not mannose or VEGF, can significantly turn on Bip expression in a concentration-dependent manner when growing cells are deprived of growth factor supply, with accumulation of Bip being evident at 24 hours (FIGS. 5A and 5B) and 48 hours (FIG. 5A). CHOP induction appeared to be faster, at about 6 hours in a dose-dependent manner (FIG. 5A). No synergy between ManN and VEGF in promoting Bip or CHOP expression was noted (FIG. 5B).

We tested two well-known chemical chaperons 4-PBA (4-phenylbutyric acid) and TUDCA (tauroursodeoxycholic acid) to alleviate ER stress in ManN-treated BCECs. Both were previously shown to mitigate Tunicamycin-induced cIF2a-ATF4-CHOP arm of UPR and Bip expression. We found that 2 mM 4-PBA, but not 500 M TUDCA, could prevent the induction of CHOP expression by ManN at 400 μM and 5 mM and restore the expression of ATF-6 (Activating Transcription Factor-6) by ManN at 400 μM (FIG. 5C). Likewise, restoration of ATF-6 expression was much weaker by TUDCA compared to 4-PBA. As a transmembrane ER glycoprotein, ATF-6 is cleaved liberating a 50 kDa amino-terminal fragment that translocates to the nucleus which activates transcription of ER chaperones and ER-associated degradation components such as Bip and CHOP upon accumulation of improperly folded proteins in the ER. Pre-treating cells with 1 mM 4-PBA for 4 hours could effectively reverse the bell-shaped activity of ManN on BCEC proliferation in the absence or presence of VEGF. Additivity between ManN and VEGF was largely abolished (FIG. 5D).

### Example 7

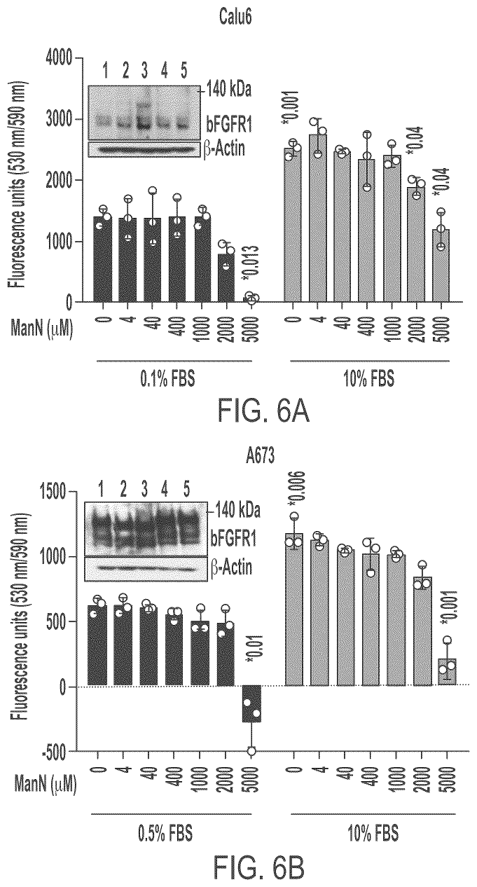

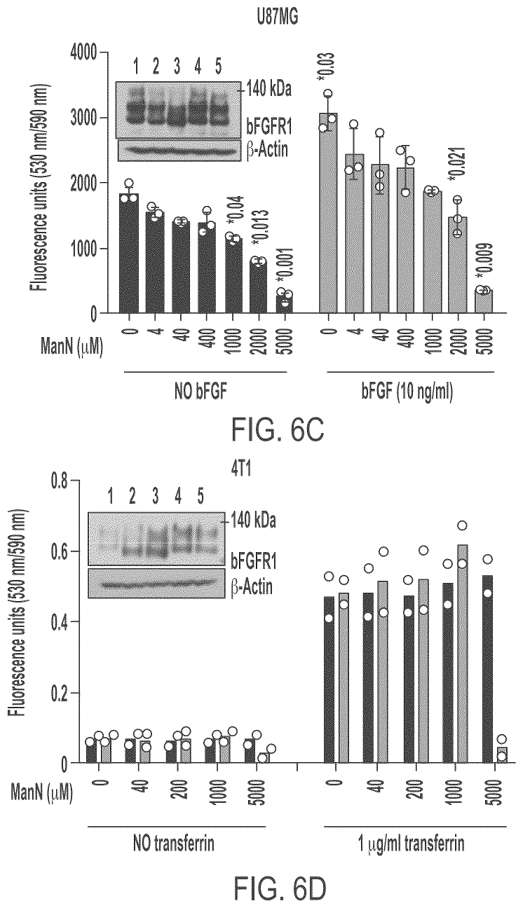

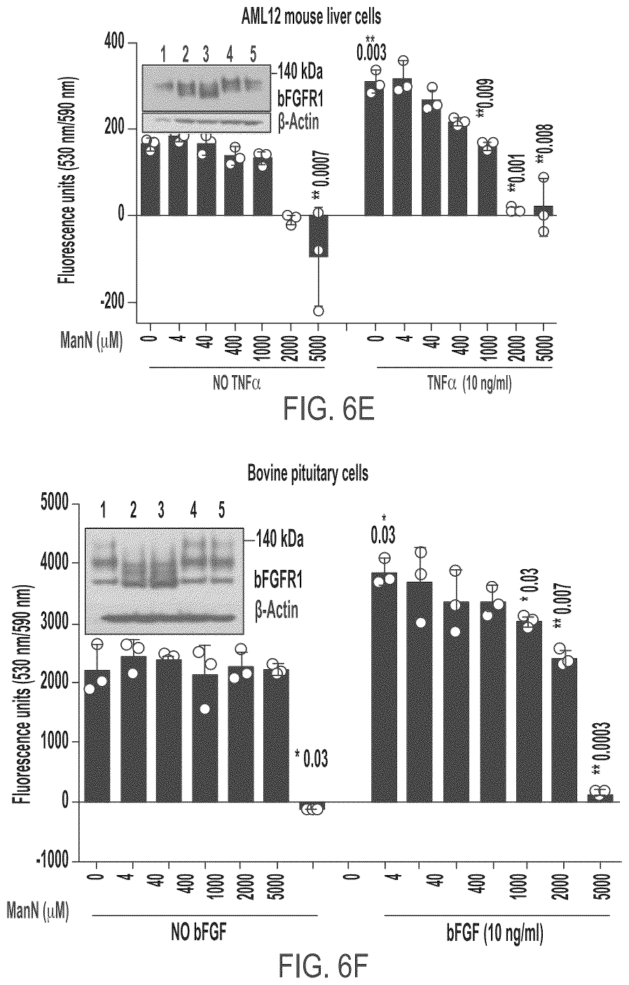

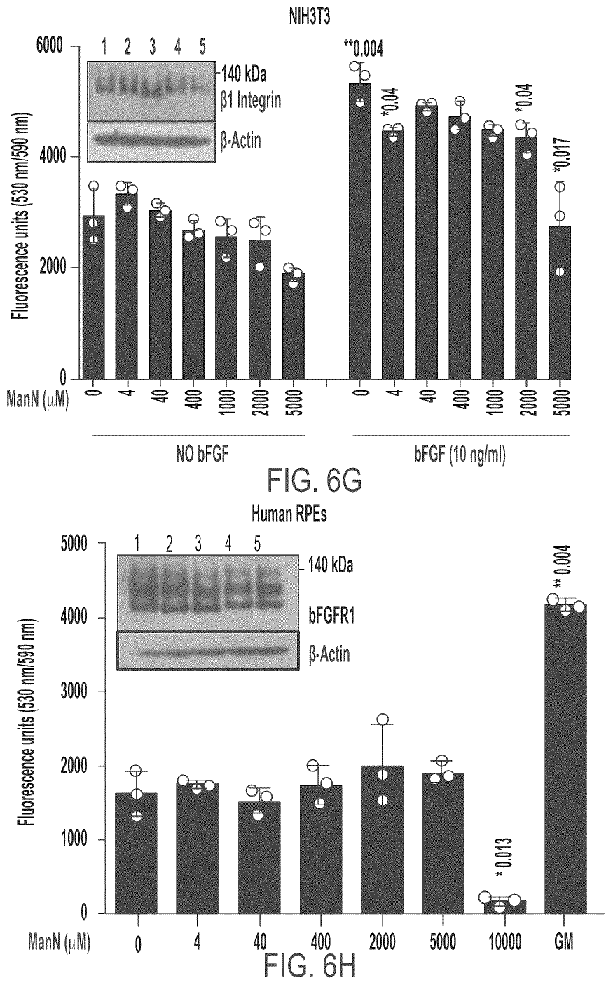

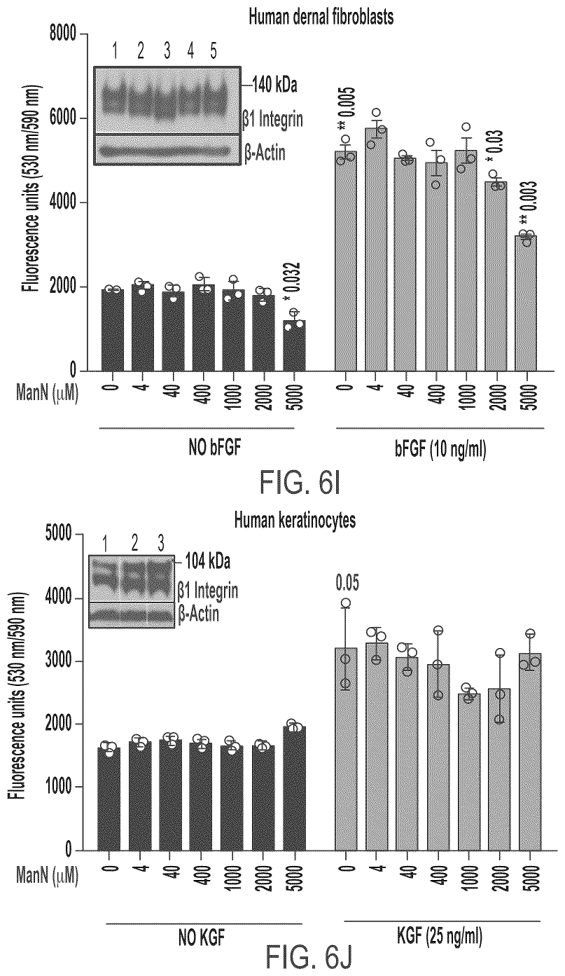

Effects of ManN on non-endothelial cells were assessed. To extend our observations in EC, we examined a variety of non-EC types from different species. These include NIH3T3 fibroblasts and AML12 liver cells (mouse), ARPE-19 RPE cells (human) and freshly isolated bovine pituitary cells. We also tested several human cell types related to our in vivo models such as dermal fibroblasts and keratinocytes. In addition, we screened four human or mouse cancer cell lines (A673, U87MG, Calu6 and 4T1) (FIG. 6). To examine post-translational modifications of proteins in non-ECs, we used bFGFR 1 or f31 integrin to monitor molecular mass change. Similar to BCECs, ManN not mannose could induce molecular mass change in all these non-ECs (FIG. 6 inserts). However, unlike BCECs (FIG. 1B), BRECs (FIG. 1C), hRMVECs, HUVECs and hDMVECs, no proliferative effects by ManN were observed at μM to mM concentrations, alone or in combination with other growth stimulators (FIG. 6), although efficient ManN uptake and comparable levels of free ManN were detected in all cell types. Cellular toxicity varied among different cell types, with AML12 being the most sensitive one and human RPE cells and human keratinocytes being the least sensitive ones to 5 mM level of ManN (FIGS. 6E and 6H). Growth inhibition by 25 mM Mannose in vitro has been reported in several tumor lines with low level of PMI (phosphomannose isomerase). At 5 mM, ManN, but not mannose, showed significant toxicity on 4T1 cells (FIG. 6D), possibly due to a higher PMI level in 4T1 relative to all the reported sensitive tumor lines.

### Example 8

Similar to ManN, inhibitors of protein N-glycosylation stimulate EC growth. To determine whether broad changes in protein glycosylation could promote cell proliferation, two well-characterized inhibitors, Kifunensine (Kif) and Castanospermine (Cas) were tested. BCEC proliferation was stimulated in a dose-dependent fashion in the absence or in the presence of 5 ng/ml VEGF (FIGS. 7A and 7B). Following treatment with Kif or Cas for 24 hours, reduction in VEGFR2 molecular mass on SDS-PAGE was evident (FIG. 7C).

At 40 μM, Kif could significantly activate ERK and AKT in BCECs (FIG. 7E), HUVEC and hDMVECs. Activation of ERK by Cas was less obvious in both BCECs and hDMVECs. However, both inhibitors were able to activate the JNK pathway in BCECs (FIG. 7E). Blocking JNK activation with 5 μM SP 600125 significantly reduced the effects of both glycosylation inhibitors on proliferation of BCECs (FIG. 7F). FIG. 7C illustrates a dose-dependent induction of Bip expression when growing BCECs were switched to media without growth factors for 24 hours in the presence of Kif or Cas at concentrations which promoted cell proliferation.

Both Kif and Cas had significant activity in the BCEC “scratch” assay, with gaps being closed more rapidly by each molecules over 48 hours relative to control (FIG. 7D). The inserted panel of FIG. 7D shows representative images from an assay in which Kif or Cas was used. Quantification analysis indicated that there was significant acceleration of gap closing in a dose-dependent manner compared to controls.

### Example 9

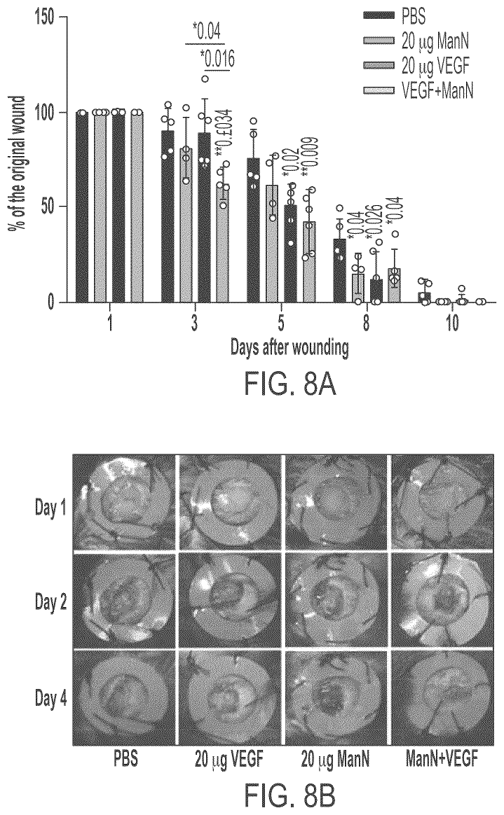

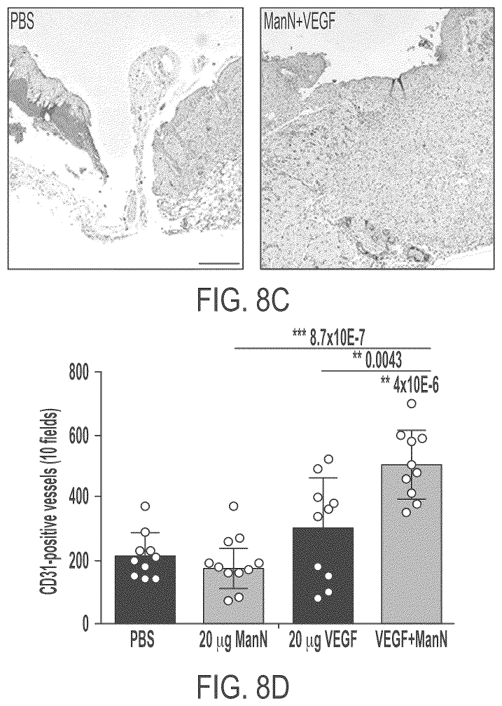

The relation between effects of ManN on endothelial cells in vitro and in angiogenesis in vivo, was investigated via its effects in a splinted wound model in mice. In this model, the repair process is entirely dependent on epithelialization, cellular proliferation and angiogenesis, which closely mirror the biological processes of human wound healing. The effects of ManN and VEGF were tested alone or in combination. Topical applications of 20 μg of VEGF or 20 μg of ManN daily was done for the first 3 days after wounding. When VEGF and ManN were combined, a significant acceleration of wound closure was observed during the early phase of healing (FIG. 8A). Compared with VEGF or ManN monotherapy, the combination had a significant faster wound closure starting from day 2 (FIG. 8B). On day 4, an average closure of the wound was 81.5%, 75.6%, 66.9% and 29.8% in PBS-, ManN-, VEGF- and combination-treated group, respectively. Small vessel numbers were quantified around the wound area at day 4. A significant increase in CD31-positive vessels was found in the combination group compared to PBS control, VEGF or ManN alone (FIGS. 8C and 8D).

Thus, ManN, in combination with VEGF, promotes angiogenesis in a skin injury model. In this acute model, wound closure takes place rapidly, without any treatment.

The stability of ManN was assessed in wound fluid contaminated by bacteria, a common feature of wounds. ManN was added to freshly collected wound fluid from a mouse model of skin infection with Staphylococcus aureus, a prevalent cause of skin and soft tissue infections in humans. No significant loss of free ManN was detected following incubation with such wound fluid for up to 24 hours at 37° C. Thus, ManN may be useful for treatment of infected wounds, possibly in combination with anti-microbials or other agents.

One of the known properties of VEGF is a rapid induction of vascular permeability following injection in the guinea pig skin. The effect of ManN in inducing vascular permeability was assessed in the same assay. However, no permeability-enhancing effects were elicited by ManN when tested at 1 ng-5 μg, while 25 ng VEGF induced vascular permeability.

### Example 10

The angiogenic effects of ManN and Kif in a mouse hindlimb ischemia model were assessed. The activity of ManN in a chronic ischemia model that might more specifically reflect its effects as an endothelial cell mitogen and a pro-angiogenic factor was assessed, and the hindlimb ischemia model in the mouse was considered. Several variants have been described, depending on which vessel is occluded. The variant chosen consists in ligation and excision of the femoral artery, which results in more severe ischemia compared to simple femoral artery ligation. Occlusion of two vessels produces more severe ischemia, but has the disadvantage of inducing severe pain and distress, as well as frequent ulcerations and necrosis in mice.

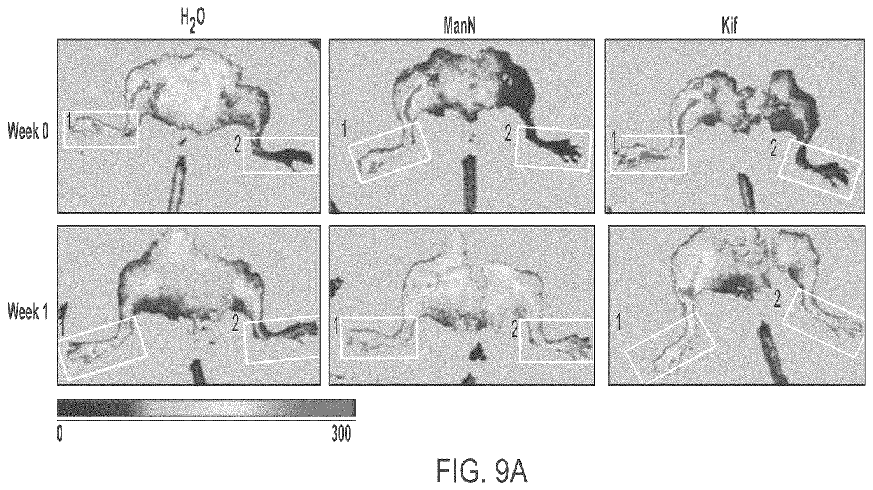

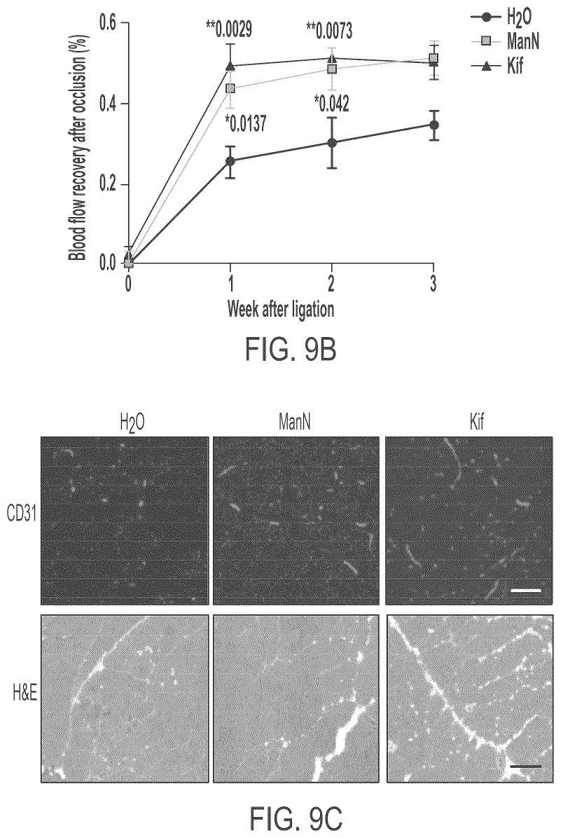

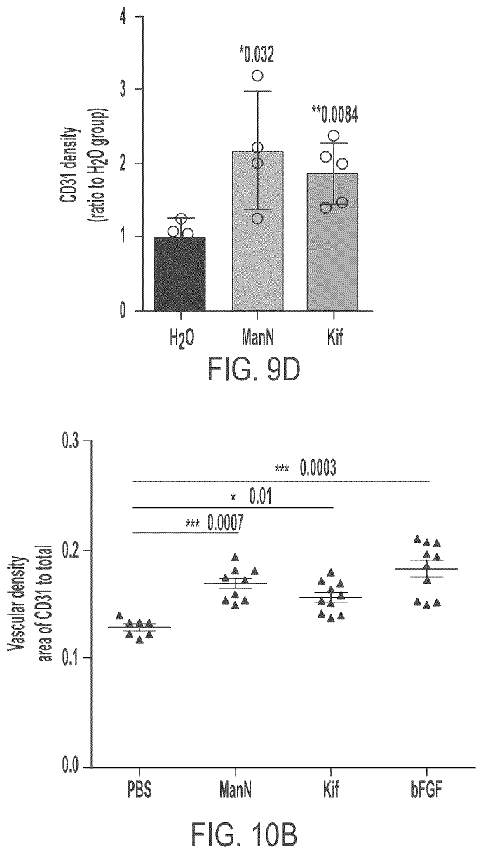

Oral administration of ManN was tested in this femoral artery ligation-excision model. Since Kif has been previously administered intraperitoneally for in vivo studies, this route was employed. Laser Doppler Perfusion Imaging (LDPI) was used as a non-invasive method to monitor time and extent of the blood flow recovery in the ischemic limb. Serial examination of blood flow was taken with LDPI and increment of the perfusion ratio of ischemic (ligated; left side) to non-ischemic (sham; right side) hindlimbs after ligation was used to indicate a recovery of blood flow. Starting immediately after surgery, mice were orally fed with 20% ManN or 1 mg/ml Kif ip every other day, as described in methods. One week after surgery, the perfusion ratio in H2O-fed group indicated a blood flow recovery of −25%, a value that is in good agreement with published data with the same type of lesion, in the same strain of mice. However, the blood flow recovery in ManN and Kif-treated group was about 40% and 47%, respectively, which demonstrated an accelerated recovery rate of blood flow compared to H2O-treated mice (FIGS. 9A and 9B). The blood perfusion ratio continued increasing to −50% of sham-treatment limbs in 3 weeks after ManN and Kif treatment and was significantly higher than the control group (FIGS. 9A and 9B).

Consistent with the improved blood flow, the ischemic hindlimbs of ManN-treated and Kif-treated group showed an increased blood vessel density compared to the control group, as assessed by CD31 immunostaining of the surrounding muscle tissue 3 weeks post-ligation. Compared with H2O-treated control group, blood vessel densities were respectively 2.3 and 1.8 times higher in ManN and Kif-treated groups (FIGS. 9C and 9D).

Following oral administration, there was a relatively rapid decline in ManN plasma levels. Plasma free ManN levels reached a peak level of −100 nmol/ml plasma at 1 hr. After 3 hours, only about half of that amount was detectable. 2 hours after oral feeding of 20% ManN, muscle samples were taken from the ischemic legs. A significant amount of ManN reached the ischemic legs, with 0.17+1−0.18 nmol/mg protein of free ManN and 0.91+1−0.24 nmol/mg protein of ManN-6p. At least in BCECs, ManN effects on protein mass lasted for at least 8 hours in the absence of exogenous ManN (FIG. 4E), indicating that even a relatively brief exposure may be adequate to elicit pharmacological effects.

### Example 11

The effect of ManN and Kif on inducing retinal neovascularization was assessed. The findings in cultured eye-derived EC were extended to a suitable in vivo model system. The mouse retina has been used extensively over the past decades to study both physiological and pathological angiogenesis. To obtain a detailed description of the retinal vasculature, images from retinal flat mounts were processed for vascular area fraction (ratio of area covered by blood vessels to total retinal area). Using this model, the effects of ManN in retinal neovascularization were assessed. Kif was also tested in this model because it is a water-soluble inhibitor and its mechanism of glycosylation inhibition is well established. In addition, it shares with ManN the ability to activate ERK, AKT and stress pathways in BCEC (FIG. 7E).

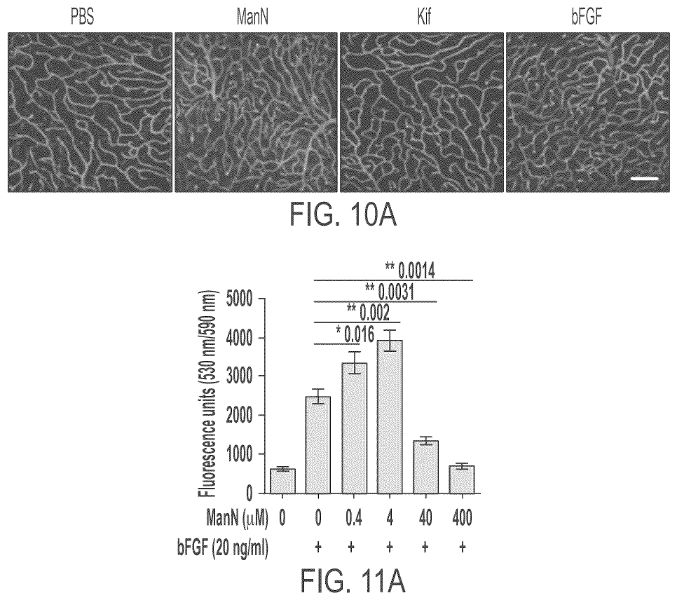

Five hundred nanograms of ManN or Kif was intravitreally injected and the retinal vasculature was examined after seven days. Intravitreally administration of 200 ng bFGF was used as a positive control in this model. In bFGF, ManN and Kif-treated group, the density of retinal vessels was increased by about 35%, 30% and 20%, respectively, compared to PBS group (FIGS. 10A and 10B).

### Example 12

The effects of ManN treatment on brain infarction (induced via MCAO) were tested. The experiments were conducted in a model of acute ischemia-reperfusion-induced brain injury in the mouse and represent the combined use of pretreatment and post-treatment of an N-glycosylation inhibitor. In this instance, ManN was tested as the inhibitor of N-glycosylation. The MCAO model in the mouse is a well-studied and reproducible example of an acute ischemic event to a single hemisphere of the brain. In contrast to other in vivo ischemia models disclosed herein (e.g. the hindlimb ischemia model and evaluation of blood flow), the MCAO model has an acute phase of ischemia (2 hours) after which time normal blood flow is restored to the affected region. In this instance, effects of ManN treatment were evaluated on a tissue particularly sensitive to brief disruptions of blood flow (e.g. brain tissue) and assayed at time points after normative blood flow had already been restored.

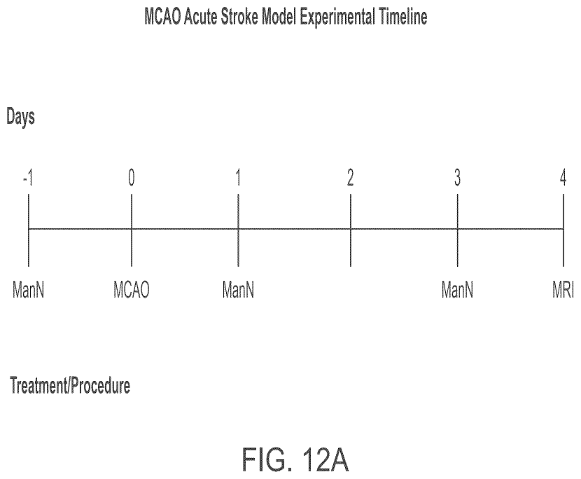

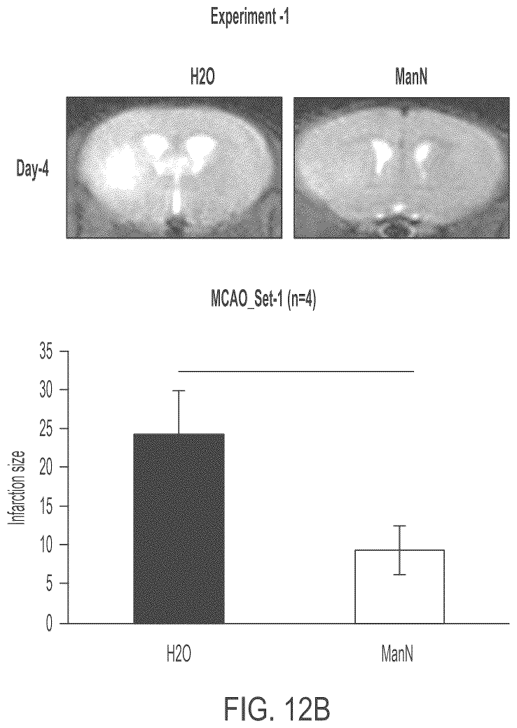

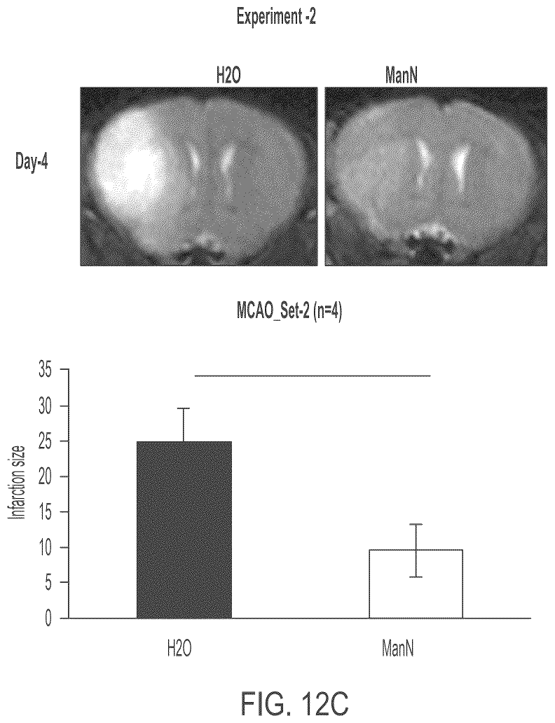

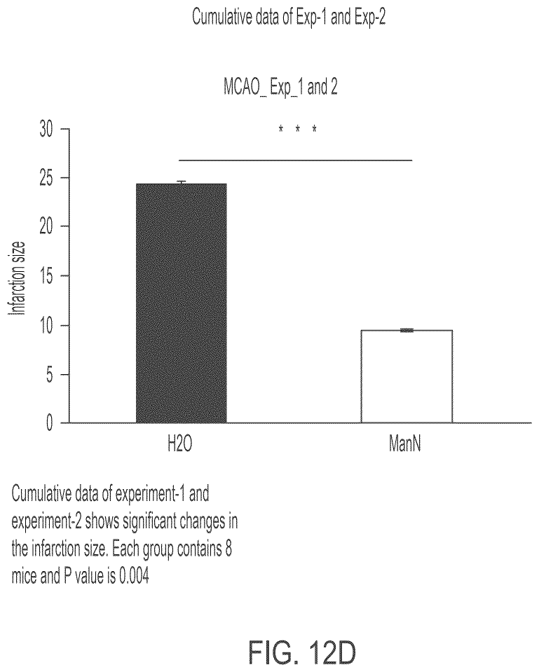

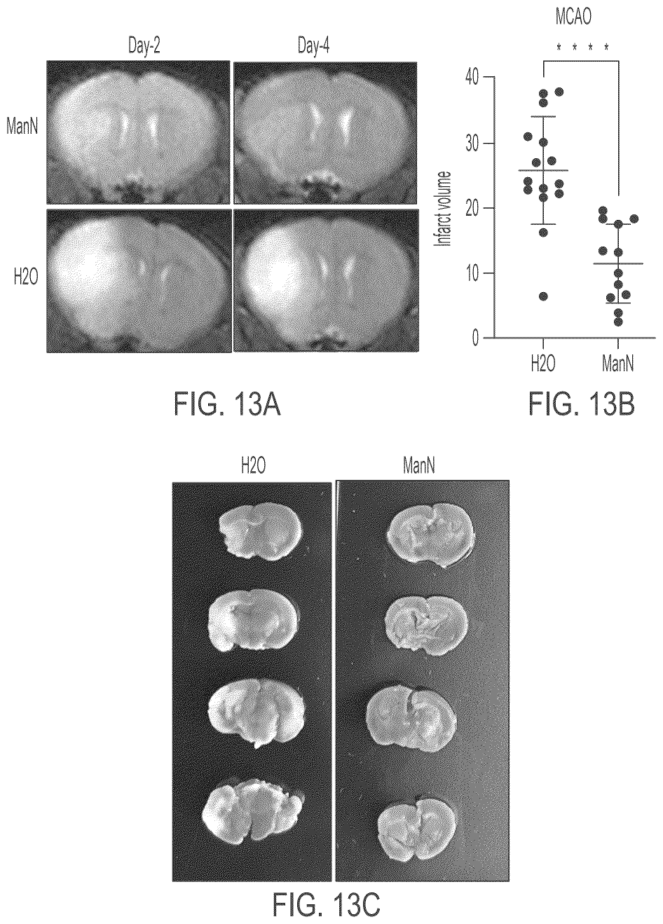

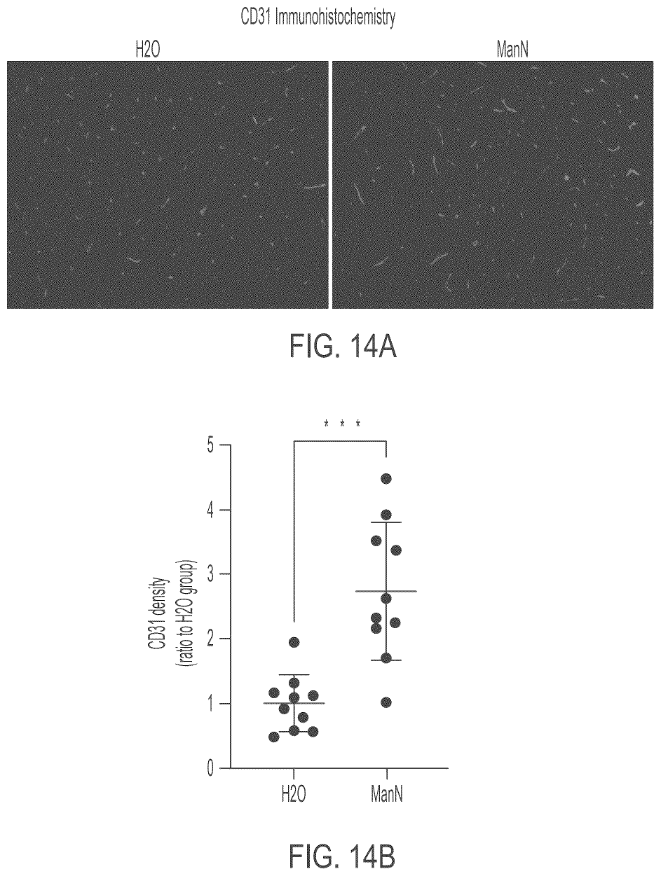

Mice were treated at Day −1 with 40 mg ManN (200 μL of 20% ManN in water) or 200 μL water in control animals delivered by oral galvage. The timing of the pre-treatment of administering the dosage of ManN to the test group mice was about 16 hours prior to surgery. The administration of this dosing regimen was continued once every 48 hours (e.g. at Day 1, at Day 3, etc.) until animals were assessed for the extent of infarction areas in the brain. Approximately 16 hours after the first treatment (at Day 0), animals were subjected to 2 hours of surgical occlusion of the right middle cerebral artery thereby subjecting portions of the right brain to an acute ischemic event. Normative blood flow was restored after the 2-hour period of occlusion. FIG. 12A shows the timing of the ManN administration treatment regimen and the timing of procedures and subsequent assays. FIG. 12B shows the results of a first experiment in which 4 animals were treated with ManN paired with 4 control-treated animals for analysis. MRI results from the coronal plane on Day 4 indicate defined and measurable areas of infarction in ManN-treated and vehicle-treated animals. Multiple coronal slices of CNS from single animals were measured using MRI and calculated for Day 4 total infarct volume fold per animal. ManN-treated animals had a markedly smaller infarction area as represented in the graph. FIG. 12C shows the results of a second experiment in which 4 animals were treated with ManN paired with 4 control-treated animals for analysis. MRI results from the coronal plane on Day 4 indicate defined and measurable areas of infarction in ManN-treated and control-treated animals. Multiple coronal slices of CNS from single animals were measured using MRI and calculated for Day 4 total infarct volume fold per animal. ManN-treated animals had a markedly smaller infarction area as represented in the graph. FIG. 12D shows the combined analysis of data from FIG. 12B-12C demonstrating a statistically significant effect of ManN treatment in reducing overall infarction area as assessed 4 days post-acute ischemic event. Statistical analysis was performed using a two-tailed, two-sample unequal variance t-test. These results demonstrate a measurable benefit in reducing the total infarction area of the brain after an acute ischemic event by a treatment regimen that includes pretreatment and post-treatment with ManN delivered by oral administration. In a testing of additional animals, FIG. 13A shows representative MRI images of the results in animals that were treated with ManN paired with control-treated animals for analysis. MRI results from the coronal plane on Day 2 and Day 4 indicate defined and measurable areas of infarction in ManN-treated and vehicle-treated animals. Reduction in infarct area is noticeable in both Day 2 and Day 4 ManN-treated animals compared with vehicle (H2O)-treated controls. Total Day 4 results are graphed and analyzed in FIG. 13B. Multiple coronal slices of CNS from single animals were measured using MRI and calculated for Day 4 total infarct volume fold per animal. Quantification of MRI images was accomplished using Image J. Each dot represents total calculated CNS infarct volume per animal as measured using MRI. Statistical analysis was performed using a two-tailed, two-sample unequal variance t-test (p<0.001) indicating that a multiple dose treatment regimen comprising administering an effective amount of ManN to mice in the MCAO model significantly reduced CNS infarct volume at Day 4 in ManN-treated animals compared to vehicle-treated animals. In FIG. 13C, TTC (2,3,5-triphenyltetrazolium chloride)-staining was visualized in ManN-treated and vehicle-treated coronal slices of mouse CNS at Day 4 from representative animals. TTC staining is commonly applied for rapid and reliable visualization of hypoxic brain tissue for defining size and area of cerebral infarction. Living cells exposed to TTC staining turn healthy/normal tissue deep red in color. In contrast, damaged or dead tissue remains white demonstrating an absence or reduction in living cells, indicating an area of infarction. The therapeutic benefit constituting a reduction in infarct area in ManN-treated animals at Day 4 is evident with the lack of large regions of white staining in ManN-treated animals compared to extensive CNS regions lacking TTN staining in vehicle-treated animals.

Following the treatment protocol listed in FIG. 12A, animals were tested on Day 6 to examine vascular density within infarct-affected areas of the cerebral cortex by examining CD31 expression. CD31 (PECAM-1) is a marker highly expressed on endothelial cells. CD31 fluorescent immunohistochemistry was used to identify and quantify vascular density within a defined region of cerebral cortex affected by infarct in the MCAO model in ManN-treated and vehicle-treated animals. ManN-treated animals received a therapeutically-effective dosage of ManN (40 mg) administered by oral galvage on Day −1, Day 1, Day3, and Day 5. Vehicle-treated animals received water on Day −1, Day 1, Day3, and Day 5. On Day 6, brain tissue was harvested and fixed. Infarct area was identified and CD31 immunostaining was performed on coronal sections to label the vasculature. Representative CD31 stained sections from a vehicle (H₂O)-treated animal and a ManN-treated animal are shown in FIG. 14A. In FIG. 14B, quantification of vascular density was performed using Image J on representative sections and cumulative results are graphed according to CD31 staining density. Statistical analysis was performed using a two-tailed, two-sample unequal variance t-test (p<0.001) indicating that a multiple dose treatment regimen comprising administering an effective amount of ManN to mice in the MCAO model significantly increased vascular density in a region of cerebral cortex affected by infarct in ManN-treated animals compared to vehicle-treated controls.

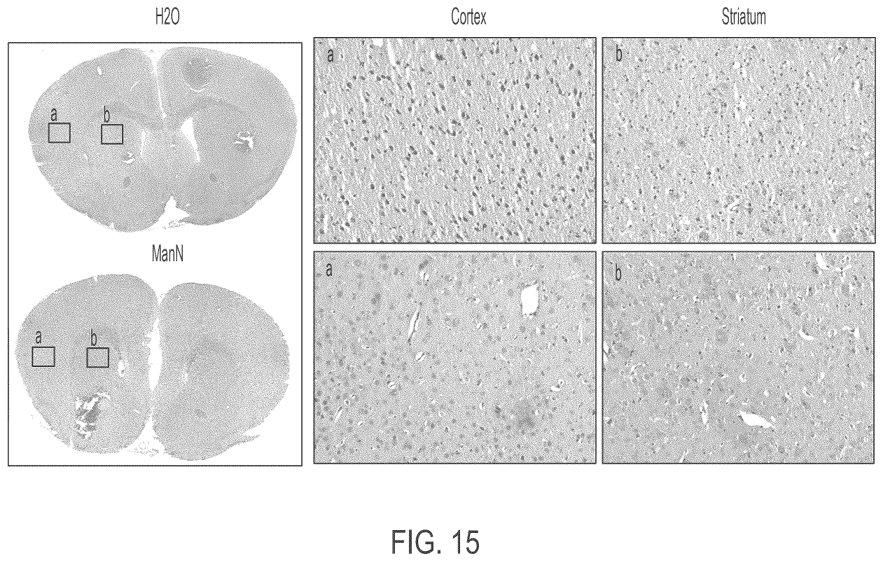

Following the treatment protocol listed in FIG. 12A, animals were tested on Day 4 to examine histopathological alterations within infarct-affected areas of the right hemisphere by examining hematoxylin and cosin (H&E) staining in coronal sections. ManN-treated animals received a therapeutically-effective dosage of ManN (40 mg) administered by oral galvage on Day −1, Day 1, and Day3. Vehicle-treated animals received water on Day −1, Day 1, and Day 3. On Day 4, brain tissue was harvested and fixed, then sectioned and stained using H&E. As seen in on the top row of FIG. 15, regions of cerebral cortex and striatum show alterations of brain morphology and cellular structure in a vehicle-treated mouse brain coronal section. The bottom row of FIG. 15 shows a coronal section of CNS from a ManN-treated mouse with inset regions of cerebral cortex and striatum shown at higher magnification. Vehicle-treated animals showed H&E staining lighter in the infarction area compared with that of normal brain tissue due to the presence of increased vacuolation and edema. Nuclei in the vehicle-treated infarction area were smaller, appeared shrunken, and stained more darkly than nuclei in equivalent ManN-treated brains regions affected by infarction. These results demonstrate that ManN pretreatment and continued post-treatment following an acute ischemic event ameliorates histopathological alterations in the brain of a mouse MCAO model of stroke.

### Materials and Methods

### Small Molecule Library

MSMLS (Mass Spectrometry Metabolite Library of Standards) (IROA TECHNOLOGIES, Bolton, MA; now Sigma) is a collection of 619 high-quality small molecules (purity >95%) that span a broad spectrum of primary metabolites, including carboxylic acids, amino acids, biogenic amines, polyamines, nucleotides, coenzymes, vitamins, lipids, etc. Plates were spun at 300 g after reconstitution, according to the instructions of the manufacturer.

### Chemical Compounds

D-Mannosamine hydrochloride was obtained from Sigma (M4670) or Spectrum Chemical MFG Corp (M3220). 1-Amino-1-deoxy-D-Fructose hydrochloride (D-isoglucosamine) (803278), D-(+)-Galactosamine (1287722), D-(+)-Glucosamine (1294207), N-acetyl-Mannosamine (A8176), N-acetyl-galactosamine (A2795), N-Acetyl-Glucosamine (A8625), Meglumine (M9179), Muramic acid (M2503), N-Acetylneuraminic acid (A2388), D-(+)-Glucose (D9434), D-(+)-Mannose (1375182), Meglumine (M9179), Tunicamycin from Streptomyces sp. (T7765) and SP600125 (S5567) were obtained from Sigma. Hypure cell culture grade water used to dissolve compounds (endotoxin <0.005 EU/ml) was obtained from Hyclone. Axitinib was obtained from Santa Cruz (SC-217679). Tauroursodeoxycholic acid (TUDCA) was from Calbiochem (1180-95-6) and 4-phenylbutyric acid (4-PBA) (P21005), Castanospermine (Cas, C3784), Kifunensine (K1140), DMSO (D2650) were from Sigma. DMSO D2650) was used as a solvent for Cas.

### Antibodies

Antibodies used in the present study were from Cell signaling Technology Inc. (Danvers, MA) unless otherwise specified. Total: VEGFR2 (2479), ERK (4695), p38 (9212), JNK (9252), mTOR (2983), AKT (4691), CREB (9104), CHOP (2895), ACC (3676), ATF-6 (65880), Bip (3183), AMPK (5832), FGFR1 (9740), eNOS (9586), VE-Cadherin (2500), c-Met (3127 or 3148), Neuropilin (3725), CD31 (3528), c-Jun (9165). Phosphor-antibodies: VEGFR2 (Tyr1175, 2478 or 3770), ERK1/2 (Thr202/Tyr204, 4376), p38 (Thr180/Tyr182, 4511), JNK (Thr183/Tyr185, 9251), mTOR (Ser2448, 5536), AKT (Ser473, 4060), CREB (Ser133, 9191), ACC (Ser79, 3661), eNOS (Ser1177, 9571), AMPKa (Thr172, 50081), c-Jun (Ser73, 9164), (31 integrin (4706 & 34971), av integrin (4711), JNK1 (3708), JNK2 (4672), JNK3 (2305). Anti-(3-actin was from Sigma.

### Cells

Primary human umbilical vein endothelial cells (HUVEC, passage 4-10) were obtained from Lonza (C2519AS, Lot #234871) and cultured on 0.1% gelatin-coated plates in endothelial cell growth media (EGM) containing 2% FBS, BBE (Bovine Brain Extract), heparin, human EGF, hydrocortisone, ascorbic acid, GA-1000 (Gentamycin, Amphotericin B) and VEGF. Bovine retinal microvascular endothelial cells (BRECs, #BRMVEC-3) and bovine choroidal microvascular endothelial cells (BCECs, #BCME-4), both from VEC Technologies (Renssellaer, NY), were maintained in fibronectin-coated plates (1 μg/cm2). The growth medium was low glucose DMEM, supplemented with 10% bovine calf serum (BCS), 5 ng/ml bFGF and 10 ng/ml human VEGF165. Cells were maintained at 37° C. in a humidified atmosphere with 5% CO2. bFGF (233-FB) and VEGF165 (293-VE) were purchased from R&D systems. Human retinal microvascular endothelial cells (passage <15) was from Cell Systems Corporation (Kirkland, WA). They were grown on 0.1% Gelatin-coated plates in Medium 131 containing 5% fetal bovine serum, hydrocortisone (1 μg/ml), human fibroblast growth factor (3 ng/ml), heparin (10 μg/ml), human epidermal growth factor (1 ng/ml) and dibutyryl cyclic AMP (0.08 mM) (MVGS, S 005-25, Gibco Invitrogen). The human RPE cell line ARPE-19 was from the ATCC. Cells were gently lifted in 0.025% trypsin and plated in RtEGM media (Clonetics) containing 2% FBS, L-glutamine, human bFGF, GA-1000). Once cells attached to plates, serum free RtEGM media was used to maintain the culture for best result. ARPE-19 was obtained from ATCC (CRL-2302) and cultured according to company's instruction. NIH3T3 cells were obtained from ATCC (CRL-1658). Human adult dermal MVECs (CC-2543) were cultured in EGM-2 MV (CC-4147, Lonza). Keratinocytes (ATCC, PCS-200-011) were cultured in dermal cell basal media (PCS-200-030) plus keratinocyte growth kit (PCS-200-040). Human primary dermal fibroblasts (ATCC PCS-201-012) were cultured in fibroblast basal medium (ATCC, PCS-201-030) plus growth kit (ATCC, PCS-201-040). Growth stimulators used in the assay included human EGF (R&D systems, 236-EG), murine TGF13 (R&D systems, 410-MT), KGF (Sigma, K1757), or 10% FBS growth media. 4T1 cells were obtained from the ATCC (CRL-2539) and cultured in RPMI-1640 with 10% FBS (Omega Scientific, Tarzana, CA) and antibiotics. A673 (CRL-1598), A549 (CCL-185), U87MG (HTB-14) cells were from ATCC and cultured in high glucose DMEM containing 10% FBS. FBS (S12550) was purchased from R&D systems. BCS (SH30073.03) was obtained from Hyclone. All cell lines used in the study are negative for mycoplasma contamination by various vendors.

### Cell Proliferation Assays

Proliferation assays with BCECs and BRECs were performed. Log-phase growing BCECs or BRECs (passage <10) were trypsinized, re-suspended and seeded in 96-well plates (no coating) in low-glucose DMEM supplemented with 10% bovine calf serum, 2 mM glutamine, and antibiotics (growth medium), at a density of 1200-1500 cells per well in 200 μl volume. All reagents were added at the indicated final concentrations. After 3-6 days, cells were incubated with AlamarBlue for 4 hrs. Fluorescence was measured at 530 nm excitation wavelength and 590 nm emission wavelength. The experiments were repeated at least three times. To create a hypoxic condition, cells were placed in a hypoxia incubator with a mixture of gas consisting of 1% O2, 5% CO2 and 94% N2. On each 96-well plate, untreated and VEGF-treated (10/ng/ml) wells were included to monitor plate-to-plate variations. 20% methanol or 0.05% DMSO served as negative controls. 0.05% DMSO served as negative controls when Cas was tested in these cells. Human RMVECs and human adult DMVECs were split into Gelatin-coated 96 wells (2000 cells per well) in low glucose DMEM containing 10% FBS. 1200 cells/well was set up for proliferation assay in low glucose media containing 0.5% FBS. Data was collected at day 4 or 5. HUVEC (p7-10) were grown on gelatin-coated plate until it reached 70-80% confluency.

On the day of the assay, cells were dissociated with 0.05% trypsin, which was neutralized with 0.5% FBS-containing EBM. Cells were briefly spun and then re-suspended in 0.5% FBS-media. Cells were counted and plated in 96-well, 1000 cells/well. Triplicate wells were used for each treatment. Data were collected at day 3, and then cells were fixed in 4% paraformaldehyde for 15 min before adding crystal violet. Cell-covered areas were quantified after taking pictures by Image J software.

Proliferation assays with fibroblasts were done in low-glucose DMEM containing 1% FBS, with or without 10 ng/ml bFGF, or 100 ng/ml human EGF and the assay was ended at day 3. ARPE-19 cells were gently lifted with 0.025% trypsin and plated in RtEGM media (Clonetics) containing 2% FBS, L-glutamine, human bFGF and GA-1000. Once cells were attached to plates, serum-free RtEGM media was used to maintain the cultures. For proliferation assays, 1500 human RPE cells were plated into 96-well plates in low-glucose DMEM containing 1% FBS. A673, U87MG, Calu6 and AML12 cells were grown until confluent and were then harvested and re-suspended in appropriate assay media. For proliferation assays, cells were plated at the density of 1000-2000 cells/well in low-glucose DMEM containing 5% FBS or otherwise stated. Bovine pituitary cells (pituitary folliculostellate cells) were isolated as previously described. For proliferation assays with human epidermal keratinocytes, human DMVECs and human dermal fibroblast cells, 1000 cells/well were plated in low-glucose DMEM containing 1% FBS with or without various growth factors. The assay was ended at day 3 for bovine pituitary cells and at day 4 for all the other cell types. For 4T1, 1000 cells were plated in RPMI-1640 with 2% basement membrane extract (BME) and 2% FBS on BME-coated 96-wells and treated 4 hrs later. Four days later, tumor cell growth was measured by the MTS assay (Promega, Madison, WI), a colorimetric assay that measures metabolic activity of viable cells. Recombinant human transferrin was obtained from EMD Millipore (Temecula, CA). Recombinant mouse apo-transferrin was obtained from Sigma.

### Sirna Knockdown

BCECs were plated in 6-well culture plates at a density of 1.5×105 cells/well and cultured overnight. 2 ml of antibiotics-free culture medium was used to replace the old medium. siRNAs, including siNegative (Ambion, AM4611), siRNA against JNK1 #2 (Invitrogen, NM_001192974.2_siRNA 266), JNK1 #4 (Invitrogen, NM_001192974.2_siRNA_485), siRNA against JNK2 #2 (Invitrogen, XM_005208371.4_siRNA_1240), JNK2 #4 (Invitrogen, XM_005208371.4_siRNA_696), were mixed with Lipofectamine RNAiMAX reagent (ThermoFisher Scientific, 13778150) in Opti-MEM I Reduced Serum Medium (Gibco, 31985062) according to manufacturer's instructions. Briefly, a mix containing 25 pmol of siRNA, 7.5 μL of RNAiMAX reagent and 125 μL of Opti-MEM medium was used to transfect cells in each well, to a final siRNA concentration of 12.5 nM. A mix of RNAiMAX and Opti-MEM was used as no siRNA control. Cells were incubated with siRNAs. 8 hrs later, the siRNA-containing medium was replaced with fresh medium. 24 and/or 48 hrs after transfection with siRNAs, cells were used for proliferation assays and protein extraction.

### Pngase F Treatment

Glycerol-free PNGase F was obtained from New England Biolabs (Ipswich, MA). Briefly, BCECs were lysed with NP-40 containing proteinase inhibitors (Thermo Scientific, Waltham, MA). Lysates were cleared at 4° C. at 5000 g for 25 mins. Total protein content was measured using Pierce BCA protein assay kit (Thermo Scientific). 20 mg of protein was mixed with 10× denaturing buffer and H2O to a total volume of 10 ml. Glycoproteins were denatured at 100° C. for 10 mins, followed by adding Glycobuffer and PNGase F. The reaction was carried out at 37° C. for 2 hr.

### Western Blots

Cells were allowed to reach-80% confluency in 12-well plates. Cells were pre-treated with ManN, Kif or Cas for various time durations, with or without the subsequent addition of VEGF, with H2O as the solvent control for ManN. At various time points, plates were taken out of the incubator and kept on ice. Cell monolayers were first washed once with ice-cold PBS before lysis with 250 μI of Pierce RIPA buffer (ThermoFisher Scientific, Rockford, IL) or use 50 mM Tris-HCl (pH 7.6), 150 mM NaCl, 10% Glycerol, 1% NP-40 containing protease/Phosphatase inhibitor cocktail (100×) (Cell signaling, #5872). Lysates were collected and mixed with 4× Bolt LDS Sample Buffer (Novex, Carlsbad, CA) in the presence of Halt protease inhibitors and phosphatase inhibitor cocktail (ThermoFisher scientific, #NP0007). The samples were subjected to SDS-PAGE (Bolt 4-12% Bis-Tris Plus, Invitrogen) using Bolt MES SDS running buffer or NuPAGE 3-8% Tris-Acetate gel using Tris-Acetate SDS running buffer (Novex). HUVECs (passage 6-8) were plated in EBM-2 basal medium (Lonza) with 0.2% FBS. Following overnight culture, cells were serum-starved in EBM-2 medium for 4 hrs prior to treatment with 50 ng/ml of VEGF165 or vehicle controls for various lengths. Equal amounts of protein lysates were analyzed by SDS-PAGE and blotted with the indicated antibodies. Proteins were transferred using Tris-Glycine buffer with 20% Methanol (Proteonomics grade) (Apex BioResearch Products). Membranes were first incubated with 5% milk in TBST, pH 7.6 (TEKnova, Hollister, CA), followed by blotting with primary and secondary antibodies. ECL anti-rabbit IgG, horseradish peroxidase linked whole antibody from donkey or sheep anti-mouse were obtained from GE Healthcare (UK limited). SuperSignal West Dura Extended Duration substrate was from ThermoFisher Scientific. In some cases, the same PVDF membranes were stripped by 8-min incubation in the Restore Plus Western Blot Stripping Buffer (ThermoFisher Scientific) to show total specific protein expression, followed by second stripping for (3-actin expression.

### Migration Assays

HUVECs (passage 6-8) were cultured and serum-starved as described in “Western Blots”. Ten thousand cells in 150 mL of EBM-2 medium were then added to the upper chamber of 8 gm pore size cell culture inserts (Falcon) coated with 0.1% gelatin. The lower compartment was filled with 600 mL EBM-2 medium containing various agents. The plates were incubated at 37° C. to allow migration. After 4 hrs, cells were fixed with 4% PFA for 20 min and then stained with crystal violet (Sigma-Aldrich) for 20 min at RT. Migrated cells on the bottom side of the insert membrane were quantified by counting whole area of the insert at 40× magnification. The experiments were carried out in triplicate and repeated three times. BCEC migration was set up similarly, except that wells were coated with FN, cells were suspended in 1% serum media and migration time was 18-24 hrs.

### Scratch Assay

BCECs (passage 6-10) and HUVEC (passage 6-8) were used in this assay.

Cells were grown until about 80% confluency in 6-well plates, washed twice with PBS and then starved in serum-free DMEM (low Glucose, Hyclone) for 5 hrs before making a “scratch” using 1 ml tip. Cell monolayers were briefly washed once with serum-free media, followed by various treatments in media containing 1% FBS. 48 hrs later, the assay was stopped by adding 2 ml 4% paraformaldehyde. 20 min later, fixed cells were stained with 1 ml Crystal Violet (Sigma). Plates were washed gently under the running tap water and air-dried before taking pictures. Images were acquired by ZEISS Discovery V8 SteREO microscopy equipped with PixeLINK Megapixel FireWire camera. Quantification of wound closure was done using AxioVision LE Rel.4.4 software. Six images were taken for each sample and six measurements (in pixel) were made on each image using AxioVision LE Re1.4.4 software.

### N-Glycan, Monosaccharide, Sialic Acid, O-Glycan Analysis

As soon as BCECs reached about 80% confluency, they were washed twice with phosphate buffered saline (PBS, Sigma) and harvested by scraping. The cells were pelleted by centrifugation at 300 g for 3 mins and washed once with cold PBS. Cells were homogenized and total protein was measured. All subsequent analysis was based on known protein amount.

N-linked glycans were removed from glycoprotein samples using PNGase-F kit (New England BioLabs, P0705S). Briefly, 300 μg of protein sample was reconstituted in 180 μl UltraPure water. 20 μl of 10× denaturing buffer was added and boiled using 100° C. water bath for 14 mins. Samples were cooled down to room temperature and centrifuged at 2700 g for 1 min. Subsequently, 50 μl 10×NP-40 was added and samples were kept at room temperature for 30 mins with vortexing at 5 min interval, followed by adding 25 μL of 10× reaction buffer and mixing thoroughly. 5 μl PNGaseF (2500 U) was then added to the samples and mixed gently. Samples were incubated at 37° C. for 16 hrs. Released N-glycans were purified using solid phase extraction method. Briefly, N-glycans were purified by passing the reaction mixture sequentially over pre-conditioned Sep-Pak C18 1 cc cartridge (Waters) and HyperSep PGC (poly graphitized charcoal) cartridge (25 mg, 1 ml Thermo Scientific). The cartridge was washed with 4 ml of water and the PGC alone was washed with additional 1 ml of water. N-glycans bound to PGC were eluted using 30% acetonitrile containing 0.1% TFA in water. Finally, purified N-glycans were lyophilized and labeled with 2-AB. Briefly, samples were dissolved in 10 μl solution of 0.44 M 2-AB (2-Amino benzamide) in 35% acetic acid in DMSO containing IM sodium cyanoborohydride. The samples were incubated at 65° C. for 2.5 hrs. The 2-AB labeled glycans were purified using GlycoClean S cartridge (GLYKO) following their glycan clean-up protocol. Excess reagent was removed from the samples using Glycoclean S-cartridge (Prozyme) and labelled glycans were dried using SpeedVac and stored at −20° C. Profiling of 2-AB labeled glycans was obtained using Dionex CarboPac PAI (4×250 mm) anion exchange column along with a guard column (4×50 mm) at flow rate of 1 ml/min. Glycans were separated in 100 mM sodium hydroxide with a sodium acetate gradient of 0-250 mM in 0-75 minutes. The data was collected using the Dionex ICS-3000 HPLC system with Ultimate 3000 fluorescence detector (Dionex) set at Acx 330 nm at Atm 420 nm with sensitivity 7. The data was processed using Chromeleon software (Thermo Scientific).

Monosaccharide composition analysis was done using HPAEC-PAD (Thermo-Dionex ICS3000) and nmole amount of each monosaccharide present in 25 μg of protein was calculated. Samples were hydrolyzed using 2 N trifluoroacetic acid (TFA) at 100° C. for 4 hrs. Followed by removal of acid using dry nitrogen flush. To ensure complete removal of acid, samples were co-evaporated twice with 100 μl of 50% isopropyl alcohol (IPA). Finally, the samples were dissolved in Milli-Q water and injected on HPAEC-PAD. Monosaccharide profile was done using Dionex CarboPac PAI column (250 mm×4 mm; with 50 mm×4 mm guard column). An isocratic solvent mixture of 19 mM sodium hydroxide with 0.95 mM sodium acetate was used at a flow rate of 1 ml per minute for 25 mins. Data were acquired using manufacture supplied standard Quad waveform for carbohydrates. All neutral and amino sugars were identified and quantified by comparing with authentic monosaccharide standard mixture consisting of L-fucose, D-galactosamine, D-glucosamine, D-galactose, D-glucose and D-mannosc.

Mild acid hydrolysis was used to release sialic acid. Briefly, samples were treated with 2 M acetic acid at 80° C. for 3 hrs followed by removal of excess acid using speed vacuum. Sialic acid was then tagged with DMB reagent and analysis was done using RP-UPLC-FL (Waters Acquity UPLC) system. Known amount of standard Neu5Ac was used to quantify amount of sialic acid in samples.

For O-glycan analysis, homogenized cell samples were treated with 50 mM NaOH in presence of 1M NaBH4 for 16 hrs at 45° C. The reaction mixture was neutralized using ice-cold 30% acetic acid slowly. The neutralized reaction mixture was then passed over Dowex 50-X cation exchange resin to remove sodium ion and lyophilized. Excess boric acid generated during neutralization was then removed by co-evaporation using acidified methanol and methanol respectively. Finally the O-glycan was purified by passing over C18 cartridge. Dried and purified O-glycan was then methylated and used for O-glycan analysis after permethylation. Permethylated samples were then dissolved in absolute methanol and mixed with SDHB (Super-DHB) MALDI matrix in 1:1 v/v ratio and spotted on maldi plate. Mass spectral data was acquired using Bruker AutoFlex mass spectrometer at positive, reflectron mode. The mass spectral data were analyzed and annotated using GlycoWork Bench software and masses matched with the proposed structures were annotated. The mono-isotopic ion intensities are taken for calculation.

To measure cellular uptake of ManN and subsequent conversion to ManN-6P, BCECs were grown in 60 mm dishes to a density of −6×105 cells per dish. ManN was added to cultures at the final concentration of 400 μM. Cells were then incubated for 2 hrs. Monolayers were washed three times with PBS at room temperature and lifted by a cell scraper on ice in 10 ml PBS. Cell pellets were obtained by centrifugation at 400 g for 5 mins and stored at −80° C. for further use. Cell pellets were suspended in 200 μl of Ultra-pure ice-cold water in presence of 1 μl of protease inhibitor. Cells were sonicated for 1 min with 30 sec pulses and vortexed to form homogeneous solution. 2.5 μl of the homogenate was used for protein estimation using BCA-assay method in triplicate. A standard curve of BSA at concentration between 0-800 μg/ml was done to quantitate the total protein amount. The cell homogenate was filtered through pre-washed 3K filters and the filtrate was dried using speed-vac. The dry sample was reconstituted in 100 μl of ultrapure water and sample with 200 μg equivalent of protein was injected onto HPAEC-PAD. A known amount (1 nmol) of ManN, Glucose, Mannose, and ManN-6P standards were used to quantify the sugars present in the samples. All standards, except ManNH2-6P, were obtained from Sigma-Aldrich. ManNH2-6P was from Omicron Biochemicals, Inc (South Bend, IN). The amounts of monosaccharides present in different cells are presented as nmol/mg of total protein amount. All analyses was performed in a Thermo-Dionex ICS system using a CarboPac-PA-1 column in 100 mM NaOH and 250 mM NaOAc as HPLC running buffer.

### Biotinylation of Surface Proteins

BCECs were plated in 10 cm cell culture dishes 3 days prior to the cell surface protein isolation. Cells were washed three times with Dulbecco's PBS with CaCl2 and MgCl2, followed by a 30 min incubation with EZ-Link Sulfo-NHS-SS-Biotin (Pierce, Rockford, IL, USA; 0.5 mg/ml in Dulbecco) on ice. Cells were washed twice with Dulbecco and the non-reacted biotin was blocked with 20 mM glycine for 15 mins. To prevent the reduction of the disulfide bridge in the biotin molecule during the cell lysis process, a 100 μM oxidized glutathione (Sigma-Aldrich, St. Louis, MO) was added in the last wash solution. For cell lysis 500 μL of lysis buffer (2% NP-40, 1% Triton X-100, 10% glycerol, 100 μM oxidized glutathione, EDTA free protease inhibitor tablet (Roche, Mannheim, Germany) in PBS was added to the cells. Lysed cell extracts were scraped off the plates and transferred to an Eppendorf tube followed by incubation on ice on a shaker for 30 mins. The cell extracts were incubated with 30 U of DNase (22° C. 50 mins, Roche, Mannheim, Germany) and centrifuged for 20 mins (20,800×g, at 4° C.) to pellet the insoluble material. The protein concentration of the supernatants was determined. Equal amounts of protein (−2 mg) from each extract were used for cell surface protein isolation. The supernatant was pre-cleared using biotin agarose beads (Pierce ImmunoPure Immobilized D-biotin, Thermo Scientific, 20221) and pre-cleared solution was used for the cell surface protein isolation using streptavidin beads. Beads were washed four times with the lysis buffer, four times with 300 mM NaCl in lysis buffer and twice with 50 mM Tris-HCl, pH 7.8. Proteins were eluted twice with an elution buffer (50 mM DTT in 50 mM Tris-HCl, pH 7.8) at 30° C., followed by pooling of the elutes. Three biological replicates and one non-biotinylated control were used in the study.

### Gene Expression Analysis by Real-Time Q PCR

RNA was prepared using the RNeasy Mini Kit (Qiagen). Fifty ng of total RNA per reaction was used for the real-time PCR (Taqman) analysis. Reactions were set up in MicroAmp Fast Optical 96-well reaction plate, seal with MicroAmp optical Adhesion film and run on ViiA7 Real time PCR system (Applied Biosystems) and the absolute quantification with standard curve was used with Sequence Detection System (SDS) software. The expression level of each gene was further quantified relative to the housekeeping gene RPL19 in the same sample. Taqman primers and probe mixes were obtained from Thermo Fisher Scientific. Bovine VEGF-A (Bt03213282), bovine RPL19 (Bt03229687) and bovine specific VEGFR2 (Bt03258877), GLUT1 (Bt03215313) and GLUT4 (Bt03215316).

### α-Mannosidase, α- and β-Glucosidase Activity Assays

a-mannosidase activity was measured using substrate p-nitrophenyl α-mannopyranoside (1 mM). Enzyme from Jack Bean (M7257) (final concentration of 0.077 U) was incubated at 37° C. in a final volume of 50 μl of 50 mM potassium phosphate buffer, pH 7.5. α-glucosidase was assayed with substrate p-nitrophenyl a-glucoside (7 mM). Enzyme from Saccharomyces cerevisiae-type 1 (Sigma, G5003) (final concentration of 0.1 U) was incubated at 37° C. in a final volume of 50 μl of PBS, pH 7.5. β-Glucosidase was assayed with substrate 4-nitrophenyl β-D-glucopyranoside (Roche). Enzyme from almond (Sigma, G0395) (final concentration of 0.002 U) was incubated at 37° C. in a final volume of 50 μl of PBS, pH 7.5 containing 1% SDS. The incubation was stopped by addition of an equal volume of acid-based stop solution (R&D systems, 895032). Enzymatic activity was measured at 405 nm. α-glucosidase from Saccharomyces cerevisiae type I (G5003) with p-nitrophenyl α-D-glucopyranoside (Sigma, N1377) as the substrate.

Measurement of ManN in Wound Fluid from S. aureus Infected Mice

To test ManN stability in wound fluid collected from mice with skin infection as described below, 1.5 μl of 5% ManN solution was added to each 200 μl wound fluid which was first diluted 1:1 (v/v) with PBS. At each time point, samples were taken from 37° C. incubator and stored at −80° C. Plasma proteins were precipitated by adding ice cold acetonitrile to plasma: acetonitrile 1:3 (v/v) ratio. Samples were kept over ice for 1 hr and then centrifuged at 12000 g for 10 min at 7° C. to form a pellet. Supernatants were transferred to other tubed, dried down on a Speed Vac and then reconstituted in UltraPure distilled water and filtered through pre-washed Nanosep 3K Omega filters (Pall Corporation). The filtrate was dried down on Speed Vac. The dry samples were dissolved in 100 μl of water and 2 μl plasma or wound fluid sample was subjected to HPLC analysis. Neutral and amino sugars were separated on a Dionex CarboPac PAI column 4 mm×250 mm with 4 mm×50 mm guard column. An isocratic gradient of 19 mM sodium hydroxide with 0.95 mM sodium acetate was used at a flow rate of 1 ml/min with 20 min run. Data was collected using the Dionex ICS-3000 HPLC system with pulsed amperometric detector using standard Quad waveform. ManN was identified and quantified by comparison with monosaccharide standard using Thermo Scientific Chromeleon software. No ManN samples served as negative controls.

### Skin Wound Healing Model

All animal experimental procedures were approved by the Institutional Animal Care and Use Committee (IACUC) of the University of California San Diego and conducted in an ethical fashion and in accordance with the guidelines of the Animal Care Program (ACP).

The model has been previously described. Briefly, C57BL/6 female mice (8-10 weeks old) were obtained from Jackson labs (Sacramento, CA). A fresh, full-thickness punch wound (4 mm diameter) using a punch (Acu Punch, Acuderm inc. Ft. Lauderdale, FL) splinted with a sterile neoprene ring (6-mm outer diameter and 4-mm inner diameter), fastened with 5-6 sutures (4-0 nylon) under the influence of Isoflurane was created on the back of the animal in a Class II Biological Safety Cabinet. For all surgical procedures, sterile technique was followed. Buprenorphine was given subcutaneously prior to awakening from anesthesia for anticipated pain. Mice were monitored until fully awake and were housed individually to minimize damage/biting/fighting to the surgical site. Recombinant human VEGF was a gift from Roche-Genentech (Telbermin, recombinant human VEGF165). Treatment agents were prepared in PBS, sterile-filtered and 25 μl solution was applied daily directly to a wound bed for the first 4-5 days under the influence of Isoflurane, followed by daily observation. Wound closure was monitored by regular imaging and the wound area was quantified using ImageJ (National Institutes of Health, Bethesda, MD, USA).

At day 4 after wounding, wounds were excised with a 2 mm rim of surrounding tissue and placed in 10% formalin for a maximum of 24 hrs. The wounds were then bisected down the center, and 5-μm paraffin sections were processed for Hematoxylin and Eosin (H&E) and Masson's Trichrome staining. Epithelial gap was measured histomorphometrically using AxioVision LE Rel.4.4 software. The skin tissues were fixed in 10% formalin for 24 hrs. Paraffin embedded and sectioning were performed by the UCSD, Moores Cancer Center Histology Core. The 5-μm paraffin sections were deparaffmized and rehydrated before heat-induced antigen retrieval was performed in 10 mM citrate buffer (pH 6.0). Immunostaining was performed as previously described. Anti-CD31 (SZ31, rat IgG2a) (Dianova, Warburgstrasse 45, 20354 Hamburg, Germany) was used at 2 μg/ml. Small vessels stained positive for CD31 were counted microscopically on 10 fields (20×) taken around the wound.

### Vascular Permeability Assay

Vascular permeability was assessed using a modified Miles assay. Hairless male guinea pigs (Crl: HA-Hrhr/IAF, 75 days old, 450-500 g, Charles River Laboratories) were anesthetized by intraperitoneal (i.p.) administration of xylazine (5 mg/kg) and ketamine (75 mg/kg). The animals then received an intravenous injection (penile vein) of 1 ml of 1% Evans blue dye. After 15 mins, intradermal injections (0.05 ml/per site) of different doses of ManN were administrated into the area of trunk posterior to the shoulder. All reagents were diluted in PBS for intradermal administration. 25 ng of VEGF165 per site was used as positive control. 30 mins after the intradermal injections, animals were euthanized by i.p. injection of pentobarbital (200 mg/kg). Skin tissues were dissected from the connective tissues and photographed.

### Murine Skin Infection Model

A skin infection model in the mouse was established. Briefly, mid-log phase of Staphylococcus aureus sub-cultured from overnight cultures in Todd Hewitt broth were used in this study. 6-8 weeks old C57BL/6 mice were obtained from Charles River Laboratories. Mice were shaved and depilated by Nair cream before infection. 5×107 CFU of S. aureus was intradermally injected into the left groins of mice. After 3 days, abscesses were surgically removed and homogenized on ice. Fluid was collected and spun at 14000 rpm. Cleared supernatant was diluted with PBS 1:1 for further use. Animals were housed in clean cages and experimental procedures hereafter were carried out under pathogen-free conditions. The presence of bacteria in the wound fluid was confirmed using Todd Hewitt Broth (THB) plates.

### Hindlimb Ischemia Model and Evaluation of Blood Flow

C57BL/6 male mice (6-8 weeks old) were subjected to unilateral hindlimb surgery under anesthesia with ketamine/xylazine cocktail. Briefly, the left femoral artery was separated from the vein and nerve, ligated proximally, and excised. The right hindlimb served as control. Blood flow was measured by using a laser Doppler perfusion imager (PeriScan PSI; Perimed). Ischemic and nonischemic limb perfusion was measured before and after surgery and 1, 2 and 3 weeks later. After surgery, mice were randomly allocated to different groups (8 mice for each group). 200 μl of 20% ManN was orally administrated every other day from 3rd day after surgery. 200 μl of 1 mg/nil Kif was administrated through ip injection every other day. H2O was used as a vehicle control. The final blood flow values were expressed as the ratio of ischemic to nonischemic hindlimb perfusion from the same animal. Quantification of blood vessel area was carried out as described.

### Retinal Neovascularization

Assessment of retinal angiogenesis following intravitreal administration was done. Briefly, 6-8 weeks old C57BL/6 male mice were randomly allocated to different groups and anesthetized with ketamine/Xylazine cocktail. The indicated amounts of ManN, Kif or bFGF (R&D systems, AF-233-NA) in 1 μl of PBS and PBS vehicle control were injected intravitreally with a 33-gauge Hamilton syringe. Seven days after injection, animals were euthanized. Eyes were then enucleated and fixed in 4% paraformaldehyde (PFA) for 30 mins. Retinas were separated and stained with anti-CD31 immunofluorescence (IF) to evidence the vasculature. Evaluations were performed by an investigator blinded to the treatment. For CD31 IF, rat anti-mouse antibody (BD Biosciences, CAT #550274) was diluted 1:100 and incubated overnight at 4° C. After 4-hour incubation with the Alexa Fluor-488-conjugated anti-rat antibody (Life Technologies, A11006), whole mounts were imaged via the 488 nm channel using AIR Confocal STORM super-resolution system (Nikon). Quantification of vascular density in choroids and retina was carried out by Image J. Each experiment was repeated three times with similar results, and each treatment group consists of 5 individual samples.

### Middle Cerebral Artery Occlusion Stroke Model

Assessment of extent of infarction size in the brain as modulated by ManN treatment was carried out. A mouse model of acute stroke was chosen to investigate the effects of ManN pretreatment and continued post-treatment of the causative ischemic evident. This experiment was performed in duplicate. Adult C57BL/6J mice (Jackson Laboratory, Bar Harbor, ME) weighing between 20-25 g were allocated randomly to each group. One day before surgery (Day −1), hexosamine D-mannosamine (ManN) treatment was begun. The drug (ManN) was administered by oral gavage; 40 mg was given to each mouse (200 μL of 20% ManN in water). Water was given each time as vehicle control in the control group mice. This treatment regimen was repeated every 48 hours (e.g. on Day 1, Day 3, etc.) until animals were examined for the extent of infarction area. Surgery was carried out as previous described. Briefly, at Day 0, mice were anesthetized with ketamine/xylazine cocktail. Mice were then put in the supine position and prepared in a sterile manner. A midline neck incision was made and the soft tissues over the trachea were retracted gently. The left common carotid artery (CCA) was carefully exposed and isolated. Following double ligation of the proximal end of CCA, the external carotid artery (ECA) was ligated to prevent bleeding and the internal carotid artery (ICA) was temporarily occluded with a microartery clamp. A small incision was made 2 mm distal to the ECA-CCA branch and a 6-0 silicone-coated rounded-tip monofilament nylon suture (6022910PK10Rc, Doccol Corporation, MA, USA) was gently inserted through CCA. After the removal of the microartery clamp on ICA, filament was advanced into the ICA until it blocked the origin of the middle cerebral artery (approximately 9-10 mm). After 2 hours occlusion, the suture was removed to restore blood supply of the MCA territory. Body temperature was maintained at 36.5-37.5° C. using a heating pad on the surgical table throughout the procedure from the start of the surgery until the animals revived from anesthesia. For sham operated animals, vessel isolation was performed without suture insertion. One day after surgery, the neural score was rated based on the behavior of mice. The MRI imaging of brain will be carried out 24 hours after reperfusion and every other day until determined end points are reached. T2 weighted (T2w) imaging was performed for brain infarction. 10 slices with a thickness of 1 mm and a field of view of 2×2 cm were positioned over the brain. Alternatively, the brain of a mouse is collected 24 hours after reperfusion to carry out TTC staining. Slices of the brain are taken in the coronal plane at 1 mm intervals. Fresh brain slices are put in 1% TTC solution to determine the infarction area.

### Statistics and Reproductivity

Statistical parameters including the n values, are indicated in the figure legends. The sample size was determined to ensure adequate power, as recommended by the Biostatistics and Bioinformatics Department, Moores Cancer Center. We used 2-tailed, two-sample unequal variance t test. Statistical significance was further confirmed using Wilcoxon rank-sum test between treatment groups of interest for some of the in vitro data sets as the method does not need the normal assumption on variables. Statistical inference was based on the p-value of each comparison using R function “wilcox.test”. We used linear mixed effects (LME) model to investigate the wound area (in percentage), between three treatment groups (ManN, VEGF, VEGF+ManN) and PBS group. Two LME models were fitted. In the first LME model, the controlled group was considered as the reference group. We included day effect (considered days as categorical variable instead of continuous variable), and its interaction with treatment as fixed effects, and we included subject id as the random effect to involve correlations among measurements on different days for the same subject. There's no difference among different groups at baseline. In the second LME model, we relevel group VEGF+ManN as the reference group in order to investigate comparisons between single treatment groups and combination treatment. For each LME model, we explored different treatment effects and their corresponding p-values on Day 3, Day 5 and Day 8 with respect to the reference group respectively. Data are considered significant when p<0.05. Significant p values are represented in the figures as follows: p<0.001, **p<0.01, *p<0.05. For each experiment, a representative experimental result is shown from 2-5 independent studies.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### Aspects

Aspect 1: A pharmaceutical composition for treating stroke, the pharmaceutical composition comprising an effective amount of an N-glycosylation inhibitor administered to a subject in need thereof.

Aspect 2: The pharmaceutical composition of aspect 1, wherein the N-glycosylation inhibitor comprises hexosamine D-mannosamine (ManN), Kifunensine (Kif), Castanospermine (Cas), or a combination thereof.

Aspect 3: The pharmaceutical composition of aspect 1, wherein the N-glycosylation inhibitor comprises ManN.

Aspect 4: The pharmaceutical composition of any one of aspects 1-3, wherein the subject has experienced an ischemic stroke.

Aspect 5: The pharmaceutical composition of aspect 4, wherein the ischemic stroke comprises a thrombotic stroke or an embolic stroke.

Aspect 6: The pharmaceutical composition of any one of aspects 1-3, wherein the subject has experienced a hemorrhagic stroke.

Aspect 7: The pharmaceutical composition of aspect 6, wherein the hemorrhagic stroke comprises a subarachnoid hemorrhage or an intracerebral hemorrhage.

Aspect 8: The pharmaceutical composition of any one of aspects 1-3, wherein the subject has experienced a brainstem stroke.

Aspect 9: The pharmaceutical composition of any one of aspects 1-3, wherein the subject has experienced a transient ischemic attack.

Aspect 10: The pharmaceutical composition of any one of aspects 1-3, wherein the subject has experienced a cryptogenic stroke.

Aspect 11: The pharmaceutical composition of any one of aspects 1-10, wherein the effective amount of an N-glycosylation inhibitor comprises a single dosing treatment regimen.

Aspect 12: The pharmaceutical composition of aspect 11, wherein the single dosing treatment regimen comprises pretreatment, concurrent treatment, or post-treatment in relation to an ischemic event affecting the brain.

Aspect 13: The pharmaceutical composition of any one of aspects 1-10, wherein the effective amount of an N-glycosylation inhibitor comprises a multiple dosing treatment regimen.

Aspect 14: The pharmaceutical composition of aspect 13, wherein the multiple dosing treatment regimen comprises pretreatment, concurrent treatment, and/or post-treatment in relation to an ischemic event affecting the brain.

Aspect 15: The pharmaceutical composition of any one of aspects 1-14, wherein the effective amount of an N-glycosylation inhibitor is administered orally, intravenously, intrathecally, or intraperitoneally.

Aspect 16: The pharmaceutical composition of any one of aspects 1-15, wherein the effective amount of an N-glycosylation inhibitor comprises about 10 mg to about 300 g of the N-glycosylation inhibitor.

Aspect 17: The pharmaceutical composition of any one of aspects 1-15, wherein the effective amount of an N-glycosylation inhibitor comprises about 200 mg to about 200 g of the N-glycosylation inhibitor.

Aspect 18: The pharmaceutical composition of any one of aspects 1-15, wherein the effective amount of an N-glycosylation inhibitor comprises about 5 g to about 100 g of the N-glycosylation inhibitor.

Aspect 19: The pharmaceutical composition of any one of aspects 1-18, wherein the pharmaceutical composition further comprises an effective amount of a pro-angiogenic factor.

Aspect 20: The pharmaceutical composition of aspect 19, wherein the pro-angiogenic factor comprises vascular endothelial growth factor (VEGF), or a derivative thereof.

Aspect 21: The pharmaceutical composition of aspect 20, wherein the VEGF is VEGF-A, VEGF-B, VEGF-C, VEGF-D, placental growth factor (PLGF), or a combination thereof.

Aspect 22: The pharmaceutical composition of aspect 20 or aspect 21, wherein the VEGF is a recombinant VEGF.

Aspect 23: The pharmaceutical composition any one of aspects 20-22, wherein the VEGF is administered locally near a site in the brain of a subject affected by an ischemic event.

Aspect 24: A method of treating stroke, the method comprising administering to a subject in need thereof an effective amount of an N-glycosylation inhibitor.

Aspect 25: The method of aspect 24, wherein the N-glycosylation inhibitor comprises hexosamine D-mannosamine (ManN), Kifunensine (Kif), Castanospermine (Cas), or a combination thereof.

Aspect 26: The method of aspect 25, wherein the N-glycosylation inhibitor comprises ManN.

Aspect 27: The method of any one of aspects 24-26, wherein the subject has experienced an ischemic stroke.

Aspect 28: The method of aspect 27, wherein the ischemic stroke is a thrombotic stroke or an embolic stroke.

Aspect 29: The method of any one of aspects 24-26, wherein the subject has experienced a hemorrhagic stroke.

Aspect 30: The method of aspect 29, wherein the hemorrhagic stroke comprises a subarachnoid hemorrhage or an intracerebral hemorrhage.

Aspect 31: The method of any one of aspects 24-26, wherein the subject has experienced a brainstem stroke.

Aspect 32: The method of any one of aspects 24-26, wherein the subject has experienced a transient ischemic attack.

Aspect 33: The method of any one of aspects 24-26, wherein the subject has experienced a cryptogenic stroke.

Aspect 34: The method of any one of aspects 24-33, wherein the effective amount of an N-glycosylation inhibitor comprises a single dosing treatment regimen.

Aspect 35: The method of aspect 34, wherein the single dosing treatment regimen comprises pretreatment, concurrent treatment, or post-treatment in relation to an ischemic event affecting the brain.

Aspect 36: The method of any one of aspects 24-35, wherein the effective amount of an N-glycosylation inhibitor comprises a multiple dosing treatment regimen.

Aspect 37: The method of aspect 36, wherein the multiple dosing treatment regimen comprises pretreatment, concurrent treatment, and/or post-treatment in relation to an ischemic event affecting the brain.

Aspect 38: The method of any one of aspects 24-37, wherein the effective amount of an N-glycosylation inhibitor is administered orally, intravenously, intrathecally or intraperitoneally.

Aspect 39: The method of any one of aspects 24-38, wherein the effective amount of an N-glycosylation inhibitor comprises about 10 mg to about 300 g of the N-glycosylation inhibitor.

Aspect 40: The method of any one of aspects 24-38, wherein the effective amount of an N-glycosylation inhibitor comprises about 200 mg to about 200 g of the N-glycosylation inhibitor.

Aspect 41: The method of any one of aspects 24-38, wherein the effective amount of an N-glycosylation inhibitor comprises about 5 g to about 100 g of the N-glycosylation inhibitor.

Aspect 42: The method of any one of aspects 34-41, wherein the single dosing treatment regimen comprises administering a formulation of the N-glycosylation inhibitor at a dosage of about 20 mg to about 900 mg/kg body weight of a subject.

Aspect 43: The method of any one of aspects 34-41, wherein the single dosing treatment regimen comprises administering a formulation of the N-glycosylation inhibitor at a dosage of about 200 mg to about 700 mg/kg body weight of a subject.

Aspect 44: The method of any one of aspects 36-41, wherein the multiple dosing treatment regimen comprises administering a formulation of the N-glycosylation inhibitor at a dosage of about 10 mg to about 900 mg/kg body weight of a subject.

Aspect 45: The method of any one of aspects 36-41, wherein the multiple dosing treatment regimen comprises administering a formulation of the N-glycosylation inhibitor at a dosage of about 200 mg to about 700 mg/kg body weight of a subject.

Aspect 46: The method of any one of aspects 24-45, wherein the administering to a subject further comprises administering an effective amount of a pro-angiogenic factor.

Aspect 47: The method of aspect 46, wherein the pro-angiogenic factor comprises vascular endothelial growth factor (VEGF), or a derivative thereof.

Aspect 48: The method of aspect 47, wherein the VEGF is VEGF-A, VEGF-B, VEGF-C, VEGF-D, placental growth factor (PLGF), or a combination thereof.

Aspect 49: The method of aspect 47 or aspect 48, wherein the VEGF is a recombinant VEGF.

Aspect 50: The method of any one of aspects 47-49, wherein the VEGF is administered locally near a site in the brain of a subject affected by an ischemic event.

Aspect 51: The method of any one of aspects 24-50, wherein N-linked glycosylation of proteins is inhibited in an endothelial cell in a subject.

Aspect 52: The method of any one of aspects 24-51, wherein the administration is effective to stimulate endothelial cell proliferation and angiogenesis of blood vessels near the brain.

Aspect 53: The method of any one of aspects 24-52, wherein the administration is effective to stimulate brain endothelial cell proliferation and angiogenesis.

Aspect 54: The method of any one of aspects 24-53, wherein the administration is effective to activate JNK signaling and upregulate an unfolded protein response caused by endoplasmic reticulum stress.

Aspect 55: A method of preventing stroke, the method comprising administering to a subject in need thereof an effective amount of an N-glycosylation inhibitor.

Aspect 56: The method of aspect 55, wherein the N-glycosylation inhibitor comprises hexosamine D-mannosamine (ManN), Kifunensine (Kif), Castanospermine (Cas), or a combination thereof.

Aspect 57: The method of aspect 56, wherein the N-glycosylation inhibitor comprises ManN.

Aspect 58: The method of any one of aspects 55-57, wherein the effective amount of an N-glycosylation inhibitor comprises a single dosing treatment regimen.

Aspect 59: The method of any one of aspects 55-57, wherein the effective amount of an N-glycosylation inhibitor comprises a multiple dosing treatment regimen.

Aspect 60: The method of any one of aspects 55-59, wherein the effective amount of an N-glycosylation inhibitor is administered orally, intravenously, intrathecally or intraperitoneally.

Aspect 61: The method of any one of aspects 55-60, wherein the effective amount of an N-glycosylation inhibitor comprises about 10 mg to about 300 g of the N-glycosylation inhibitor.

Aspect 62: The method of any one of aspects 55-60, wherein the effective amount of an N-glycosylation inhibitor comprises about 200 mg to about 200 g of the N-glycosylation inhibitor.

Aspect 63: The method of any one of aspects 55-60, wherein the effective amount of an N-glycosylation inhibitor comprises about 5 g to about 100 g of the N-glycosylation inhibitor.

Aspect 64: The method of any one of aspects 55-63, wherein a risk of recurrent stroke is reduced in a subject.

Aspect 65: The method of aspect 64, wherein a subject has previously experienced an ischemic stroke, a hemorrhagic stroke, a brainstem stroke, a transient ischemic attack, or a cryptogenic stroke.

Aspect 66: The method of aspect 64 or aspect 65, wherein the risk of a recurrent stroke comprises a risk of an ischemic stroke, a hemorrhagic stroke, a brainstem stroke, a transient ischemic attack, or a cryptogenic stroke.

Aspect 67: The method of any one of aspects 55-66, wherein a measurement of C-reactive protein (CRP) in the blood in a subject is reduced compared to a previous measurement of blood CRP level in the subject.

Aspect 68: The method of aspect 67, wherein the measurement of CRP in the blood in a subject is reduced to below about 10 mg/L.

Aspect 69: A method of attenuating an ongoing ischemic event affecting the brain, the method comprising administering to a subject in need thereof an effective amount of an N-glycosylation inhibitor.

Aspect 70: The method of aspect 69, wherein the N-glycosylation inhibitor comprises hexosamine D-mannosamine (ManN), Kifunensine (Kif), Castanospermine (Cas), or a combination thereof.

Aspect 71: The method of aspect 70, wherein the N-glycosylation inhibitor comprises ManN.

Aspect 72: The method of any one of aspects 69-71, wherein the subject is experiencing an ischemic stroke, a hemorrhagic stroke, a brainstem stroke, a transient ischemic attack, or a cryptogenic stroke.

Aspect 73: The method of any one of aspects 69-72, wherein the effective amount of an N-glycosylation inhibitor is administered orally, intravenously, intrathecally or intraperitoneally.

Aspect 74: The method of any one of aspects 69-73, wherein the effective amount of an N-glycosylation inhibitor comprises about 10 mg to about 300 g of the N-glycosylation inhibitor.

Aspect 75: The method of any one of aspects 69-73, wherein the effective amount of an N-glycosylation inhibitor comprises about 5 g to about 100 g of the N-glycosylation inhibitor.

Aspect 76: The method of any one of aspects 69-75, wherein a neurological assessment of acute stroke in a subject improves compared to a previous assessment.

Aspect 77: The method of any one of aspects 69-76, wherein blood flow to the brain of a subject is improved.

Aspect 78: The method of aspects 77, wherein the blood flow to the brain is assessed by transcranial doppler ultrasound.

Aspect 79: A method of treating stroke in a subject in need thereof comprising:
a) identifying a subject having suffered a stroke event;
b) administering to the subject a therapeutically effective amount of one or more N-glycosylation inhibitors;
c) determining whether the subject suffered an ischemic stroke or a hemorrhagic stroke;
d) administering a therapeutically effective amount of an anticoagulant if the subject was determined to have suffered an ischemic stroke or refraining from administering a therapeutically effective amount of an anticoagulant if the subject was determined to have suffered a hemorrhagic stroke; and
e) administering at least one N-glycosylation inhibitor for a duration of treatment; wherein the N-glycosylation inhibitors of step b) and step e) are the same or different.

Aspect 80: The method of aspect 79 wherein step c) comprises taking a CT scan or an MRI scan of the subject.

Aspect 81: The method of aspects 79 or 80 wherein in step e) the N-glycosylation inhibitor is administered from 1-7 times per week.

Aspect 82: The method of any one of aspects 79-81, wherein the duration of treatment in step e) ranges from about 1 day to about 12 weeks.

Aspect 83: The method of any one of aspects 79-82, wherein the size of infarction is reduced in the subject as compared with anticoagulant therapy alone, TPA therapy alone, or a combination of anticoagulant and TPA therapy alone.

Aspect 84: The method of any one of Aspects 79-83, wherein the disruption of the blood-brain barrier is reduced in the subject as compared with anticoagulant therapy alone, TPA therapy alone, or a combination of anticoagulant and TPA therapy alone.

Aspect 85: The method of any one of Aspects 79-84, wherein the hemorrhagic damage due to ischemic stroke is reduced in the subject as compared with anticoagulant therapy alone, TPA therapy alone, or a combination of anticoagulant and TPA therapy alone.

Aspect 86: The method of any one of Aspects 79-85, wherein mortality is reduced in the subject as compared with anticoagulant therapy alone, TPA therapy alone, or a combination of anticoagulant and TPA therapy alone.

Aspect 87: A method of treating degenerative (pathologic) myopia in a subject in need thereof, the method comprising administering to the subject an effective amount of one or more N-glycosylation inhibitors intravitreally, wherein one or more symptoms of degenerative (pathologic) myopia are improved following the administering.

Aspect 88: The method of aspect 88, wherein the one or more N-glycosylation inhibitors comprises ManN, Kif, Cas, or any combination thereof.

Aspect 89: The method of aspects 87 or 88, wherein the one or more N-glycosylation inhibitors comprises ManN.

Aspect 90: The method of any one of aspects 87-89, wherein the administering of the effective amount of one or more N-glycosylation inhibitors induces retinal angiogenesis in an eye of the subject.

Aspect 91: The method of any one of aspects 87-90, wherein the administering of the effective amount of one or more N-glycosylation inhibitors induces choroidal angiogenesis in an eye of the subject.

Aspect 92: The method of aspect 90 or 91, wherein the induced retinal angiogenesis or the induced choroidal angiogenesis restores vascular perfusion in an eye of the subject.

Aspect 93: The method of any one of aspects 87-92, wherein myopic choroidal neovascularization (CNV) is reduced in an eye of the subject.

Aspect 94: The method of any one of aspects 87-93, wherein a presence or a progression of chorioretinal atrophy (CRA) adjacent to an area of myopic CNV in the eye of the subject is reduced following the administering.

Aspect 95: The method of any one of aspects 87-94, wherein a progression of further visual impairment in the subject is reduced or halted following the administering.

Aspect 96: The method of any one of aspects 87-95, wherein the subject shows a slowing of disease progression of one of more symptoms selected from the group consisting of diffuse chorioretinal atrophy, patchy chorioretinal atrophy, lacquer cracks, myopic CNV, CNV-related macular atrophy, progression vision loss, foveal schisis, metamorphopsia, blurring, scotoma, difficulty reading, ocular floaters or flashes, loss of central vision, retinal thinning, difficulty recognizing faces, a gray spot in a visual field, and poor contrast sensitivity.

Aspect 97: The method of any one of aspects 87-96, wherein the subject shows a halting of disease progression of one of more symptoms selected from the group consisting of diffuse chorioretinal atrophy, patchy chorioretinal atrophy, lacquer cracks, myopic CNV, CNV-related macular atrophy, progression vision loss, foveal schisis, metamorphopsia, blurring, scotoma, difficulty reading, ocular floaters or flashes, loss of central vision, retinal thinning, difficulty recognizing faces, a gray spot in a visual field, and poor contrast sensitivity.

Aspect 98: The method of any one of aspects 87-97, wherein the subject shows an improvement in one of more symptoms selected from the group consisting of diffuse chorioretinal atrophy, patchy chorioretinal atrophy, lacquer cracks, myopic CNV, CNV-related macular atrophy, progression vision loss, foveal schisis, metamorphopsia, blurring, scotoma, difficulty reading, ocular floaters or flashes, loss of central vision, retinal thinning, difficulty recognizing faces, a gray spot in a visual field, and poor contrast sensitivity.

Aspect 99: The method of any one of aspects 87-98, wherein disease progression and effectiveness of treatment are monitored using spectral domain optical coherence tomography.

Aspect 100: The method of aspect 99, wherein the use of spectral domain optical coherence tomography in monitoring disease progression is used in part to determine a frequency of administration of the N-glycosylation inhibitor as part of a treatment regimen, a therapeutic dose of the N-glycosylation inhibitor to be administered to the subject, or a halting of the administering of the N-glycosylation inhibitor to the subject following an improvement in one or more symptoms of degenerative (pathologic) myopia.

1. A method of treating stroke, the method comprising administering to a subject in need thereof an effective amount of an N-glycosylation inhibitor.

2. The method of claim 1, wherein the N-glycosylation inhibitor comprises hexosamine D-mannosamine (ManN), Kifunensine (Kif), or Castanospermine (Cas), or a combination thereof.

3. The method of claim 1, wherein the N-glycosylation inhibitor comprises ManN.

4. The method of claim 1, wherein the subject has experienced an ischemic stroke.

5. The method of claim 4, wherein the ischemic stroke is a thrombotic stroke or an embolic stroke.

6. The method of claim 1, wherein the subject has experienced a hemorrhagic stroke.

7. The method of claim 6, wherein the hemorrhagic stroke comprises a subarachnoid hemorrhage or an intracerebral hemorrhage.

8. The method of claim 1, wherein the subject has experienced a brainstem stroke.

9. The method of claim 1, wherein the subject has experienced a transient ischemic attack.

10. The method of claim 1, wherein the subject has experienced a cryptogenic stroke.

11. The method of claim 1, wherein the effective amount of the N-glycosylation inhibitor is delivered to the subject via a single dosing treatment regimen or a multiple dosing treatment regimen.

12. The method of claim 11, wherein the single dosing treatment regimen or the multiple dosing treatment regimen comprises pretreatment, concurrent treatment, or post-treatment in relation to an ischemic event affecting a brain of the subject.

13-14. (canceled)

15. The method of claim 1, wherein the effective amount of the N-glycosylation inhibitor is administered orally, intravenously, intrathecally or intraperitoneally.

16. The method of claim 3, wherein an effective amount of ManN comprises about 10 mg to about 1 g per administration.

17-23. (canceled)

24. The method of claim 1, wherein the administering to the subject further comprises administering vascular endothelial growth factor (VEGF), or a derivative thereof.

25-27. (canceled)

28. The method of claim 1, wherein N-linked glycosylation of proteins is inhibited in an endothelial cell in the subject.

29. The method of claim 1, wherein the administering is effective to stimulate endothelial cell proliferation and angiogenesis of blood vessels near a brain or effective to stimulate brain endothelial cell proliferation and angiogenesis.

30-31. (canceled)

32. The method of claim 1, wherein the subject demonstrates an improvement in one or more symptoms of stroke following the administering, and wherein the one or more symptoms are selected from the group consisting of: muscle weakness, numbness in the face, paralysis in the face, paralysis in a limb, paralysis on one side of the body, slurred speech, garbled speech, difficulty understanding others, sudden onset blindness in one or both eyes, double vision, vertigo, confusion, lack of mental alertness, loss of balance, loss of coordination, stroke-induced mortality, hypoxic damage to one or more areas of the brain, stroke-induced memory impairment, stroke-induced voluntary movement impairment, stroke-induced language impairment, stroke-induced reduction in cognitive capacity, and stroke-induced reduction in mobility.

33. (canceled)

34. The method of claim 1, wherein the subject demonstrates a significant increase in vascular density in or near a region of stroke-induced brain injury following the administering.

35. A pharmaceutical composition for treating stroke, the pharmaceutical composition comprising an effective amount of an N-glycosylation inhibitor administered to a subject in need thereof.

36-81. (canceled)
